SEQUENCE LISTING (132 sequences)

SEQ ID NO: 1 - protein, 6 aa, synthetic construct
  SITE 1: note = D-isoleucine
  SITE 3..4: note = D-amino acid
IPFIPF

SEQ ID NO: 2 - protein, 6 aa, synthetic construct
  SITE 1..3: note = D-amino acid
  SITE 6: note = D-amino acid
PFAPPL

SEQ ID NO: 3 - protein, 6 aa, synthetic construct
  SITE 1..3: note = D-amino acid
  SITE 6: note = D-amino acid
  MOD_RES 2: note = N-methylated
PFAPPL

SEQ ID NO: 4 - protein, 6 aa, synthetic construct
  SITE 4: note = D-amino acid
  SITE 6: note = D-amino acid
PPVFPF

SEQ ID NO: 5 - protein, 6 aa, synthetic construct
  MOD_RES 3: note = N-methylated
  SITE 4: note = D-amino acid
  SITE 6: note = D-amino acid
PPVFPF

SEQ ID NO: 6 - protein, 6 aa, synthetic construct
  MOD_RES 1: note = N-methylated
  MOD_RES 3: note = N-methylated
  SITE 1..2: note = D-amino acid
  SITE 4: note = D-amino acid
FAPVFP

SEQ ID NO: 7 - protein, 6 aa, synthetic construct
  MOD_RES 3: note = N-methylated
  SITE 1..2: note = D-amino acid
  SITE 4: note = D-amino acid
VFLPFP

SEQ ID NO: 8 - protein, 6 aa, synthetic construct
  MOD_RES 1: note = N-methylated
  MOD_RES 3: note = N-methylated
  SITE 1..2: note = D-amino acid
  SITE 4: note = D-amino acid
VFLPFP

SEQ ID NO: 9 - protein, 6 aa, synthetic construct
  SITE 1: note = D-amino acid
  SITE 3..4: note = D-amino acid
  SITE 6: note = D-amino acid
  MOD_RES 3: note = N-methylated
  MOD_RES 6: note = N-methylated
IPFIPF

SEQ ID NO: 10 - protein, 6 aa, synthetic construct
  SITE 1..2: note = D-amino acid
  SITE 4: note = D-amino acid
FAPVFP

SEQ ID NO: 11 - protein, 7 aa, synthetic construct
  SITE 2..3: note = D-amino acid
  SITE 6: note = D-amino acid
  MOD_RES 2: note = N-methylated
PLVLPPI

SEQ ID NO: 12 - protein, 7 aa, synthetic construct
  SITE 1..2: note = D-amino acid
  SITE 6: note = D-amino acid
  MOD_RES 3: note = N-methylated
  MOD_RES 6: note = N-methylated
LPLIPLL

SEQ ID NO: 13 - protein, 7 aa, synthetic construct
  SITE 3..4: note = D-amino acid
  SITE 6..7: note = D-amino acid
  MOD_RES 3: note = N-methylated
LPLLPPL

SEQ ID NO: 14 - protein, 7 aa, synthetic construct
  SITE 1..2: note = D-amino acid
  SITE 5: note = D-amino acid
  MOD_RES 3: note = N-methylated
LPALPPL

SEQ ID NO: 15 - protein, 7 aa, synthetic construct
  SITE 3: note = D-amino acid
  SITE 6..7: note = D-amino acid
VIPFIPA

SEQ ID NO: 16 - protein, 7 aa, synthetic construct
  SITE 2..4: note = D-amino acid
  SITE 6..7: note = D-amino acid
  MOD_RES 5: note = N-methylated
PPLFLVF

SEQ ID NO: 17 - protein, 7 aa, synthetic construct
  SITE 3: note = D-amino acid
  SITE 6..7: note = D-amino acid
  MOD_RES 4: note = N-methylated
VIPFIPA

SEQ ID NO: 18 - protein, 8 aa, synthetic construct
  SITE 1: note = D-amino acid
  SITE 3..4: note = D-amino acid
  SITE 6: note = D-amino acid
LLPLLAPL

SEQ ID NO: 19 - protein, 8 aa, synthetic construct
  SITE 1..2: note = D-amino acid
  SITE 4..6: note = D-amino acid
  SITE 8: note = D-amino acid
  MOD_RES 4: note = N-methylated
  MOD_RES 8: note = N-methylated
LVPLLIPL

SEQ ID NO: 20 - protein, 8 aa, synthetic construct
  SITE 4..5: note = D-amino acid
  SITE 7..8: note = D-amino acid
FLPPLVPL

SEQ ID NO: 21 - protein, 8 aa, synthetic construct
  SITE 3: note = D-amino acid
  SITE 5..8: note = D-amino acid
  MOD_RES 6..7: note = N-methylated
LPPLVAAL

SEQ ID NO: 22 - protein, 8 aa, synthetic construct
  SITE 2..4: note = D-amino acid
  SITE 6..8: note = D-amino acid
  MOD_RES 2: note = N-methylated
  MOD_RES 6: note = N-methylated
PLLIPLIV

SEQ ID NO: 23 - protein, 8 aa, synthetic construct
  SITE 1..2: note = D-amino acid
  SITE 4: note = D-amino acid
  SITE 7..8: note = D-amino acid
  MOD_RES 3: note = N-methylated
  MOD_RES 6..7: note = N-methylated
PPAFALLL

SEQ ID NO: 24 - protein, 8 aa, synthetic construct
  SITE 1: note = D-amino acid
  SITE 3: note = D-amino acid
  SITE 5: note = D-amino acid
  SITE 7: note = D-amino acid
LVPPLVPP

SEQ ID NO: 25 - protein, 8 aa, synthetic construct
  SITE 2..3: note = D-amino acid
  SITE 6..8: note = D-amino acid
  MOD_RES 4: note = N-methylated
  MOD_RES 6: note = N-methylated
PLFFPLLP

SEQ ID NO: 26 - protein, 8 aa, synthetic construct
  SITE 2..3: note = D-amino acid
  SITE 6..7: note = D-amino acid
  MOD_RES 4: note = N-methylated
  MOD_RES 6: note = N-methylated
  MOD_RES 8: note = N-methylated
PLFFPLLG

SEQ ID NO: 27 - protein, 8 aa, synthetic construct
  SITE 1..2: note = D-amino acid
  SITE 4..5: note = D-amino acid
  SITE 7: note = D-amino acid
  MOD_RES 6: note = N-methylated
PLVPPLVI

SEQ ID NO: 28 - protein, 8 aa, synthetic construct
  SITE 2..3: note = D-amino acid
  SITE 5..7: note = D-amino acid
  MOD_RES 2: note = N-methylated
  MOD_RES 7: note = N-methylated
PLLFPPAI

SEQ ID NO: 29 - protein, 8 aa, synthetic construct
  SITE 1..2: note = D-amino acid
  SITE 4..5: note = D-amino acid
  SITE 7..8: note = D-amino acid
  MOD_RES 2..4: note = N-methylated
VLAAVPPI

SEQ ID NO: 30 - protein, 8 aa, synthetic construct
  SITE 2: note = D-amino acid
  SITE 5: note = D-amino acid
  SITE 7: note = D-amino acid
  MOD_RES 4..5: note = N-methylated
PPALLIVP

SEQ ID NO: 31 - protein, 8 aa, synthetic construct
  SITE 1..2: note = D-amino acid
  SITE 4..5: note = D-amino acid
  SITE 7..8: note = D-amino acid
  MOD_RES 3: note = N-methylated
  MOD_RES 7: note = N-methylated
PPLLPPLL

SEQ ID NO: 32 - protein, 8 aa, synthetic construct
  SITE 3..4: note = D-amino acid
  SITE 7..8: note = D-amino acid
PBLIPBLI

SEQ ID NO: 33 - protein, 8 aa, synthetic construct
  SITE 1..2: note = D-amino acid
  SITE 4: note = D-amino acid
  SITE 6..7: note = D-amino acid
  MOD_RES 3..4: note = N-methylated
  MOD_RES 7: note = N-methylated
IAALPVLP

SEQ ID NO: 34 - protein, 8 aa, synthetic construct
  SITE 1..2: note = D-amino acid
  SITE 4..8: note = D-amino acid
  MOD_RES 2..3: note = N-methylated
FALPLLPV

SEQ ID NO: 35 - protein, 8 aa, synthetic construct
  SITE 2..4: note = D-amino acid
  SITE 6..8: note = D-amino acid
  MOD_RES 3..5: note = N-methylated
PALFLVAP

SEQ ID NO: 36 - protein, 8 aa, synthetic construct
  SITE 1: note = D-amino acid
  SITE 3: note = D-amino acid
  SITE 5: note = D-amino acid
  SITE 7: note = D-amino acid
  MOD_RES 2: note = N-methylated
  MOD_RES 6: note = N-methylated
VLPLVLPL

SEQ ID NO: 37 - protein, 8 aa, synthetic construct
  SITE 3: note = D-amino acid
  SITE 7: note = D-amino acid
  MOD_RES 6..7: note = N-methylated
APPVALLV

SEQ ID NO: 38 - protein, 8 aa, synthetic construct
  SITE 1: note = D-amino acid
  SITE 3..5: note = D-amino acid
  SITE 7: note = D-amino acid
  MOD_RES 3..4: note = N-methylated
LVLLLVPP

SEQ ID NO: 39 - protein, 8 aa, synthetic construct
  SITE 1..7: note = D-amino acid
  MOD_RES 3: note = N-methylated
  MOD_RES 5: note = N-methylated
LPLALPLA

SEQ ID NO: 40 - protein, 8 aa, synthetic construct
  SITE 2..3: note = D-amino acid
  SITE 7: note = D-amino acid
  MOD_RES 4: note = N-methylated
FPPLIPPL

SEQ ID NO: 41 - protein, 8 aa, synthetic construct
  SITE 2: note = D-amino acid
  SITE 4..7: note = D-amino acid
  MOD_RES 6: note = N-methylated
MPPLLAVP

SEQ ID NO: 42 - protein, 8 aa, synthetic construct
  SITE 1..2: note = D-amino acid
  SITE 6..8: note = D-amino acid
  MOD_RES 3: note = N-methylated
  MOD_RES 6: note = N-methylated
  MOD_RES 8: note = N-methylated
APLLPLVA

SEQ ID NO: 43 - protein, 8 aa, synthetic construct
  SITE 1..8: note = D-amino acid
  MOD_RES 3: note = N-methylated
  MOD_RES 7: note = N-methylated
APLVAPLV

SEQ ID NO: 44 - protein, 8 aa, synthetic construct
  SITE 1..2: note = D-amino acid
  SITE 4..6: note = D-amino acid
  SITE 8: note = D-amino acid
  MOD_RES 8: note = N-methylated
FIPPIVPF

SEQ ID NO: 45 - protein, 9 aa, synthetic construct
  SITE 1: note = D-amino acid
  SITE 8: note = D-amino acid
  MOD_RES 8: note = N-methylated
PPBLBVPLI

SEQ ID NO: 46 - protein, 9 aa, synthetic construct
  SITE 2..3: note = D-amino acid
  SITE 5: note = D-amino acid
  SITE 7: note = D-amino acid
  SITE 9: note = D-amino acid
  MOD_RES 4: note = N-methylated
  MOD_RES 7: note = N-methylated
LIPLLPFVL

SEQ ID NO: 47 - protein, 8 aa, synthetic construct
  SITE 2: note = D-amino acid
  SITE 5..7: note = D-amino acid
  MOD_RES 5..7: note = N-methylated
FPPLLLLL

SEQ ID NO: 48 - protein, 9 aa, synthetic construct
  SITE 2..4: note = D-amino acid
  SITE 6..7: note = D-amino acid
  SITE 9: note = D-amino acid
  MOD_RES 3: note = N-methylated
  MOD_RES 8: note = N-methylated
PVAVPLVLF

SEQ ID NO: 49 - protein, 9 aa, synthetic construct
  SITE 2..3: note = D-amino acid
  SITE 9: note = D-amino acid
  MOD_RES 4..5: note = N-methylated
PAVLFIAPL

SEQ ID NO: 50 - protein, 9 aa, synthetic construct
  SITE 2: note = D-amino acid
  SITE 5..9: note = D-amino acid
  MOD_RES 1: note = N-methylated
  MOD_RES 4..5: note = N-methylated
LVLLFVPVP

SEQ ID NO: 51 - protein, 9 aa, synthetic construct
  SITE 2: note = D-amino acid
  SITE 6..7: note = D-amino acid
  SITE 9: note = D-amino acid
  MOD_RES 6..7: note = N-methylated
PLLLPAAIP

SEQ ID NO: 52 - protein, 9 aa, synthetic construct
  SITE 1: note = D-amino acid
  SITE 4..7: note = D-amino acid
  MOD_RES 3..4: note = N-methylated
  MOD_RES 6: note = N- Methylated
  MOD_RES 8: note = N-methylated
PAFLALVVP

SEQ ID NO: 53 - protein, 9 aa, synthetic construct
  SITE 2..3: note = D-amino acid
  SITE 5..8: note = D-amino acid
  MOD_RES 2..3: note = N-methylated
  MOD_RES 8: note = N-methylated
ALLPLPPLF

SEQ ID NO: 54 - protein, 9 aa, synthetic construct
  SITE 2: note = D-amino acid
  SITE 4..6: note = D-amino acid
  SITE 8: note = D-amino acid
  MOD_RES 2: note = N-methylated
  MOD_RES 4..5: note = N-methylated
  MOD_RES 8: note = N-methylated
PAVLLLLAP

SEQ ID NO: 55 - protein, 9 aa, synthetic construct
  SITE 1: note = D-amino acid
  SITE 4..8: note = D-amino acid
  MOD_RES 2..5: note = N-methylated
VAALLFPPP

SEQ ID NO: 56 - protein, 8 aa, synthetic construct
  SITE 1: note = D-amino acid
  SITE 3..5: note = D-amino acid
  MOD_RES 3: note = N-methylated
  SITE 7..8: note = D-amino acid
  MOD_RES 7: note = N-methylated
PPLLPPLL

SEQ ID NO: 57 - protein, 9 aa, synthetic construct
  SITE 2..3: note = D-amino acid
  SITE 5: note = D-amino acid
  SITE 7: note = D-amino acid
  SITE 9: note = D-amino acid
  MOD_RES 3..5: note = N-methylated
VFALLIPPA

SEQ ID NO: 58 - protein, 9 aa, synthetic construct
  SITE 1..2: note = D-amino acid
  SITE 7: note = D-amino acid
  MOD_RES 5: note = N-methylated
  MOD_RES 8: note = N-methylated
PIIFFPPLI

SEQ ID NO: 59 - protein, 9 aa, synthetic construct
  SITE 2: note = D-amino acid
  SITE 4..6: note = D-amino acid
  SITE 8: note = D-amino acid
  MOD_RES 4: note = N-methylated
PLIAVPPLP

SEQ ID NO: 60 - protein, 9 aa, synthetic construct
  SITE 2: note = D-amino acid
  SITE 4: note = D-amino acid
  SITE 6..9: note = D-amino acid
  MOD_RES 6..7: note = N-methylated
PPPAILLFA

SEQ ID NO: 61 - protein, 9 aa, synthetic construct
  SITE 2..3: note = D-amino acid
  SITE 5..8: note = D-amino acid
  MOD_RES 2: note = N-methylated
  MOD_RES 5: note = N-methylated
PAAVLLLPL

SEQ ID NO: 62 - protein, 9 aa, synthetic construct
  SITE 3: note = D-amino acid
  SITE 7..9: note = D-amino acid
  MOD_RES 8..9: note = N-methylated
APPVMPVLA

SEQ ID NO: 63 - protein, 9 aa, synthetic construct
  SITE 1..2: note = D-amino acid
  SITE 4..6: note = D-amino acid
  MOD_RES 2: note = N-methylated
  MOD_RES 4..5: note = n-methylated
PAVLAPPLP

SEQ ID NO: 64 - protein, 9 aa, synthetic construct
  SITE 1..3: note = D-amino acid
  SITE 6..7: note = D-amino acid
  SITE 9: note = D-amino acid
  MOD_RES 2: note = N-methylated
IAIPAVPPL

SEQ ID NO: 65 - protein, 9 aa, synthetic construct
  SITE 1..2: note = D-amino acid
  SITE 5..6: note = D-amino acid
  SITE 8..9: note = D-amino acid
  MOD_RES 5: note = N-methylated
  MOD_RES 8: note = N-methylated
PIPPLVILA

SEQ ID NO: 66 - protein, 9 aa, synthetic construct
  SITE 1: note = D-amino acid
  SITE 3: note = D-amino acid
  SITE 5: note = D-amino acid
  SITE 7: note = D-amino acid
  SITE 9: note = D-amino acid
  MOD_RES 4: note = N-methylated
  MOD_RES 7: note = N-methylated
LLPLLPAMA

SEQ ID NO: 67 - protein, 9 aa, synthetic construct
  SITE 1..2: note = D-amino acid
  SITE 4..7: note = D-amino acid
  SITE 9: note = D-amino acid
  MOD_RES 5..6: note = N-methylated
VPPALLAVP

SEQ ID NO: 68 - protein, 9 aa, synthetic construct
  SITE 1..2: note = D-amino acid
  SITE 5: note = D-amino acid
  SITE 7..8: note = D-amino acid
  MOD_RES 5: note = N-methylated
  MOD_RES 8: note = N-methylated
PLAPALILV

SEQ ID NO: 69 - protein, 9 aa, synthetic construct
  SITE 1: note = D-amino acid
  SITE 3..4: note = D-amino acid
  MOD_RES 4..5: note = N-methylated
  MOD_RES 1: note = N-methylated
LVLLFVPGP

SEQ ID NO: 70 - protein, 9 aa, synthetic construct
  SITE 1: note = D-amino acid
  SITE 3..5: note = D-amino acid
  SITE 8: note = D-amino acid
  MOD_RES 6: note = N-methylated
PALLPFLVP

SEQ ID NO: 71 - protein, 9 aa, synthetic construct
  SITE 1: note = D-amino acid
  SITE 5: note = D-amino acid
  SITE 8: note = D-amino acid
  MOD_RES 8: note = N-methylated
PPGILVPAI

SEQ ID NO: 72 - protein, 9 aa, synthetic construct
  SITE 1: note = D-amino acid
  SITE 3: note = D-amino acid
  SITE 5: note = D-amino acid
  SITE 8: note = D-amino acid
  MOD_RES 5: note = N-methylated
  MOD_RES 8: note = N-methylated
PMLPLLFLV

SEQ ID NO: 73 - protein, 9 aa, synthetic construct
  SITE 2: note = D-amino acid
  SITE 5..6: note = D-amino acid
  SITE 8: note = D-amino acid
  MOD_RES 5: note = N-methylated
PPAPLAFIF

SEQ ID NO: 74 - protein, 9 aa, synthetic construct
  SITE 1: note = D-amino acid
  SITE 3..5: note = D-amino acid
  SITE 7: note = D-amino acid
  MOD_RES 4: note = N-methylated
  MOD_RES 6: note = N-methylated
PILLPFVVP

SEQ ID NO: 75 - protein, 10 aa, synthetic construct
  SITE 2: note = D-amino acid
  SITE 4..5: note = D-amino acid
  SITE 7..8: note = D-amino acid
  SITE 10: note = D-amino acid
  MOD_RES 3: note = N-methylated
  MOD_RES 6: note = N-methylated
  MOD_RES 8: note = N-methylated
IPFAPLVLPV

SEQ ID NO: 76 - protein, 10 aa, synthetic construct
  SITE 2: note = D-amino acid
  SITE 4: note = D-amino acid
  SITE 7..8: note = D-amino acid
  MOD_RES 5: note = N-methylated
  MOD_RES 7: note = N-methylated
  MOD_RES 9: note = N-methylated
PPAVLALPVV

SEQ ID NO: 77 - protein, 10 aa, synthetic construct
  SITE 1: note = D-amino acid
  SITE 9: note = D-amino acid
  MOD_RES 3: note = N-methylated
  MOD_RES 6: note = N-methylated
  MOD_RES 8: note = N-methylated
LLLLPLAALP

SEQ ID NO: 78 - protein, 10 aa, synthetic construct
  SITE 4: note = D-amino acid
  SITE 6: note = D-amino acid
  SITE 8: note = D-amino acid
  SITE 10: note = D-amino acid
  MOD_RES 1..2: note = N-methylated
  MOD_RES 5: note = N-methylated
LFLPLIPPAV

SEQ ID NO: 79 - protein, 10 aa, synthetic construct
  SITE 1: note = D-amino acid
  SITE 4: note = D-amino acid
  SITE 8: note = D-amino acid
  MOD_RES 5: note = N-methylated
  MOD_RES 8: note = N-methylated
  MOD_RES 10: note = N-methylated
ALVPLVPLVI

SEQ ID NO: 80 - protein, 10 aa, synthetic construct
  SITE 2: note = D-amino acid
  SITE 5..6: note = D-amino acid
  MOD_RES 2: note = N-methylated
  MOD_RES 5: note = N-methylated
  MOD_RES 9..10: note = N-methylated
PLAPLFLVLL

SEQ ID NO: 81 - protein, 10 aa, synthetic construct
  SITE 1: note = D-amino acid
  SITE 3: note = D-amino acid
  SITE 7: note = D-amino acid
  SITE 9: note = D-amino acid
  MOD_RES 3..5: note = N-methylated
FALALPPVIP

SEQ ID NO: 82 - protein, 10 aa, synthetic construct
  SITE 2: note = D-amino acid
  SITE 7: note = D-amino acid
  SITE 10: note = D-amino acid
  MOD_RES 3..4: note = N-methylated
  MOD_RES 9: note = N-methylated
PIALLPPALF

SEQ ID NO: 83 - protein, 10 aa, synthetic construct
  SITE 1: note = D-amino acid
  SITE 3..5: note = D-amino acid
  SITE 7: note = D-amino acid
  SITE 9: note = D-amino acid
  MOD_RES 2: note = N-methylated
  MOD_RES 8..9: note = N-methylated
LAPPPPVLLA

SEQ ID NO: 84 - protein, 10 aa, synthetic construct
  SITE 2: note = D-amino acid
  SITE 4: note = D-amino acid
  SITE 6..7: note = D-amino acid
  SITE 9..10: note = D-amino acid
  MOD_RES 6..8: note = N-methylated
FPPVVLAVPA

SEQ ID NO: 85 - protein, 10 aa, synthetic construct
  SITE 2: note = D-amino acid
  SITE 4: note = D-amino acid
  SITE 7..10: note = D-amino acid
  MOD_RES 2: note = N-methylated
  MOD_RES 9: note = N-methylated
PLFIPLLPAV

SEQ ID NO: 86 - protein, 10 aa, synthetic construct
  SITE 2..3: note = D-amino acid
  SITE 6: note = D-amino acid
  SITE 9..10: note = D-amino acid
  MOD_RES 2: note = N-methylated
  MOD_RES 9: note = N-methylated
FALIPPVPAL

SEQ ID NO: 87 - protein, 10 aa, synthetic construct
  SITE 2..3: note = D-amino acid
  SITE 5..6: note = D-amino acid
  SITE 8: note = D-amino acid
  MOD_RES 2: note = N-methylated
  MOD_RES 4: note = N-methylated
  MOD_RES 9: note = N-methylated
PAFLLPPALV

SEQ ID NO: 88 - protein, 10 aa, synthetic construct
  SITE 1..3: note = D-amino acid
  SITE 5: note = D-amino acid
  SITE 8: note = D-amino acid
  SITE 10: note = D-amino acid
  MOD_RES 4..5: note = N-methylated
FIPALLLPPL

SEQ ID NO: 89 - protein, 10 aa, synthetic construct
  SITE 1..2: note = D-amino acid
  SITE 4..7: note = D-amino acid
  SITE 9: note = D-amino acid
  MOD_RES 2: note = N-methylated
LALFIPAPPL

SEQ ID NO: 90 - protein, 10 aa, synthetic construct
  SITE 2: note = D-amino acid
  SITE 4..8: note = D-amino acid
  MOD_RES 2..4: note = N-methylated
MALALPAPPL

SEQ ID NO: 91 - protein, 10 aa, synthetic construct
  SITE 1..5: note = D-amino acid
  SITE 7: note = D-amino acid
  SITE 9: note = D-amino acid
  MOD_RES 8: note = N-methylated
  MOD_RES 10: note = N-methylated
VLPVPPPLFL

SEQ ID NO: 92 - protein, 10 aa, synthetic construct
  SITE 4: note = D-amino acid
  SITE 6: note = D-amino acid
  SITE 10: note = D-amino acid
  MOD_RES 1..2: note = N-methylated
  MOD_RES 5: note = N-methylated
  SITE 8: note = D-amino acid
GFLPLIPPAV

SEQ ID NO: 93 - protein, 10 aa, synthetic construct
  SITE 1..3: note = D-amino acid
  SITE 5..6: note = D-amino acid
  SITE 8: note = D-amino acid
  MOD_RES 5..6: note = N-methylated
VAFILAPPPM

SEQ ID NO: 94 - protein, 10 aa, synthetic construct
  SITE 2: note = D-amino acid
  SITE 6: note = D-amino acid
  SITE 8: note = D-amino acid
  MOD_RES 1..2: note = N-methylated
  MOD_RES 5: note = N-methylated
  SITE 4: note = D-amino acid
  SITE 10: note = D-amino acid
LFLPLIPPAV

SEQ ID NO: 95 - protein, 10 aa, synthetic construct
  SITE 3..4: note = D-amino acid
  SITE 6: note = D-amino acid
  MOD_RES 3: note = N-methylated
  SITE 1: note = D-amino acid
APFPFPPALL

SEQ ID NO: 96 - protein, 10 aa, synthetic construct
  MOD_RES 5..6: note = N-methylated
  SITE 1: note = D-amino acid
  SITE 3..4: note = D-amino acid
  SITE 7..9: note = D-amino acid
AGPFFFFAFP

SEQ ID NO: 97 - protein, 10 aa, synthetic construct
  SITE 2..3: note = D-amino acid
  SITE 7..8: note = D-amino acid
PVPVVPVPFF

SEQ ID NO: 98 - protein, 10 aa, synthetic construct
  SITE 2..3: note = D-amino acid
  SITE 5: note = D-amino acid
  SITE 7: note = D-amino acid
  SITE 9: note = D-amino acid
  MOD_RES 4: note = N-methylated
  MOD_RES 7: note = N-methylated
  MOD_RES 9: note = N-methylated
LLPAPPLALV

SEQ ID NO: 99 - protein, 10 aa, synthetic construct
  SITE 2..3: note = D-amino acid
  SITE 5: note = D-amino acid
  SITE 7..10: note = D-amino acid
  MOD_RES 2..3: note = N-methylated
  MOD_RES 9: note = N-methylated
PALALPPILI

SEQ ID NO: 100 - protein, 10 aa, synthetic construct
  SITE 1..3: note = D-amino acid
  SITE 5: note = D-amino acid
  SITE 7: note = D-amino acid
  SITE 9..10: note = D-amino acid
  MOD_RES 2: note = N-methylated
  MOD_RES 6: note = N-methylated
  MOD_RES 9: note = N-methylated
PLVIPAPLLL

SEQ ID NO: 101 - protein, 10 aa, synthetic construct
  SITE 1..2: note = D-amino acid
  SITE 4..7: note = D-amino acid
  SITE 9: note = D-amino acid
  MOD_RES 6..7: note = N-methylated
APPVLLLALP

SEQ ID NO: 102 - protein, 10 aa, synthetic construct
  SITE 2..3: note = D-amino acid
  SITE 5: note = D-amino acid
  SITE 7..10: note = D-amino acid
  MOD_RES 5..6: note = N-methylated
PVIPALPLLI

SEQ ID NO: 103 - protein, 10 aa, synthetic construct
  SITE 1: note = D-amino acid
  SITE 5..6: note = D-amino acid
  SITE 9: note = D-amino acid
  MOD_RES 5: note = N-methylated
  MOD_RES 9: note = N-methylated
APVPFAAVFL

SEQ ID NO: 104 - protein, 10 aa, synthetic construct
  SITE 3..5: note = D-amino acid
  SITE 7: note = D-amino acid
  SITE 10: note = D-amino acid
  MOD_RES 4: note = N-methylated
  MOD_RES 6: note = N-methylated
LVFLPFIPLP

SEQ ID NO: 105 - protein, 10 aa, synthetic construct
  SITE 4..5: note = D-amino acid
  SITE 8: note = D-amino acid
  MOD_RES 1..2: note = N-methylated
  MOD_RES 4: note = N-methylated
  MOD_RES 6: note = N-methylated
  MOD_RES 8: note = N-methylated
LGPLALPLLF

SEQ ID NO: 106 - protein, 10 aa, synthetic construct
  SITE 1: note = D-amino acid
  SITE 6: note = D-amino acid
  SITE 8: note = D-amino acid
  MOD_RES 1..2: note = N-methylated
  MOD_RES 6..7: note = N-methylated
  MOD_RES 9..10: note = N-methylated
FLPAVLFIAA

SEQ ID NO: 107 - protein, 10 aa, synthetic construct
  SITE 4: note = D-amino acid
  SITE 1: note = D-amino acid
  MOD_RES 8: note = N-methylated
  MOD_RES 5: note = N-methylated
  MOD_RES 10: note = N-methylated
LILPFVPGFL

SEQ ID NO: 108 - protein, 10 aa, synthetic construct
  SITE 4..5: note = D-amino acid
  SITE 9: note = D-amino acid
  MOD_RES 6: note = N-methylated
  MOD_RES 8: note = N-methylated
  SITE 2: note = D-amino acid
LFPLALLAFP

SEQ ID NO: 109 - protein, 11 aa, synthetic construct
  SITE 3: note = D-amino acid
  SITE 5..7: note = D-amino acid
  SITE 10..11: note = D-amino acid
  MOD_RES 5: note = N-methylated
  MOD_RES 8: note = N-methylated
VPPLIIPLIIP

SEQ ID NO: 110 - protein, 11 aa, synthetic construct
  SITE 3: note = D-amino acid
  SITE 5..7: note = D-amino acid
  SITE 10..11: note = D-amino acid
  MOD_RES 5: note = N-methylated
  MOD_RES 8: note = N-methylated
VPPLIIPFIIP

SEQ ID NO: 111 - protein, 11 aa, synthetic construct
  SITE 3: note = D-amino acid
  SITE 5..6: note = D-amino acid
  SITE 8: note = D-amino acid
  SITE 10: note = D-amino acid
  MOD_RES 3: note = N-methylated
  MOD_RES 8..9: note = N-methylated
  MOD_RES 11: note = N-methylated
APLVLIPLAPL

SEQ ID NO: 112 - protein, 11 aa, synthetic construct
  SITE 3: note = D-amino acid
  MOD_RES 2..3: note = N-methylated
  MOD_RES 9..10: note = N-methylated
  SITE 5..6: note = D-amino acid
  SITE 8..10: note = D-amino acid
VLLAIPPFALP

SEQ ID NO: 113 - protein, 11 aa, synthetic construct
  SITE 2: note = D-amino acid
  SITE 4..7: note = D-amino acid
  SITE 10..11: note = D-amino acid
  MOD_RES 2..3: note = N-methylated
VLLPLAPPAFL

SEQ ID NO: 114 - protein, 11 aa, synthetic construct
  SITE 1: note = D-amino acid
  SITE 4..5: note = D-amino acid
  SITE 7: note = D-amino acid
  SITE 10: note = D-amino acid
  MOD_RES 3: note = N-methylated
  MOD_RES 9: note = N-methylated
  MOD_RES 11: note = N-methylated
PIAIPPLIAVL

SEQ ID NO: 115 - protein, 11 aa, synthetic construct
  SITE 2..5: note = D-amino acid
  SITE 7: note = D-amino acid
  SITE 11: note = D-amino acid
  MOD_RES 4: note = N-methylated
  MOD_RES 6..7: note = N-methylated
PPPLFLLIAPV

SEQ ID NO: 116 - protein, 11 aa, synthetic construct
  SITE 2: note = D-amino acid
  SITE 4..7: note = D-amino acid
  MOD_RES 5: note = N-methylated
  MOD_RES 10: note = N-methylated
PPVVLLPPIAI

SEQ ID NO: 117 - protein, 12 aa, synthetic construct
  SITE 3..4: note = D-amino acid
  SITE 9..10: note = D-amino acid
  MOD_RES 9: note = N-methylated
  MOD_RES 3: note = N-methylated
  MOD_RES 12: note = N-methylated
  SITE 12: note = D-amino acid
VPLIPVAPVLPA

SEQ ID NO: 118 - protein, 11 aa, synthetic construct
  SITE 2..3: note = D-amino acid
  SITE 6..11: note = D-amino acid
  MOD_RES 4: note = N-methylated
  MOD_RES 6: note = N-methylated
  MOD_RES 9: note = N-methylated
  MOD_RES 11: note = N-methylated
LPPLLALPLAL

SEQ ID NO: 119 - protein, 11 aa, synthetic construct
  MOD_RES 3..4: note = N-methylated
  MOD_RES 7: note = N-methylated
  SITE 2: note = D-amino acid
  SITE 4..8: note = D-amino acid
  SITE 10..11: note = D-amino acid
LFLAPPLVPPA

SEQ ID NO: 120 - protein, 11 aa, synthetic construct
  SITE 2: note = D-amino acid
  SITE 5..7: note = D-amino acid
  MOD_RES 9: note = D-amino acid & N-methylated
  MOD_RES 2: note = N-methylated
  MOD_RES 6..7: note = N-methylated
PALPPALFLVI

SEQ ID NO: 121 - protein, 11 aa, synthetic construct
  SITE 4..5: note = D-amino acid
  SITE 7: note = D-amino acid
  SITE 9..11: note = D-amino acid
  MOD_RES 3..4: note = N-methylated
  MOD_RES 9: note = N-methylated
PIVAPPLVLFP

SEQ ID NO: 122 - protein, 11 aa, synthetic construct
  SITE 1..4: note = D-amino acid
  SITE 8..9: note = D-amino acid
  MOD_RES 3: note = N-methylated
  MOD_RES 5: note = N-methylated
  MOD_RES 8: note = N-methylated
VPVLIIPLIIP

SEQ ID NO: 123 - protein, 11 aa, synthetic construct
  MOD_RES 6: note = N-methylated
  MOD_RES 9: note = N-methylated
  SITE 1..2: note = D-amino acid
  SITE 4..5: note = D-amino acid
  SITE 7: note = D-amino acid
  SITE 9..10: note = D-amino acid
IPLLPLFLAIP

SEQ ID NO: 124 - protein, 11 aa, synthetic construct
  SITE 1..5: note = D-amino acid
  SITE 7..11: note = D-amino acid
  MOD_RES 3: note = N-methylated
IPLAPPPLALL

SEQ ID NO: 125 - protein, 11 aa, synthetic construct
  SITE 1..5: note = D-amino acid
  SITE 7..11: note = D-amino acid
  MOD_RES 3: note = N-methylated
LPFAPPPAALL

SEQ ID NO: 126 - protein, 11 aa, synthetic construct
  SITE 1..2: note = D-amino acid
  SITE 4..5: note = D-amino acid
  SITE 7: note = D-amino acid
  SITE 9..10: note = D-amino acid
  MOD_RES 6: note = N-methylated
  MOD_RES 9: note = N-methylated
IPLLPLFLGIP

SEQ ID NO: 127 - protein, 12 aa, synthetic construct
  SITE 1: note = D-amino acid
  SITE 4: note = D-amino acid
  SITE 6: note = D-amino acid
  SITE 8: note = D-amino acid
  SITE 10..11: note = D-amino acid
  MOD_RES 2: note = N-methylated
  MOD_RES 11: note = N-methylated
PFIPLLLPPAAI

SEQ ID NO: 128 - protein, 12 aa, synthetic construct
  SITE 6: note = D-amino acid
  SITE 4: note = D-amino acid
  SITE 1: note = D-amino acid
  SITE 8: note = D-amino acid
  SITE 10..11: note = D-amino acid
  MOD_RES 2: note = N-methylated
  MOD_RES 11: note = N-methylated
PVIPLFLPPALI

SEQ ID NO: 129 - protein, 8 aa, synthetic construct
  SITE 1: note = D-amino acid
  SITE 3..5: note = D-amino acid
  SITE 7..8: note = D-amino acid
  MOD_RES 3: note = N-methylated
  MOD_RES 7: note = N-methylated
PPLLPPLL

SEQ ID NO: 130 - protein, 9 aa, synthetic construct
  SITE 1..2: note = D-amino acid
  SITE 4..6: note = D-amino acid
  MOD_RES 3..4: note = N-methylated
PVMALPPLP

SEQ ID NO: 131 - protein, 12 aa, synthetic construct
  SITE 3..4: note = D-amino acid
  SITE 9..10: note = D-amino acid
  SITE 12: note = D-amino acid
  MOD_RES 3: note = N-methylated
  MOD_RES 12: note = N-methylated
  MOD_RES 9: note = N-methylated
VPLIPVAPVLPA

SEQ ID NO: 132 - protein, 6 aa, synthetic construct
  SITE 2: note = D-amino acid
  SITE 5: note = D-amino acid
IPFIPF

(19) United States
(12) Patent Application Publication
(10) Pub. No.: US 2026/0274895 A1
(43) Pub. Date: Sep. 17, 2026

(54) DE NOVO DESIGNED MEMBRANE-TRAVERSING AND ORALLY BIOAVAILABLE MACROCYCLES

(71) Applicants: UNIVERSITY OF WASHINGTON, Seattle, WA (US); The Simons Foundation, Inc., New York, NY (US)

(72) Inventors: David BAKER, Seattle, WA (US); Gaurav BHARDWAJ, Seattle, WA (US); Jacob O'CONNOR, Seattle, WA (US); Sephen RETTIE, Seattle, WA (US); Lance Joseph STEWART, Seattle, WA (US); Gizem GOKCE, Seattle, WA (US); Jonathan PALMER, Seattle, WA (US); Vikram MULLIGAN, New York, NY (US)

(21) Appl. No.: 18/727,621

(22) PCT Filed: Jan. 11, 2023
(86) PCT No.: PCT/US2023/060479

§ 371 Date: Jul. 9, 2024

Related U.S. Application Data
(60) Provisional application No. 63/299,219, filed on Jan. 13, 2022.

(51) Int. Cl. C07K 7/64; A61K 47/64; C40B 40/10

(52) U.S. Cl. CPC C07K 7/64; A61K 47/64; C40B 40/10

(57) ABSTRACT

Cyclic peptides are disclosed that comprise an amino acid sequence having no more than 3 amino acid substitutions compared to the amino acid sequence selected from the group consisting of SEQ ID NO: 1-123 and 125-131 or mirror images thereof.

### CROSS REFERENCE

This application claims priority to U.S. Provisional Patent Application Ser. No. 63/299,219 filed Jan. 13, 2022, incorporated by reference herein in its entirety.

### SEQUENCE LISTING STATEMENT

A computer readable form of the Sequence Listing is filed with this application by electronic submission and is incorporated into this application by reference in its entirety. The Sequence Listing is contained in the file created on Jan. 6, 2023 having the file name “21-1372-WO.xml” and is 323 kb in size.

### BACKGROUND

Cyclic peptides have considerable potential advantages as therapeutics since, unlike small molecules, they can disrupt protein-protein interactions, and, unlike proteins, they can traverse biological membranes. However, realizing these advantages is complicated by the difficulty to date in robustly designing peptides with diverse shapes and sizes to be membrane permeable.

### SUMMARY

In one aspect, the disclosure provides cyclic peptides comprising or consisting of an amino acid sequence having no more than 3 amino acid substitutions compared to the amino acid sequence selected from the group consisting of SEQ ID NO: 1-123 and 125-131 or mirror images thereof, wherein residues in uppercase are L amino acids, and residues in lower case are D amino acids, wherein an asterisk identifies the amino acid residue following the asterisk as being an N-methylated amino acid. In one embodiment, the cyclic peptide is cell membrane permeable. In another embodiment, the cyclic peptide comprises or consists of an amino acid sequence having no more than 3 amino acid substitutions compared to the amino acid sequence selected from the group consisting SEQ ID NO:19, 36, 43, 61, 109, or mirror images thereof. In a further embodiment, the cyclic peptide has at least one proline residue and/or at least one N-methylated amino acid. In one embodiment, the cyclic peptide has no N-methylated amino acids. In another embodiment, the peptide comprises at least 1, 2, 3, 4, or more D amino acids. In a further embodiment, the cyclic peptide is 6-12 amino acid residues in length.

In another aspect, the disclosure provides cyclic peptides comprising or consisting of a bin string as listed in Table 3, wherein, wherein A is a right-handed helical region; B is a right-handed strand region; X is a mirror image of A; Y is a mirror image of B; O are amino acids with phi <0 and cis peptide bond between the residue i and i+1; and Z are amino acids with phi >0 and cis peptide bond between the residue i and i+1.

In one embodiment of all embodiments herein, the cyclic peptide has an apparent permeability greater than 1×10⁻⁷ cm/s. In a further embodiment, the apparent permeability is measured by a rate of traversal across artificial membranes in parallel artificial membrane permeability assays (PAMPA), determined by mass spectrometry-based quantification of peptide concentrations in the donor and acceptor wells. In another embodiment, the disclosure provides conjugates comprising the cyclic peptide of any embodiment herein bound to a moiety. In various embodiments, the moiety comprises a therapeutic agent, a diagnostic agent, a marker, a linker, a dye, a purification tag, a peptide, a small molecule, or a nucleic acid.

In a further embodiment, the disclosure provides cyclic peptide libraries, comprising two or more cyclic peptides and/or conjugates according to any embodiment disclosed herein. In another embodiment, the disclosure provides methods for use of the cyclic peptide, conjugate, or cyclic peptide library of any embodiment herein to carry a linked moiety across a cell membrane, or as a scaffold for target-based drug design, or to screen molecules of interest for binding to one or more of the cyclic peptides.

### DESCRIPTION OF THE FIGURES

FIG. 1. Computational design and structure validation of 6-8 amino acid macrocycles Structural validation of computationally designed macrocycles. Each panel shows the design model and torsion bin string describing the design model (left), hydrogen bonding pattern for the design model (middle), and superposition between the design model and the X-ray structure (right). For hydrogen bonding graphs, the boxes highlight the designed intramolecular hydrogen bonds. Amino acids without a backbone hydrogen bond donor (proline, D-proline, N-methylated amino acids) are marked by darker columns. Sidechains for non-proline residues are not shown in superposition graphs. RMSD between the design model and X-ray structure was calculated over all backbone heavy atoms (C, CA, N, O, CN). *in the macrocycle sequence denotes the N-methylated amino acid positions and lower-case denotes the D-amino acids.

FIG. 2. Permeability measurements of computationally designed macrocycles in PAMPA assays. (A) Apparent permeability (Pₐₚₚ) of 6-12 amino acid macrocycles in PAMPA assay. Peptides are grouped based on sequence length. Isobaric peaks (denoted p1 and p2) were seen for some peptides during purification and were assayed separately. Bar height: Average Pₐₚₚ from three replicates, Error bars: standard deviation calculated from three replicates.

FIG. 3. Computational design and structure validation of 9-12 amino acid macrocycles. Structural validation of computationally-designed macrocycles. Each panel shows the design model and torsion bin string describing the design model (left), hydrogen bonding pattern for the design model (middle), and superposition between the design model and the X-ray structure (right). For hydrogen bonding graphs, the boxes highlight the designed intramolecular hydrogen bonds. Amino acids without a backbone hydrogen bond donor (Proline, D-proline, N-methylated amino acids) are marked by darker columns. Sidechains for non-proline residues are not shown in superposition graphs. RMSD between the design model and X-ray structure was calculated over all the backbone heavy atoms (C, CA, N, O, CN). *in macrocycle sequence denotes the N-methylated amino acid positions and lower-case denotes the D-amino acids.

FIG. 4. Design and structural characterization of conformation switching macrocycles. Design models and experimentally determined structures (X-ray) for different conformational states of designs D8.31 (left), D8.21 (middle), and D9.16 (right). The design model and predicted low-energy states are shown (labelled LE_*). Superposition between the predicted low-energy states and the experimental structures is shown in the boxes.

FIG. 5 Designed macrocycles are orally bioavailable in vivo in rodent models Plasma concentration of unmodified full-length peptides measured after intravenous (IV), subcutaneous (SQ), and oral (PO) administration in mice (D8.3.p1, D10.1, and D11.3) (n=3 mice for each dosing route).

FIG. 6. Overall schematic of the in silico pipeline for design of membrane-permeable peptides: Design process starts with a linear polyglycine peptide chain that is cyclized using Rosetta™ generalized kinematic closure (genKIC) protocol. Iterative rounds of amino acid sequence design and N-methylation of non hydrogen-bonded NH groups are performed to design low-energy macrocycles with no unsatisfied backbone NH groups. The process is repeated to sample 10⁵-10⁶ design models that are clustered to identify permeable macrocycles with diverse shapes and sizes.

FIG. 7. An example Energy vs RMSD to Design plot from structure prediction runs using Rosetta™ simple_cycpep_predict application: Diverse conformations for a given amino acid sequence are generated using generalized kinematic closure (genKIC) protocol and energy-minimized using Rosetta™ FastRelax protocol. Each point represents an independently predicted structure. Dots represent the local minimization of the designed macrocycle structure. Landscapes that funnel into the design structure as the lowest energy structure and have a big energy gap (delta E) between the designed fold and other unfolded states are selected for experimental characterization.

FIG. 8. Structure-energy landscapes and hydrogen-bonding patterns for designed and selected 6-amino acid macrocycles: Left panels show the torsion bin cluster and predicted conformational landscape for each design. * denotes N-methylated amino acids. Points denote an independently predicted structure with no cis-peptide bonds. Points represent predicted structures with at least one cis-peptide bond. Dots represent the local minimization of the designed macrocycle structure. For hydrogen bonding graphs, the boxes highlight the designed intramolecular hydrogen bonds. Amino acids without a backbone hydrogen bond donor (proline, D-proline, N-methylated amino acids) are marked by darker columns.

FIG. 9. Structure-energy landscapes and hydrogen-bonding patterns for designed and selected 7-amino acid macrocycles: Left panels show the torsion bin cluster and predicted conformational landscape for each design. * denotes N-methylated amino acids. Points denote an independently predicted structure with no-peptide bonds. Points represent predicted structures with at least one cis-peptide bond. Blue dots represent the local minimization of the designed macrocycle structure. For hydrogen bonding graphs, the boxes highlight the designed intramolecular hydrogen bonds. Amino acids without a backbone hydrogen bond donor (proline, D-proline, N-methylated amino acids) are marked by darker columns.

FIG. 10. Structure-energy landscapes and hydrogen-bonding patterns for designed and selected 8-amino acid macrocycles: Left panels show the torsion bin cluster and predicted conformational landscape for each design. * denotes N-methylated amino acids. Points denote an independently predicted structure with no cis-peptide bonds. Points represent predicted structures with at least one cis-peptide bond. Dots represent the local minimization of the designed macrocycle structure. For hydrogen bonding graphs, the boxes highlight the designed intramolecular hydrogen bonds. Amino acids without a backbone hydrogen bond donor (proline, D-proline, N-methylated amino acids) are marked by darker columns.

FIG. 11. Examples of geometrically strained arrangement of overlapping gamma and beta turns seen in some of the design models. For such designs, variants with N-methylated middle residue were also generated and tested experimentally. N-methyls are shown. Intramolecular hydrogen bonding interactions shown as dashes.

FIG. 12. Structure-energy landscapes and hydrogen-bonding patterns for designed and selected 9-amino acid macrocycles. Left panels show the torsion bin cluster and predicted conformational landscape for each design. * denotes N-methylated amino acids. Points denote an independently predicted structure with no-peptide bonds. Points represent predicted structures with at least one cis-peptide bond. Blue dots represent the local minimization of the designed macrocycle structure. For hydrogen bonding graphs, the boxes highlight the designed intramolecular hydrogen bonds. Amino acids without a backbone hydrogen bond donor (proline, D-proline, N-methylated amino acids) are marked by darker columns.

FIG. 13. Structure-energy landscapes and hydrogen-bonding patterns for designed and selected 10-amino acid macrocycles. Left panels show the torsion bin cluster and predicted conformational landscape for each design. * denotes N-methylated amino acids. Points denote an independently predicted structure with no cis-peptide bonds. Points represent predicted structures with at least one cis-peptide bond. Dots represent the local minimization of the designed macrocycle structure. For hydrogen bonding graphs, the boxes highlight the designed intramolecular hydrogen bonds. Amino acids without a backbone hydrogen bond donor (proline, D-proline, N-methylated amino acids) are marked by darker columns.

FIG. 14. Structure-energy landscapes and hydrogen-bonding patterns for designed and selected 11-amino acid macrocycles. Left panels show the torsion bin cluster and predicted conformational landscape for each design. * denotes N-methylated amino acids. Points denote an independently predicted structure with no cis-peptide bonds. Points represent predicted structures with at least one cis-peptide bond. Dots represent the local minimization of the designed macrocycle structure. For hydrogen bonding graphs, the boxes highlight the designed intramolecular hydrogen bonds. Amino acids without a backbone hydrogen bond donor (proline, D-proline, N-methylated amino acids) are marked by darker columns.

FIG. 15. Structure-energy landscapes and hydrogen-bonding patterns for designed and selected 12-amino acid macrocycles: Left panels show the torsion bin cluster and predicted conformational landscape for each design. * denotes N-methylated amino acids. Points denote an independently predicted structure with no cis-peptide bonds. Points represent predicted structures with at least one cis-peptide bond. Dots represent the local minimization of the designed macrocycle structure. For hydrogen bonding graphs, the boxes highlight the designed intramolecular hydrogen bonds. Amino acids without a backbone hydrogen bond donor (proline, D-proline, N-methylated amino acids) are marked by darker columns.

FIG. 16. Designs with exposed NH groups show no permeability or low permeability in PAMPA.

FIG. 17. Superposition between the designed structure and X-ray structure of three closely-related cis-peptide bond containing designs, D8.13 (left panel), D8.14 (middle panel), and D8.15 (right panel). All three design models match closely (RMSD over all backbone atoms (N, CA, C, O, CN)<1.0 Å) with respective X-ray structures. All three design models feature a cis-peptide bond in the validated structures. However, the D8.13 is not permeable in PAMPA, while both D8.14 and D8.15 show significant permeability (Pₐₚₚ>1×10⁻⁶ cm/s), indicating that cis-peptide bond alone is not enough to drive permeability in these macrocycles.

FIG. 18. Cis-trans isomerization of the peptide bonds generates alternative low-energy states: (A) Structure prediction calculations for the design D11.25 sequence shows two low-energy states. Points: conformations with no cis-peptide bonds; points: predicted conformations with at least one cis-peptide bonds; and points: conformations generated by the local energy minimization of the design model. (B) Lowest energy “trans” state with all peptide bonds in trans conformation, (C) Lowest energy ‘cis’ state with the N-methylated amino acid in cis conformation. (D) X-ray structure for D11.25 matches the cis-state and exposed NH group from a D-leucine. The position of cis peptide bond is highlighted in the dashed square. *in the cis state denotes the position of the unsatisfied NH groups.

FIG. 19. Low-energy structural clusters for design D8.31: (A) The 250 lowest energy predicted structures for D8.31 were selected and clustered using the Rosetta™ energy_based_clustering application. The cluster naming (LE_X) is based on the ranking of the lowest-energy member from each cluster. The lowest energy structure from each identified cluster is labeled on the Energy vs. RMSD plot. Points: predicted structures with no cis-peptide bond; points: predicted structures with at least one cis-peptide bond. (B) Lowest energy member from each cluster is shown in the stick representation. Position of cis peptide bonds indicated in labels. Sidechains for non-proline (or D-proline) positions are not shown for clarity. The boxes with grey background denote the structures that match the X-ray crystal or the NMR structures.

FIG. 20. Low-energy structural clusters for design D8.21: (A) The 500 lowest energy predicted structures for D8.21 were selected and clustered using the Rosetta™ energy_based_clustering application. The cluster naming (LE_X) is based on the ranking of the lowest-energy member from each cluster. The lowest energy structure from each identified cluster is labeled on the Energy vs. RMSD plot. Points: predicted structures with no cis-peptide bond; points: predicted structures with at least one cis-peptide bond; points: structures obtained after local minimization of the design model (LE_0) (B) Lowest energy member from each cluster is shown in the stick representation. The representation is based on the presence or absence of any cis-peptide bond in the structure. Position of cis peptide bonds indicated in labels. Sidechains for non-proline (or D-proline) positions are not shown for clarity. The boxes with grey background denote the structures that match the X-ray crystal or the NMR structures.

FIG. 21. Low-energy structural clusters for design D9.16: (A) The 250 lowest energy predicted structures for D9.16 were selected and clustered using the Rosetta™ energy_based_clustering application. The cluster naming (LE_X) is based on the ranking of the lowest-energy member from each cluster. The lowest energy structure for 10 lowest-energy clusters is labeled on the Energy vs. RMSD plot. Points: predicted structures with no cis-peptide bond; points: predicted structures with at least one cis-peptide bond; points: structures obtained after local minimization of the design model (LE_0) (B) Lowest energy member from each of the 10 lowest-energy clusters is shown in the stick representation. Representation is based on the presence or absence of any cis-peptide bond in the structure. Position of cis peptide bonds indicated in labels. Sidechains for non-proline (or D-proline) positions are not shown for clarity. The boxes with grey background denote the structures that match the X-ray crystal or the NMR structures.

### DETAILED DESCRIPTION

All references cited are herein incorporated by reference in their entirety.

As used herein, the singular forms “a”, “an” and “the” include plural referents unless the context clearly dictates otherwise. “And” as used herein is interchangeably used with “or” unless expressly stated otherwise.

As used herein, the amino acid residues are abbreviated as follows: alanine (Ala; A), asparagine (Asn; N), aspartic acid (Asp; D), arginine (Arg; R), cysteine (Cys; C), glutamic acid (Glu; E), glutamine (Gln; Q), glycine (Gly; G), histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), valine (Val; V), and alpha-aminoisobutyric acid (AIB, B). Amino acid residues in D-form are noted with a “D” preceding the amino acid residue abbreviation. Amino acid residues in L-form are noted with just the amino acid residue abbreviation, noting that Glycine and alpha-aminoisobutyric acid are non-chiral.

All embodiments of any aspect of the invention can be used in combination, unless the context clearly dictates otherwise.

In a first aspect, the disclosure provides cyclic peptides comprising or consisting of an amino acid sequence having no more than 3 amino acid substitutions compared to the amino acid sequence selected from the group consisting of SEQ ID NO: 1-123 and 125-131, or mirror images thereof, wherein residues in uppercase are L amino acids, and residues in lower case are D amino acids, wherein an asterisk identifies the amino acid residue following the asterisk as being an N-methylated amino acid. The amino acid sequences of SEQ ID NO: 1-123 and 125-131 are shown in Table 1.

TABLE 1

| SEQ ID NO | |
|---|---|
| | 6-mers |
| 1 | iPfiPf |
| 2 | pfaPPl |
| 3 | p*faPPl |
| 4 | PPVfPf |
| 5 | PP*VfPf |
| 6 | *faP*vFP |
| 7 | vf*LpFP |
| 8 | *Vf*LpFP |
| 9 | iP*fiP*f |
| 10 | faPvFP |
| | 7-mers |
| 11 | P*lvLPpI |
| 12 | lp*LIP*lL |
| 13 | LP*llPpl |
| 14 | lp*ALpPL |
| 15 | VIpFIpa |
| 16 | Pplf*Lvf |
| 17 | VIp*FIpa |
| | 8-mers |
| 18 | lLplLaPL |
| 19 | lvP*lliP*l |
| 20 | FLPplVpl |
| 21 | LPpLv*a*al |
| 22 | P*lliP*liv |
| 23 | pp*AfA*L*ll |
| 24 | lVpPlVpP |
| 25 | Plf*FP*llp |
| 26 | Plf*FP*ll*G |
| 27 | plVpp*LvI |
| 28 | P*llFpp*aI |
| 29 | v*l*A*avPpI |
| 30 | PpA*L*lIvP |
| 31 | pp*LlpP*ll |
| 129 | pP*llpP*ll |
| 32 | PBliPBli |
| 33 | ia*A*lPv*lP |
| 34 | f*a*Lpllpv |
| 35 | Pa*l*f*Lvap |
| 36 | V*LpLV*LpL |
| 37 | APpVA*L*lV |
| 38 | lV*l*llVpP |
| 39 | lp*la*lplA |
| 40 | Fpp*LIPpl |
| 41 | MpPll*avP |
| 42 | ap*LLP*lv*a |
| 43 | ap*lvap*lv |
| 44 | fiPpivP*f |
| 47 | FpPL*l*I*IL |
| 56 | pP*lIpP*Il |
| | 9 mers |
| 45 | pPBLBVP*lI |
| 46 | Lip*LlP*fVl |
| 48 | Pv*avPlv*Lf |
| 49 | Pav*L*FIAPl |
| 50 | *LvL*L*fvpvp |
| 51 | PlLLP*a*aIp |
| 52 | pA*F*la*lv*VP |
| 53 | A*l*lPlpp*lf |
| 54 | P*aV*l*llL*aP |
| 55 | v*A*A*l*lfppP |
| 57 | Vf*a*L*lIpPa |
| 58 | piIF*FPp*LI |
| 59 | PlI*avpPlP |
| 60 | PpPaI*l*lfa |
| 61 | P*aaV*lllpL |
| 62 | APpVMPv*l*a |
| 63 | p*aV*l*apPLP |
| 64 | i*aiPAvpPl |
| 65 | piPP*lvI*la |
| 66 | lLp*LlP*aMa |
| 67 | vpPa*l*laVp |
| 68 | plAP*aLi*lV |
| 69 | lVI*I*FVPGP |
| 70 | pAllp*FLvP |
| 71 | pPGIlVP*aI |
| 72 | pMlP*lLF*lV |
| 73 | PpAP*laFif |
| 74 | pIl*lp*FvVP |
| 92 | vV*LPVL*Lpl |
| 94 | Pv*L*f*fp*LiA |
| 130 | pv*M*alpPLP |
| | 10-mers |
| 75 | Ip*zFap*Lv*lPv |
| 76 | PpAv*LA*lp*VV |
| 77 | lL*LLP*LA*AlP |
| 78 | *L*FLp*LiPpAv |
| 79 | aLVp*LVP*lV*I |
| 80 | P*lAP*lfLV*L*L |
| 81 | fA*l*A*LPpViP |
| 82 | Pi*A*LLPpA*Lf |
| 83 | l*ApppPv*L*lA |
| 84 | FpPvV*l*a*Vpa |
| 85 | P*lFiPLlp*av |
| 86 | F*alIPpVP*al |
| 87 | P*af*LlpPa*LV |
| 88 | fip*A*lLLpPl |
| 89 | l*aLfipaPpL |
| 90 | M*a*L*alpapPL |
| 91 | vlpvpPp*Lf*L |
| 92 | *G*FLp*LiPpAv |
| 93 | vafI*l*aPpPM |
| 94 | *L*fLp*LiPpAv |
| 95 | aP*fpFpPALL |
| 96 | aGpf*F*FfafP |
| 97 | PvpVVPvpFF |
| 98 | Llp*ApP*lA*lV |
| 99 | P*a*lAlPpi*li |
| 100 | p*lvIp*ApL*ll |
| 101 | apPvl*l*lAlP |
| 102 | PviP*a*Lplli |
| 103 | aPVP*faAV*fL |
| 104 | LVf*Ip*FlPLp |
| 105 | *L*GP*la*LP*ILF |
| 106 | *f*LPAV*I*FI*A*A |
| 107 | lILp*FVP*GF*L |
| 108 | LfPla*LL*AfP |
| | 11-mers |
| 109 | VPpL*iip*LIip |
| 110 | VPpL*iip*FIip |
| 111 | AP*lVliP*l*Ap*L |
| 112 | V*L*lAipPf*a*lP |
| 113 | V*l*LplapPAfl |
| 114 | pI*AipPlI*Av*L |
| 115 | Ppp*lf*L*lIAPv |
| 116 | PpVv*llpPI*AI |
| 118 | Lpp*LL*alp*la*l |
| 119 | Lf*L*app*lvPpa |
| 120 | P*aLPp*a*lF*lVI |
| 121 | PI*V*apPlV*lfp |
| 122 | vp*vl*IIP*liIP |
| 123 | ipLlp*LfL*aiP |
| 125 | lp*fapPpaall |
| 126 | ipLlp*LfL*giP |
| | 12-mers |
| 127 | p*FIpLlLpPa*aI |
| 128 | p*VIpLfLpPa*lI |
| 131 | VP*liPVAP*VlP*a |
| 117 | VP*liPVAP*vlP*a |

The cyclic peptides of the disclosure are shown in the attached appendices to possess membrane permeability and oral bioavailability. The cyclic peptides can be used, for example, to traverse membranes and target proteins and protein-protein interfaces, and label other biomolecules and transport them into the cells. Mirror images of the cyclic peptides will have the structure with mirror handedness and the same level of permeability. By way of non-limiting example, the mirror image of SEQ ID NO: 1 (iPfiPf) is IpFIpE (SEQ ID NO: 132).

As used herein, the cyclic peptides are 6 to 12 amino acids in length. In various embodiments, the cyclic peptide may be 6-12, 6-11, 6-10, 6-9, 6-8, 6-7, 7-12, 7-11, 7-10, 7-9, 7-8, 8-12, 8-11, 8-10, 8-9, 9-12, 9-11, 9-10, 10-12, 10-11, 11-12, 6, 7, 8, 9, 10, 11, or 12, amino acids in length. The cyclic peptides of the disclosure are N-to-C cyclized.

The cyclic peptides may be linked to other moieties (linkers, dyes, purification tags, peptides, small molecules, nucleic acids, etc.) as deemed appropriate for an intended use. Conjugation of one or more these membrane-traversing cyclic peptide to such other moieties (cargo) for delivery into the cell. The conjugation cab be performed, for example, through the side chains of the membrane-traversing peptide.

In one embodiment, the cyclic peptide comprises or consists of an amino acid sequence having no more than 3 amino acid substitutions compared to the amino acid sequence selected from the group consisting of SEQ ID NO: 1-128 or mirror images thereof, wherein residues in uppercase are L amino acids, and residues in lower case are D amino acids, wherein an asterisk identifies the amino acid residue following the asterisk as being an N-methylated amino acid.

In another embodiment, the cyclic peptide comprises or consists of an amino acid sequence having no more than 3 amino acid substitutions compared to the amino acid sequence selected from the group consisting of SEQ ID NO:1-46, 48-55, 57-68, 70-91, 93, 96-103, 109-116, 118-123, 125, and 127-128, or mirror images thereof, wherein residues in uppercase are L amino acids, and residues in lower case are D amino acids, wherein an asterisk identifies the amino acid residue following the asterisk as being an N-methylated amino acid.

In another embodiment, the cyclic peptide comprises or consists of an amino acid sequence having no more than 3 amino acid substitutions compared to the amino acid sequence selected from the group consisting SEQ ID NO: 19, 36, 43, 61, 109, or mirror images thereof, wherein residues in uppercase are L amino acids, and residues in lower case are D amino acids, wherein an asterisk identifies the amino acid residue following the asterisk as being an N-methylated amino acid.

In one embodiment, the cyclic peptides are cell membrane permeable. As shown in the examples, the inventors designed a wide diversity of membrane-permeable macrocycles. In another embodiment, the cyclic peptides have an apparent permeability greater than 1×10⁻⁷ cm/s. In a further embodiment, the cyclic peptides have an apparent permeability greater than 1×10⁻⁶ cm/s. In one embodiment, apparent permeability is measured by rates of traversal across artificial membranes in parallel artificial membrane permeability assays (PAMPA)¹⁸ were determined by mass spectrometry-based quantification of peptide concentrations in the donor and acceptor wells. In a further embodiment, the apparent permeability is measured using the PAMPA assay as detailed in the Supplementary Materials.

TABLE 2

| | | | | #_methyl | | |
|---|---|---|---|---|---|---|
| SEQ ID | | | Bin_ | yated | PAMPA_ | PAMPA_ |
| NO | Name | Sequence | String | AAs | Papp_Avg | PaPP_Stdev |
| 1 | D6.1 | iPfiPf | ABYABY | 0 | 3.34E−06 | 8.88E−07 |
| 2 | D6.3 | pfaPpl | AAAYYB | 0 | 1.99E−06 | 2.82E−06 |
| 3 | D6.4 | p*faPPl | *ABYYBB | 1 | 1.39E−05 | 5.74E−06 |
| 4 | D6.5 | PPVfPf | AAYBYB | 0 | 2.41E−06 | 1.27E−06 |
| 5 | D6.6 | PP*VfPf | A*AYBYB | 1 | 3.64E−06 | 2.74E−06 |
| 6 | D6.8.p1 | *faP*vF | *ABY*AYY | 2 | 3.47E−06 | 6.60E−07 |
| | | P | | | | |
| 6 | D6.8.p2 | *faP*vF | *ABY*AYY | 2 | 1.77E−06 | 2.68E−07 |
| | | P | | | | |
| 7 | D6.9.p1 | vf*LpFP | AB*YBYY | 1 | 5.29E−06 | 1.52E−06 |
| 7 | D6.9.p2 | vf*LpFP | AB*YBYY | 1 | 6.77E−06 | 1.48E−06 |
| 8 | D6.10 | *vf*LpF | *AB*YBYY | 2 | 1.16E−05 | 5.68E−07 |
| | | P | | | | |
| 9 | D6.2 | iP*fiP* | *ABY*ABY | 2 | 1.22E−05 | 4.73E−06 |
| | | f | | | | |
| 10 | D6.7 | faPvFP | ABYAYX | 0 | 4.24E−07 | 1.81E−07 |
| 11 | D7.1 | P*lvLPpI | *AAYYAYY | 1 | 2.93E−06 | 5.41E−07 |
| 12 | D7.2 | lp*LIP*l | *ABB*YAY | 2 | 1.165−05 | 4.56E−06 |
| | | L | Y | | | |
| 13 | D7.5 | LP*llPpl | AAYY*ABY | 1 | 6.64E−06 | 1.19E−07 |
| 14 | D7.6 | lp*ALpPL | AAYY*BAY | 1 | 3.62E−06 | 2.18E−06 |
| 15 | D7.8.p1 | VIpFIpa | AAXYBXY | 0 | 4.87E−06 | 1.04E−06 |
| 15 | D7.8.p2 | VIpFIpa | AAXYBXY | 0 | 4.92E−06 | 6.26E−07 |
| 16 | D7.7.p1 | Pplf*Lvf | ABB*XABY | 1 | 8.43E−06 | 7.80E−07 |
| 16 | D7.7.p2 | Pplf*Lvf | ABB*XABY | 1 | 9.06E−06 | 2.30E−06 |
| 17 | D7.9 | VIp*FIpa | AAXYB*XY | 1 | 7.09E−06 | 8.09E−07 |
| 18 | D8.1 | lLplLaPL | AAYBXXB | 0 | 6.94E−06 | 4.70E−07 |
| | | | Y | | | |
| 19 | D8.3.p1 | lvP*lliP | *AABY*A | 2 | 4.61E−06 | 6.73E−07 |
| | | *l | ABY | | | |
| 129 | D8.4 | pP*llpP* | *ABBY*B | 2 | 3.58E−08 | 1.15E−08 |
| | | ll | BBY | | | |
| 20 | D8.5 | FLPplVpl | AAXYYAA | 0 | 6.31E−06 | 1.63E−06 |
| | | | Y | | | |
| 21 | D8.6 | LPpLv*a* | *A*AXBB | 2 | 3.35E−06 | 2.45E−07 |
| | | al | YAY | | | |
| 22 | D8.8 | P*lliP*l | *AABY*A | 2 | 5.90E−06 | 3.21E−06 |
| | | iv | ABY | | | |
| 23 | D8.10 | pp*AfA*L | *ABBB*X | 3 | 1.23E−06 | 4.83E−07 |
| | | *ll | AY*Y | | | |
| 24 | D8.12 | lVpPlVpP | AYBYAYB | 0 | 7.87E−07 | 1.38E−07 |
| | p1 | | Y | | | |
| 24 | D8.12. | lVpPlVpP | AYBYAYB | 0 | 2.00E−07 | 2.42E−08 |
| | p2 | | Y | | | |
| 25 | D8.14 | Plf*FP*l | AAY*OB* | 2 | 3.68E−07 | 2.55E−07 |
| | | lp | YYY | | | |
| 26 | D8.15 | Plf*FP*l | AAY*OB* | 2 | 7.68E−07 | 2.47E−07 |
| | | l*G | YYY | | | |
| 27 | D8.16 | plVpp*Lv | AAYBB*Y | 1 | 2.16E−06 | 6.19E−07 |
| | | I | AY | | | |
| 28 | D8.17 | P*llFpp* | *AAYBA* | 2 | 9.50E−07 | 1.82E−07 |
| | | aI | AYY | | | |
| 29 | D8.26 | v*l*A*av | *A*B*XY | 3 | 5.44E−07 | 5.90E−08 |
| | | PpI | BYAY | | | |
| 30 | D8.28 | PpA*L*lI | *AXAYYA | 2 | 7.10E−07 | 2.95E−07 |
| | | vP | YAY | | | |
| 31 | D8.32 | pp*LlpP* | *ABBB*X | 2 | 5.23E−06 | 3.93E−07 |
| | | ll | BBY | | | |
| 32 | D8.2 | PBliPBLi | AAXYAAXY | 0 | 7.49E−06 | 2.80E−06 |
| 33 | D8.18 | ia*A*lPv | AA*Y*BXB | 3 | 1.04E−06 | 2.34E−07 |
| | | *lP | *BX | | | |
| 34 | D8.19 | f*a*Lpll | AAAAAA*B | 2 | 6.26E−07 | 1.49E−07 |
| | | pv | *Y | | | |
| 35 | D8.20 | Pa*l*f*L | ABBXB*B* | 3 | 1.08E−06 | 3.46E−07 |
| | | vap | B*X | | | |
| 36 | D8.21 | v*LpLv*L | AXB*YAXB | 2 | 2.03E−06 | 4.72E−07 |
| | | pL | *Y | | | |
| 37 | D8.22 | APpVA*L* | AB*B*XAB | 2 | 2.15E−07 | 5.13E−08 |
| | | lV | BX | | | |
| 38 | D8.23 | lV*l*llV | *A*AAYBX | 2 | 1.28E−06 | 1.11E−07 |
| | | pP | AY | | | |
| 39 | D8.25 | lp*la*lp | *AAAXBA* | 2 | 8.24E−07 | 9.75E−08 |
| | | lA | AB | | | |
| 40 | D8.27 | Fpp*LIPp | AAXBB*YY | 1 | 3.15E−07 | 3.83E−09 |
| | | l | Y | | | |
| 41 | D8.29 | MpPll*av | AY*YBYYB | 1 | 2.36E−07 | 4.97E−08 |
| | | P | Y | | | |
| 42 | D8.30 | ap*LLP*l | A*AY*YYY | 3 | 4.32E−06 | 2.61E−07 |
| | | v*a | *BB | | | |
| 43 | D8.31 | ap*lvap* | A*AABB*X | 2 | 2.47E−06 | 6.99E−07 |
| | | lv | AY | | | |
| 44 | D8.33 | fiPpivP* | AABY*AAB | 1 | 6.00E−06 | 1.99E−06 |
| | | f | Y | | | |
| 45 | D9.1 | pPBLBVP* | *AYBYAYB | 1 | 1.59E−06 | 1.50E−07 |
| | | lI | YY | | | |
| 46 | D9.3 | Lip*LlP* | *AXBYBB* | 2 | 2.33E−06 | 6.38E−07 |
| | | fVl | XBY | | | |
| 48 | D9.9 | Pv*avPlv | *AYBY*YY | 2 | 3.51E−06 | 8.27E−07 |
| | | *Lf | BYY | | | |
| 49 | D9.6 | Pav*L*FI | *A*AABBY | 2 | 1.63E−06 | 2.96E−07 |
| | | APl | BYY | | | |
| 50 | D9.8.p1 | *LvL*L*f | *ABBBB*Y | 3 | 8.64E−07 | 9.35E−07 |
| | | vpvp | AY*Y | | | |
| 50 | D9.8.p2 | *LvL*L*f | *ABBBB*Y | 3 | 2.55E−07 | 1.42E−07 |
| | | vpvp | AY*Y | | | |
| 51 | D9.11 | PlLLP*a* | *A*AYBYA | 2 | 3.16E−07 | 4.54E−08 |
| | | aIp | XYY | | | |
| 52 | D9.17 | pA*F*la* | *AA*XA*Y | 4 | 1.74E−06 | 1.23E−06 |
| | | lv*VP | YAY*Y | | | |
| 53 | D9.18 | A*l*lPlp | A*AAX*B* | 3 | 1.76E−06 | 2.82E−07 |
| | | p*lf | BXBB | | | |
| 54 | D9.19 | P*aV*l*l | *A*AAX*Y | 4 | 1.28E−06 | 1.69E−07 |
| | | lL*aP | YY*AY | | | |
| 130 | D9.24 | pv*M*alp | *ABBXYYB | 2 | 8.69E−08 | 9.47E−09 |
| | | PLP | A*Y | | | |
| 55 | D9.25 | V*A*A*l* | AY*B*B*Y | 4 | 2.19E−06 | 6.23E−07 |
| | | lfppP | *AYYY | | | |
| 57 | D9.10 | Vf*a*L*l | AA*Y*B*X | 3 | 8.26E−07 | 3.25E−07 |
| | | IpPa | BYBX | | | |
| 58 | D9.13 | piIF*FPp | AAXY*ZYB | 2 | 3.37E−06 | 2.72E−07 |
| | | *LI | *XY | | | |
| 59 | D9.14 | PlI*avpP | ABBBXB*Y | 1 | 4.21E−07 | 1.53E−08 |
| | | lP | YY | | | |
| 60 | D9.15 | PpPaI*l* | *A*AABYB | 2 | 3.41E−07 | 8.75E−08 |
| | | lfa | YAY | | | |
| 61 | D9.16 | P*aaV*ll | *AABBYX* | 2 | 3.27E−06 | 8.50E−07 |
| | | lpL | XBY | | | |
| 62 | D9.21 | APpVMPv* | *A*AYYAX | 2 | 2.94E−07 | 1.40E−08 |
| | | l*a | YYB | | | |
| 63 | D9.22 | p*aV*l*a | A*AY*B*B | 3 | 1.89E−07 | 8.37E−09 |
| | | pPLP | BXYY | | | |
| 64 | D9.23 | i*aiPAVp | AAYYBXB* | 1 | 1.41E−07 | 7.42E−09 |
| | | Pl | AY | | | |
| 65 | D9.26 | piPP*lvI | *ABBAYY* | 2 | 1.99E−06 | 2.78E−07 |
| | | *la | AYY | | | |
| 66 | D9.28 | lLp*LlP* | A*AAXXBY | 2 | 1.57E−06 | 2.23E−07 |
| | | aMa | *AY | | | |
| 67 | D9.29 | vpPa*l*l | *A*AAYAB | 2 | 6.64E−07 | 5.78E−08 |
| | | aVp | BXB | | | |
| 68 | D9.30 | plAP*aLi | A*AAX*XB | 2 | 1.60E−06 | 3.92E−08 |
| | | *lV | XYB | | | |
| 70 | D9.32 | pAllp*FL | *AAYBYAX | 1 | 2.40E−07 | 4.31E−08 |
| | | vP | YY | | | |
| 71 | D9.33 | pPGIlVP* | *AYBYAYB | 1 | 3.17E−07 | 1.40E−07 |
| | | aI | YY | | | |
| 72 | D9.40 | pMlP*lLF | AB*YBYBZ | 2 | 2.38E−07 | 1.56E−07 |
| | | *lV | B*X | | | |
| 73 | D9.41 | PpAP*laF | ABYAZY*B | 1 | 1.02E−06 | 7.22E−08 |
| | | if | BX | | | |
| 74 | D9.42 | pIl*lp*F | AXB*OB*Y | 2 | 1.12E−06 | 5.51E−08 |
| | | vVP | AYY | | | |
| 75 | D10.3. | Ip*Fap*L | AXBY*AYY | 3 | 2.52E−06 | 6.08E−07 |
| | | v*lPv | *AY*Y | | | |
| 76 | D10.6 | PpAv*LA* | *AB*YYYB | 3 | 5.09E−07 | 1.42E−07 |
| | | lp*VV | XB*YY | | | |
| 77 | D10.16 | lL*LLP*L | A*AB*BYB | 3 | 2.27E−06 | 6.75E−08 |
| | | A*AlP | YB*BB | | | |
| 78 | D10.17 | *L*FLp*L | *A*ABY*A | 3 | 2.69E−06 | 8.06E−07 |
| | | iPpAv | YBYAY | | | |
| 79 | D10.19 | aLVp*LVP | ABY*BBB* | 3 | 6.36E−06 | 1.65E−06 |
| | | *lV*I | YB*BX | | | |
| 80 | D10.20 | P*lAP*lf | *A*BB*XB | A | 2.59E−06 | 7.21E−07 |
| | | LV*L*L | B*XYBB | | | |
| 81 | D10.25 | fA*l*A*L | AAA*Y*B* | 3 | 7.56E−07 | 5.01E−07 |
| | | PpViP | BBXAY | | | |
| 82 | D10.26 | Pi*A*LLP | *ABBXB*B | 3 | 8.08E−07 | 1.74E−07 |
| | | pA*Lf | YBX*B | | | |
| 83 | D10.28 | l*ApppPv | *A*AAY*B | 3 | 1.84E−07 | 4.36E−08 |
| | | *L*lA | YYYAY | | | |
| 84 | D10.29 | FpPvV*l* | AXB*Y*Y* | 3 | 4.41E−07 | 4.66E−08 |
| | | a*Vpa | BYYBY | | | |
| 85 | D10.30 | P*lFiPLl | AAYY*AYB | 2 | 4.78E−06 | 6.39E−07 |
| | | p*av | *XAY | | | |
| 86 | D10.31 | F*alIPpV | *AAY*AAY | 2 | 1.90E−06 | 2.65E−07 |
| | | P*al | YAYY | | | |
| 87 | D10.32 | P*af*Llp | *AB*YBBX | 3 | 1.86E−06 | 6.20E−07 |
| | | Pa*LV | B*YYY | | | |
| 88 | D10.34 | fip*A*lL | ABB*Y*AY | 2 | 4.51E−06 | 2.89E−07 |
| | | LpPl | YBXY | | | |
| 89 | D10.37 | l*aLfipa | *AAXYYYB | 1 | 6.54E−07 | 9.96E−08 |
| | | PpL | YAY | | | |
| 90 | D10.40 | M*a*L*al | AAB*Y*B* | 3 | 3.40E−07 | 4.87E−08 |
| | | papPL | YYYYY | | | |
| 91 | D10.42 | vlpvpPp* | ABBBBYB* | 2 | 7.10E−07 | 1.94E−07 |
| | | Lf*L | XB*X | | | |
| 93 | D10.45 | vafI*l*a | AAAY*B*B | 2 | 4.87E−07 | 9.92E−08 |
| | | PpPM | YBXX | | | |
| 96 | D10.62 | aGpf*F*F | AAOYABBB | 2 | 4.23E−06 | 4.30E−07 |
| | | fafP | *X*Y | | | |
| 97 | D10.27 | PvpVVPvp | ABBYYABB | 0 | 4.90E−07 | 4.20E−08 |
| | | FF | YY | | | |
| 98 | D10.35 | Llp*ApP* | A*AA*AAA | 3 | 2.79E−07 | 2.19E−07 |
| | | LA*lV | YY*BY | | | |
| 99 | D10.39 | P*a*lAlP | *A*AAYBX | 3 | 1.48E−06 | 2.05E−07 |
| | | pi*li | X*BYB | | | |
| 100 | D10.41 | p*lvIp*A | A*AAYB*Y | 3 | 5.69E−06 | 2.80E−07 |
| | | pL*ll | BY*BB | | | |
| 101 | D10.43 | apPvl*l* | *A*AYXXB | 2 | 1.61E−07 | 2.53E−08 |
| | | LAlP | BXAB | | | |
| 102 | D10.46 | PviP*a*L | AABBYABY | 2 | 3.88E−07 | 1.03E−07 |
| | | plli | *B*Y | | | |
| 103 | D10.48 | aPVP*faA | *AAYY*AX | 2 | 7.16E−07 | 1.02E−07 |
| | | V*fL | BYYY | | | |
| 109 | D11.3 | VPpL*iip | *AAYYBBX | 2 | 1.48E−06 | 4.09E−07 |
| | | *LIip | B*YYY | | | |
| 110 | D11.4 | VPpL*iip | *AAYYBBX | 2 | 1.84E−06 | 1.62E−07 |
| | | *FIip | B*YYY | | | |
| 111 | D11.5 | AP*lVliP | A*AAXYB* | 4 | 2.81E−06 | 2.83E−07 |
| | | *l*Ap*L | Y*BY*BB | | | |
| 112 | D11.6 | V*L*lAip | *A*AAYYA | 4 | 1.70E−06 | 4.01E−07 |
| | | Pf*a*lP | Y*Y*YAB | | | |
| 113 | D11.9. | V*l*Lpla | AABBXXAB | 2 | 8.79E−07 | 2.37E−08 |
| | p1 | pPAfl | Y*B*Y | | | |
| 113 | D11.9. | V*l*Lpla | AABBXXAB | 2 | 2.09E−06 | 3.75E−08 |
| | p2 | pPAfl | Y*B*Y | | | |
| 114 | D11.10 | pI*AipPl | A*YBY*YB | 3 | 1.795−06 | 5.435−07 |
| | | I*Av*L | BYAY*Y | | | |
| 115 | D11.11 | Ppp*lf*L | ABBYBYY* | 3 | 2.81E−06 | 2.83E−07 |
| | | *lIAPv | AY*B*X | | | |
| 116 | D11.13 | PpVv*llp | AB*BBBXA | 2 | 1.43E−06 | 2.49E−07 |
| | | PI*AI | Y*YYY | | | |
| 118 | D11.15 | Lpp*LL*a | *AB*YYY* | 4 | 1.29E−06 | 3.14E−07 |
| | | lp*la*l | AY*YAYY | | | |
| 119 | D11.16 | Lf*L*app | AX*B*YYY | 3 | 9.93E−07 | 9.42E−08 |
| | | *lvPpa | *AYBYY | | | |
| 120 | D11.20 | P*aLPp*a | *A*AB*XA | 4 | 1.10E−06 | 3.28E−07 |
| | | *lF*lVI | BB*XBBY | | | |
| 121 | D11.21 | PI*V*apP | AB*B*YYB | 3 | 2.06E−07 | 1.82E−08 |
| | | lV*lfp | XB*YYY | | | |
| 122 | D11.23 | vp*vl*II | *AAYYBB* | 3 | 2.70E−06 | 2.20E−06 |
| | .p1 | P*liIP | YB*YYY | | | |
| 122 | D11.23 | vp*vl*II | *AAYYBB* | 3 | 1.31E−06 | 8.61E−08 |
| | .p2 | P*llIP | YB*YYY | | | |
| 123 | D11.1 | ipLlp*Lf | *ABYBBXB | 2 | 3.48E−06 | 1.93E−07 |
| | | L*aiP | B*XAY | | | |
| 125 | D11.25 | lp*fapPp | AAAAABB* | 1 | 4.17E−07 | 1.84E−08 |
| | | aall | XBBY | | | |
| 131 | D12.5 | VP*liPVA | AABB*XYB | 3 | 1.78E−08 | 6.46E−08 |
| | | P*vlP*a | *XBB*XY | | | |
| 127 | D12.6 | p*FIpLIL | *ABXBXBY | 2 | 1.39E−06 | 1.05E−07 |
| | | pPa*aI | AX*YBY | | | |
| 128 | D12.3 | p*VIpLfL | *ABXBXBY | 2 | 7.89E−07 | 1.02E−07 |
| | | pPa*lI | AY*YBY | | | |
| 47 | | FpPL*l*l | *A*A*AXY | 3 | 1.27E−06 | 5.85E−07 |
| | | *lL | BXY | | | |
| 56 | D8.4 | pP*llpP* | *ABBY*BB | 2 | 1.36E−07 | 2.03E−08 |
| | | ll | BY | | | |
| 69 | | *lvl*l*F | *ABBBB*X | 3 | 9.46E−07 | 4.35E−07 |
| | | VPGP | AY*Y | | | |
| 92 | | *G*FLp*L | AY*B*BBY | 3 | 3.768−07 | 5.90E−08 |
| | | iPpAv | *AYBY | | | |
| 94 | | *L*fLp*L | *A*ABY*A | 3 | 2.69E−06 | 8.06E−07 |
| | | iPpAv | YBYAY | | | |
| 95 | D10.49 | aP*fpFpP | ABBBYB*Y | 1 | 1.70E−07 | 5.72E−08 |
| | | ALL | YBY | | | |
| 104 | D10.50 | LVf*lp*F | *ABBBYAB | 2 | 6.28E−07 | 3.04E−07 |
| | | IPLp | Y*YY | | | |
| 105 | D10.57 | *L*GP*la | *AA*YY*B | 5 | 3.05E−06 | 3.24E−07 |
| | | *LP*lLF | XY*Y*BY | | | |
| 106 | D10.56 | *f*LPAV* | AY*B*YB* | 6 | 9.02E−06 | 1.11E−07 |
| | | l*Fi*A*A | Y*Y*B*YY | | | |
| 107 | D10.1. | lILp*FVP | *ABB*XB* | 3 | 3.92E−06 | 7.29E−07 |
| | p1 | *GF*L | BXABY | | | |
| 108 | | LfPla*LL | AB*YY*YB | 2 | 3.92E−06 | 5.67E−07 |
| | | *AfP | YYBY | | | |
| 117 | | VP*liPVA | AABB*XYB | 3 | 1.00E−07 | 6.70E−08 |
| | | P*vlP*a | *XBB*XY | | | |

In one embodiment, the cyclic peptide has at least one proline residue, and in other embodiments has at least two, three, four, or more proline residues. In these embodiments, the one or more proline residues help conformationally constrain the cyclic peptide.

In another embodiment, the cyclic peptide has at least one N-methylated amino acid, and in other embodiments has at least two, three, four, or more N-methylated amino acids. In these embodiments, the one or more N-methylated amino acids helps limit exposed and unsatisfied hydrogen bond donors in the cyclic peptide.

In a further embodiment, the cyclic peptide has at least two proline and N-methylated amino acid residues in total, and in other embodiments has at least three, four, five, six, or more proline and N-methylated amino acid residues in total.

In some embodiments, the cyclic peptide has no N-methylated amino acids. Non-limiting examples of this embodiment include peptides having the amino acid sequence of SEQ ID NO: 1, 2, 4, 10, 15, 18, 20, 24, 32, and 97 as shown in Table 1, and mirror images thereof. In other embodiments, the cyclic peptide has 1 N-methylated amino acid. In a further embodiment, the cyclic peptide has 2 N-methylated amino acids.

In one embodiment, any N-methylated residues can only be substituted with (a) other N-methylated amino acids, (b) D-proline, or (d) L-proline. In other embodiments, any D-proline residues can only be substituted with an N-methylated D-amino acid residue. In a further embodiment, any L-proline residues can only be substituted with an N-methylated L-amino acid residue.

In one embodiment, peptide comprises at least 1, 2, 3, 4, 5, 6, or more D amino acids. In another embodiment, any D-amino acid residues can only be substituted with other D-amino acid residues. In a further embodiment, any L-amino acid residues can only be substituted with other L-amino acid residues.

In one embodiment of any of these embodiments, the cyclic peptide has no more than 2 amino acid substitutions compared to the reference amino acid sequence. In another embodiment, wherein the cyclic peptide has no more than 1 amino acid substitutions compared to the reference sequence. In a further embodiment, the cyclic peptide comprises or consists of the amino acid sequence of the reference sequence.

In a second aspect, the disclosure provides cyclic peptides comprising or consisting of a bin string as listed in Table 3, wherein:
A is a right-handed helical region;
B is a right-handed strand region;
X is a mirror image of A;
Y is a mirror image of B;
O are amino acids with phi <0 and cis peptide bond between the residue i and i+1; and
Z are amino acids with phi >0 and cis peptide bond between the residues i and i+1.

As described in the examples that follow, each residue of the designed cyclic peptides were assigned a torsion bin (A (right-handed helical region), B (right-handed strand region), X (mirror of A), Y (mirror of B), O (amino acids with phi <0 and cis peptide bond between the residue i and i+1), and Z (amino acids with phi >0 and cis peptide bond between the residue i and i+1)), and the resultant torsion bin strings (for example XYABOX for a six residue peptide) were clustered. Because the choice of starting residue is arbitrary in a cycle, and both passive membrane permeability and fold propensity are invariant to mirroring, clusters with bin strings that transform into each other under circular permutation or mirror inversion were combined, and members of the resulting non-degenerate clusters are represented in Table 2 by the lowest alphabetical order of the torsion bin string over all permutations and inversions. Thus, while the bin order in Table 2 is arrange in the lowest alphabetical order, those of skill in the art will understand the other permutations that are covered. By way of non-limiting example, the cyclic peptide of SEQ ID NO: 1 has a bin string of ABYABY as shown in Table 2. In this embodiment, the bin string of ABYABY is equivalent to BYABYA, YABYAB, ABYABY, BYABYAB, and YABYAB.

Each bin string represents a unique structure, and each of these structures is primed for permeability. Sequence changes within the constraints defined above will fit the same structural genus and will be permeable.

The cyclic peptides of this second aspect may include any embodiment or combination of embodiments as disclosed above for the first aspect of the disclosure.

The cyclic peptides of any aspect of disclosure can be made by any suitable technique, including but not limited to the methods disclosed in the examples that follow.

In another embodiment, the disclosure provides conjugates, comprising the cyclic peptide of any embodiment or combination of embodiments disclosed herein bound to a moiety. As described in the examples that follow, the cyclic peptides of the disclosure are membrane permeable and orally bioavailable, and thus may be conjugated to any moiety of interest for which such membrane permeability and/or oral bioavailability are useful. The cyclic peptides can be used, for example, to traverse membranes and target proteins and protein-protein interfaces, and label other biomolecules and transport them into the cells. In non-limiting embodiments, the moiety may comprise a therapeutic agent, a diagnostic agent, a marker, linkers, dyes, purification tags, peptides, small molecules, nucleic acids, etc.

In a further embodiment, the disclosure provides cyclic peptide library, comprising two or more cyclic peptides and/or conjugates according to any embodiment or combination of embodiments disclosed herein. In various embodiments, the library may comprise at least 5, 10, 25, 50, 75, 100, 150, 200, or more cyclic peptides and/or conjugates according to any embodiment or combination of embodiments disclosed herein. The libraries may be used, for example, to quickly screen for macrocycles that can bind to specific proteins and be permeable at the same time.

In another embodiment, the disclosure provides uses and methods for use of the cyclic peptide, conjugate, and/or cyclic peptide library of any embodiment or combination of embodiments disclosed herein. In one embodiment, the cyclic peptides and conjugates may be used, for example, to carry a linked moiety across a cell membrane. For example, the methods may comprise administering a small molecule therapeutic linked to the cyclic peptide to permit delivery of the therapeutic into cells to effect a desired treatment outcome. In another embodiment, the cyclic peptides and conjugates may be used, for example, as a scaffold for target-based drug design or to screen molecules of interest for binding to one or more of the cyclic peptides. In another embodiment, the cyclic peptide conjugates to a therapeutic or diagnostic may be administered orally, in view of the oral bioavailability of the cyclic peptides.

### EXAMPLES

### Summary

Cyclic peptides have considerable potential advantages as therapeutics since, unlike small molecules, they can disrupt protein-protein interactions, and, unlike proteins, they can traverse biological membranes. However, realizing these advantages has been complicated by the difficulty to date in robustly designing peptides with diverse shapes and sizes to be membrane permeable. Here we use computational design coupled with experimental characterization to systematically investigate the design principles for membrane permeability and oral bioavailability. We designed 188 6-12 residue macrocycles with a wide range of predicted structures containing non-canonical backbone modifications, and experimentally determined structures of 34; 28 are very close to the computational models with RMSDs as low as 0.2 Å, demonstrating considerable control over the structure for this class of compounds. With such control, we show that membrane permeability can be systematically achieved by ensuring all amide (NH) groups are engaged in internal hydrogen bonding interactions. 129 designs over the 6-12 residue size range cross membranes with an apparent permeability greater than 1×10⁻⁷ cm/s; 82 at rates greater than 1×10⁻⁶ cm/s. We further show that designs with exposed NH groups can be made membrane permeable through the design of an alternative isoenergetic fully hydrogen bonded state, validated by experimentally determined structures, that is favored in the lipid membrane. The most permeable of these compounds have oral bioavailability as high as 40% in rodent models.

### Study

Macrocyclic peptides have considerable potential as therapeutics due to their high metabolic stability, ability to target protein-protein interfaces with high potency, and ease of modular synthesis. Here we take advantage of the ability of computational design to specify macrocycle structure to systematically explore the determinants of passive membrane permeability. We use a design-build-test approach where we design peptides containing different structural features, determine their crystal structures, and evaluate their permeability. We consider three structural features: the satisfaction of all hydrogen bond donors through the formation of intrapeptide hydrogen bonds, the presence of cis-peptide bonds, and the ability to switch conformations between aqueous and membrane environments.

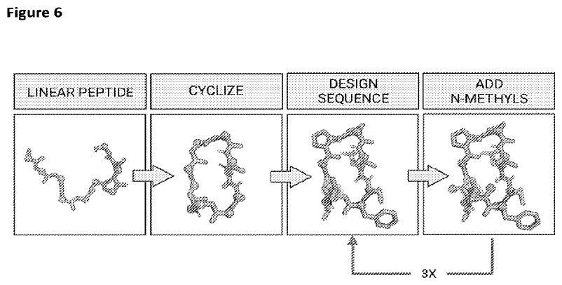

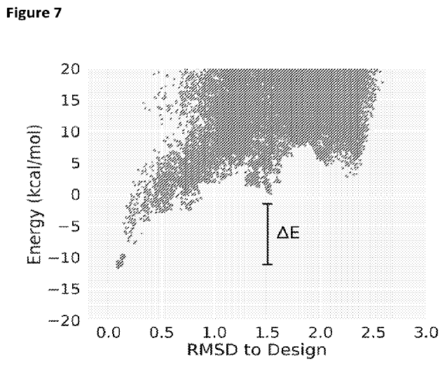

We first investigated whether designed macrocycles with widely diverse lengths and structures sharing only the property of full internal satisfaction of all NH groups could robustly traverse lipid membranes. We extended the Rosetta™ generalized kinematic closure (genKIC) method to stochastically generate ensembles of ~10⁶ N-to-C cyclic backbone conformations for 6-12 residue poly-glycine peptides, sampling cyclic backbone conformations by selecting phi/psi torsions randomly from flat-bottom symmetric Ramachandran tables^{16,17}. From these large sets, we selected backbones that make at least two intramolecular hydrogen bonds, and carried out Rosetta™ combinatorial sequence design restricting L- and D-amino acids to negative and positive phi regions of Ramachandran space, respectively, and incorporating conformationally-constrained amino acids, such as L-proline, D-proline, and α-aminoisobutyric acid (AIB), at structurally compatible sites. To eliminate exposed and unsatisfied hydrogen bond donors, amino acids with an unsatisfied hydrogen bond donor in the backbone were mutated to their N-methylated variants, and only non-polar amino acids were allowed during the sequence design step (see Supplementary Methods and FIG. 6). We selected low energy designs with two or more intramolecular hydrogen bonds and five or fewer N-methylated amino acids. The conformational energy landscape for selected designs was characterized by generating 10⁵ to 10⁶ alternative conformations and evaluating the energy and backbone RMSD to the design model. We chose sequences with funnel-like energy landscapes converging on their corresponding design models (see supplementary methods and FIG. 7). Overall, structural diversity was assessed using a backbone torsion angle-based clustering method¹⁶. Each residue was assigned a torsion bin (A (right-handed helical region), B (right-handed strand region), X (mirror of A), Y (mirror of B), O (amino acids with phi <0 and cis peptide bond between the residue i and i+1), and Z (amino acids with phi >0 and cis peptide bond between the residue i and i+1)), and the resultant torsion bin strings (for example XYABOX for a six residue peptide) were clustered. Because the choice of starting residue is arbitrary in a cycle, and both passive membrane permeability and fold propensity are invariant to mirroring, clusters with bin strings that transform into each other under circular permutation or mirror inversion were combined, and members of the resulting non-degenerate clusters (which we represent by the lowest alphabetical order of the torsion bin string over all permutations and inversions; see Table 2) were selected for chemical synthesis and experimental characterization.

### Membrane Permeability of 6-8 Residue Designed Macrocycles

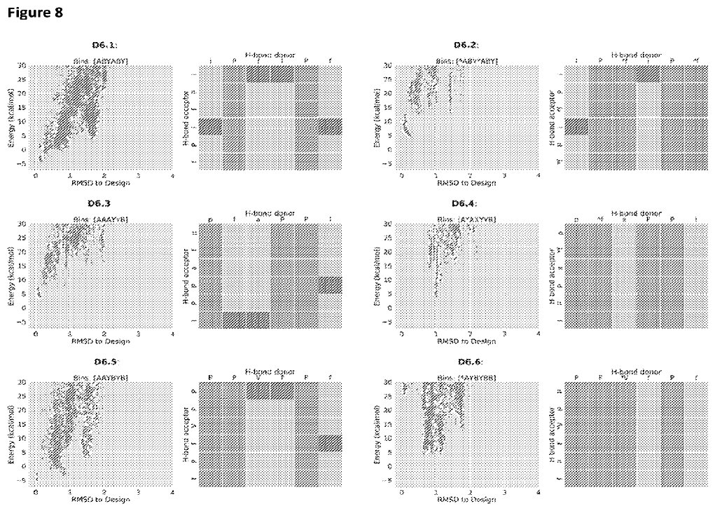

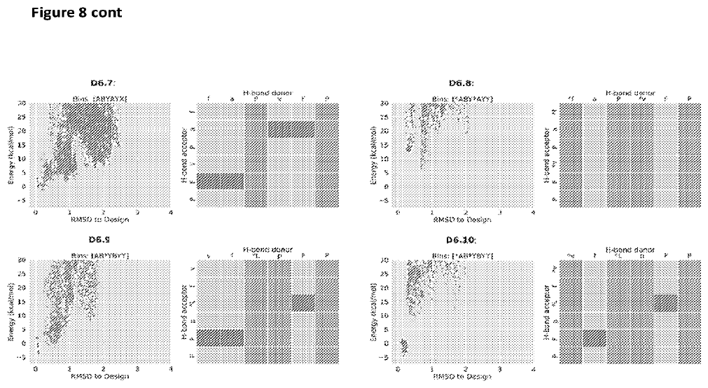

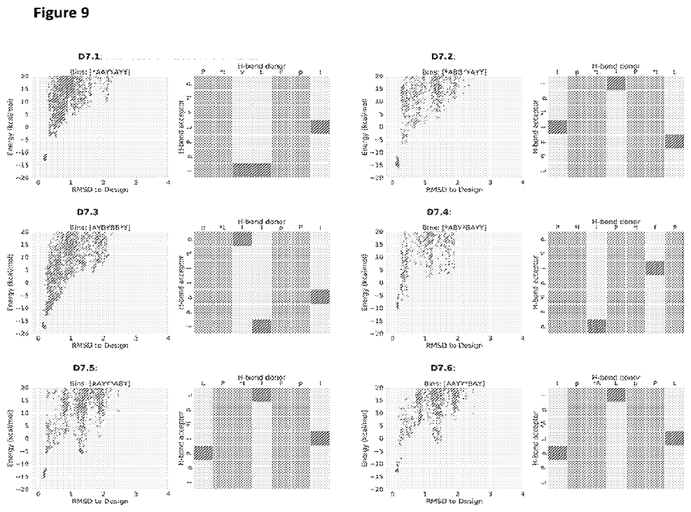

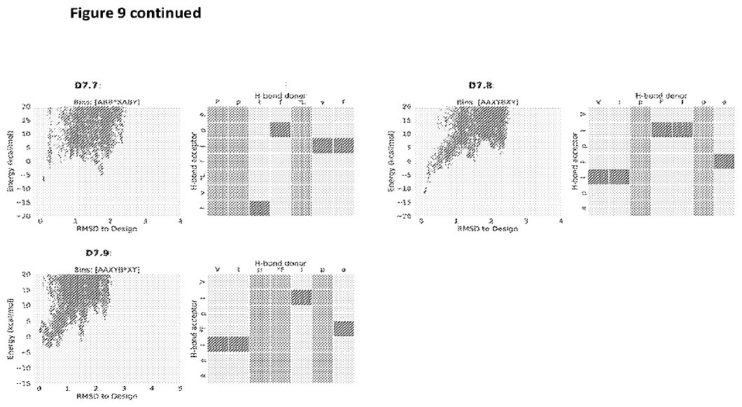

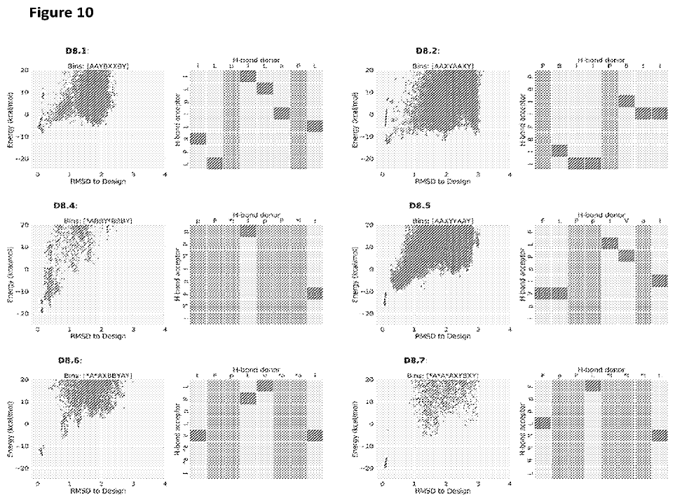

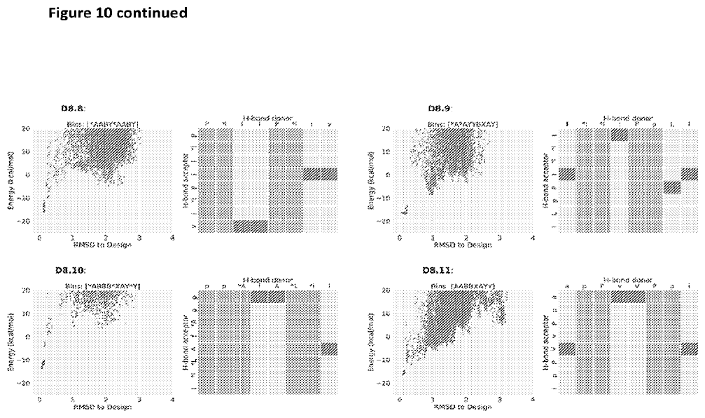

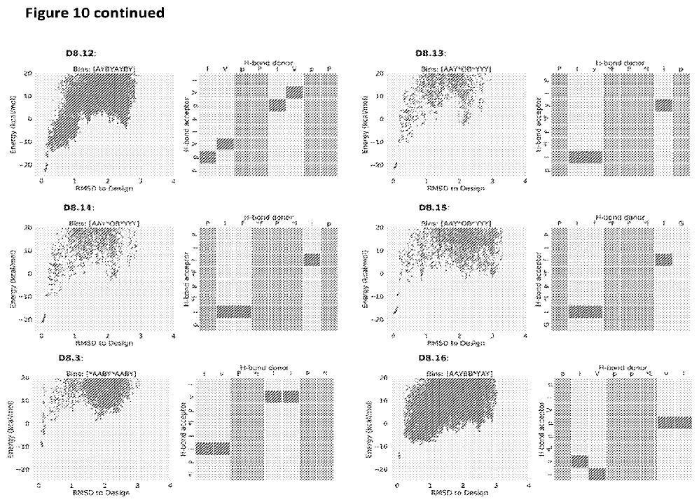

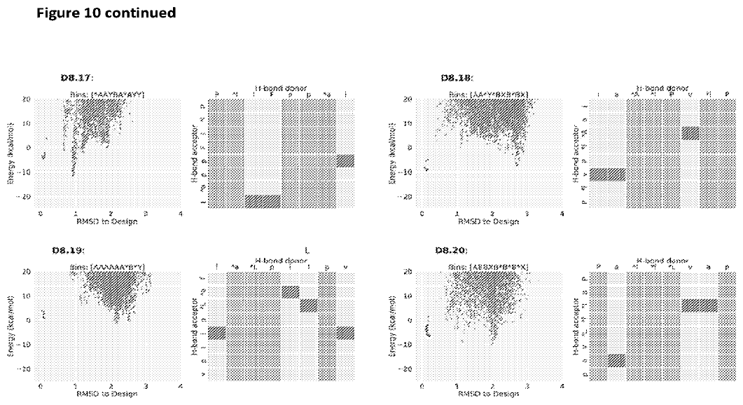

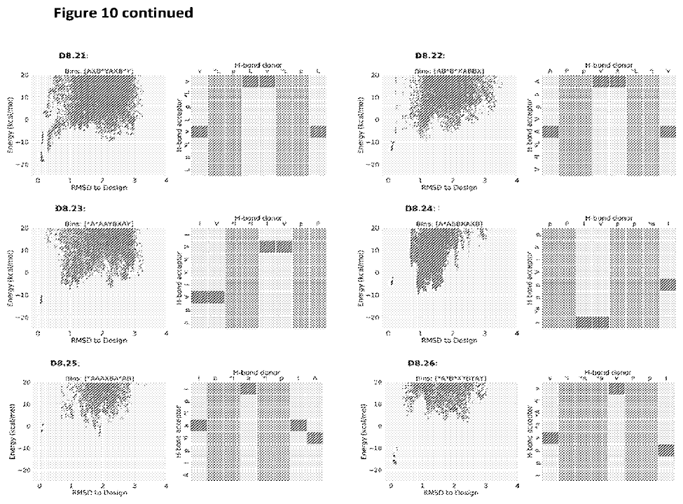

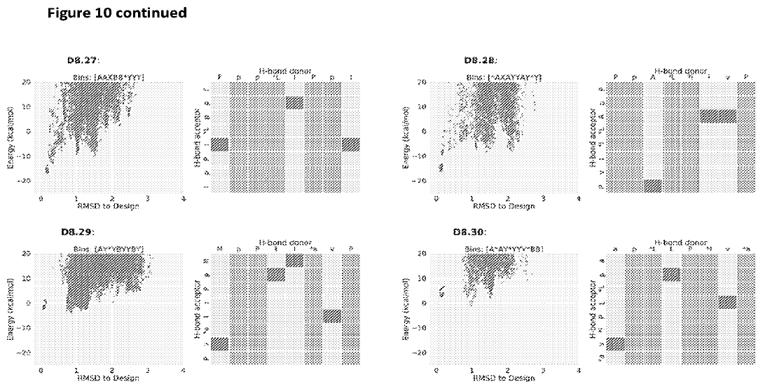

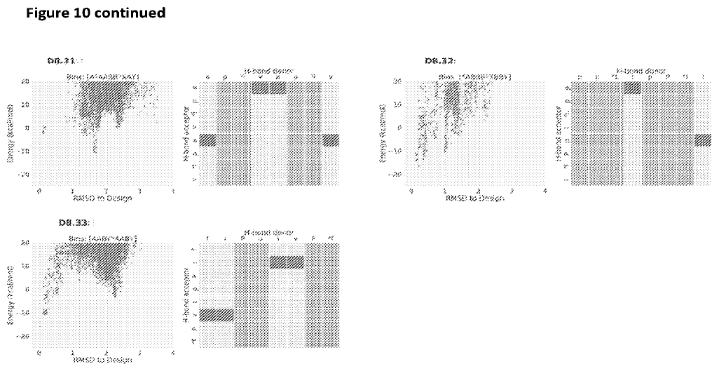

We first tested the ability of our design pipeline to control both macrocycle structure and membrane permeability on six to eight residue macrocycles spanning a diversity of structures and N-methylation patterns. For 6, 7, and 8 residue macrocycles we selected 8, 5, and 19 designs (representing 6, 5, and 16 clusters) respectively with fully satisfied backbone NH groups and funnel-like energy landscapes and spanning a diversity of sequences, structures, N-methylation patterns, and structural motifs (FIGS. 8-10 and Table 2). Selected macrocycles were chemically synthesized and purified using reverse-phase high-performance liquid chromatography (HPLC). Isobaric peaks matching the expected mass during the purification were collected and experimentally tested separately (labeled as p1 and p2 in design names) (see Supplementary Methods).

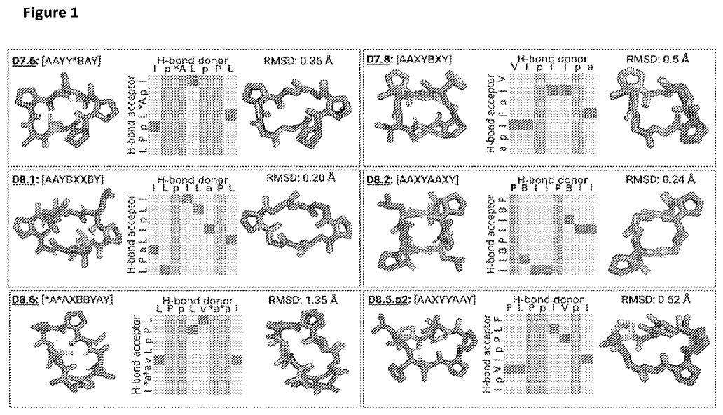

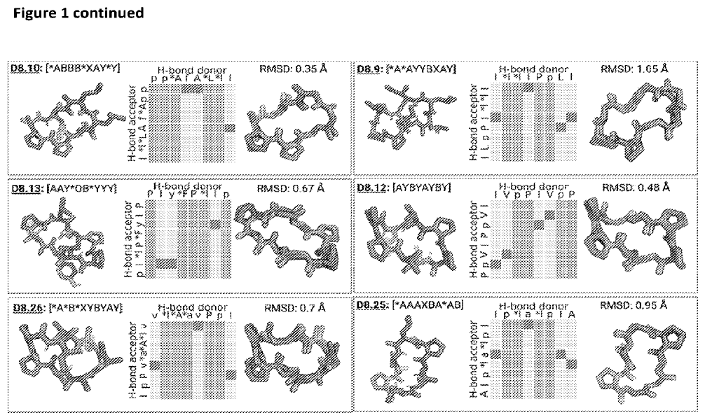

To evaluate the accuracy of the design method, we determined x-ray crystal structures for two 7-residue designs and fourteen 8-residue designs. The structures for two 7-residue and eleven 8-residue macrocycles were very close to the computational design models (backbone atom RMSD <1.2 Å) (FIG. 1 and Table 4). In six cases with remarkably low RMSDs below 0.5 Å, the design models are within the experimental resolution of the X-ray data. Three of the designs D8.1, D8.2, D8.12, are internally symmetric: D8.1 with a backbone RMSD to the design model of 0.2 Å has an internal S2 mirror symmetry composed of four beta turns and two gamma turn motifs, D8.2 has C2 symmetry with a combination of 4 beta turns and 2 alpha turns (RMSD 0.24 Å), and D8.12 has C2 symmetry with four internal hydrogen bonds and no N-methylated amino acids (RMSD 0.48 Å) (FIG. 1). Design D8.5.p2 with no N-methylated amino acids is stabilized by four internal hydrogen bonds forming four beta turns and an alpha turn motif (RMSD 0.52 Å). D8.10 with 0.35 Å RMSD between model and X-ray structure has three N-methylated amino acids and two proline residues in its sequence; its other three amino acids are involved in three internal hydrogen bonds. D7.6 features three beta turns and single N-methylated amino acid, and matches very closely with the design (RMSD 0.35 Å). D7.8 has five internal hydrogen bonds and no N-methylated amino acids (RMSD 0.5 Å). Overall, the close matches between the experimental structures and the design models show that our approach can very accurately specify macrocycle structure (Table 3).

TABLE 3
RMSD between the designed models and X-ray crystal structures of the 7- and
8-amino acid macrocycles,

| | | RMSD backbone | |
|---|---|---|---|
| | RMSD (CA) | (C + N + CA + O + CN) | RMSD All atoms |
| Design Name | (Å) | (Å) | (Å) |
| D7.6 | 0.22 | 0.35 | 1.51 |
| D7.8 | 0.31 | 0.5 | 1.44 |
| D8.1 | 0.2 | 0.2 | 0.78 |
| D8.2 | 0.2 | 0.24 | 0.86 |
| D8.3.p1 | 0.56 | 0.57 | 1.62 |
| D8.5.p2 | 0.44 | 0.52 | 0.8 |
| D8.6 | 1.04 | 1.35 | 2.04 |
| D8.9 | 0.66 | 1.05 | 1.47 |
| D8.10 | 0.22 | 0.35 | 0.96 |
| D8.12 | 0.29 | 0.48 | 0.79 |
| D8.13 | 0.63 | 0.67 | 2.16 |
| D8.14 | 0.46 | 0.5 | 1.75 |
| D8.15 | 0.34 | 0.42 | 0.98 |
| D8.17 | 1.25 | 1.7 | 3.52 |
| D8.19 | 2.43 | 2.24 | 5 |
| D8.25 | 0.28 | 0.95 | 1.56 |
| D8.26 | 0.57 | 0.7 | 1.07 |

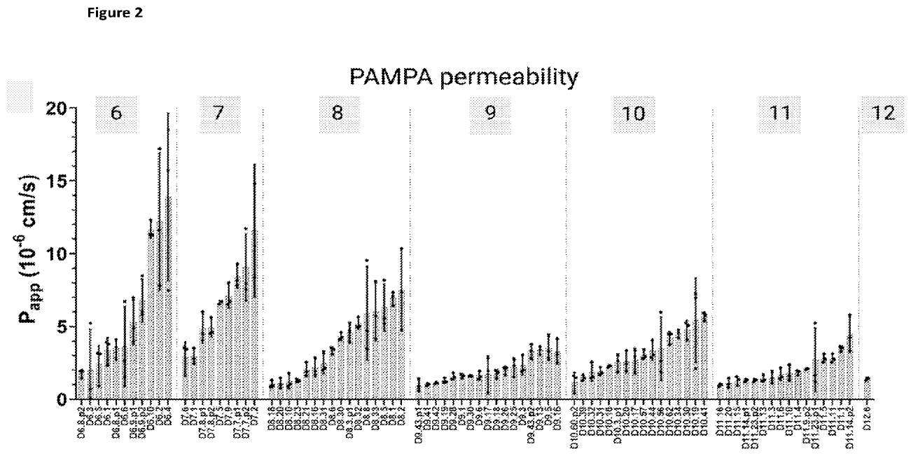

Having found that the macrocycles fold as designed, we next investigated their membrane permeabilities using transwell permeability assays. The rates of traversal across artificial membranes in parallel artificial membrane permeability assays (PAMPA) 18 were determined by mass spectrometry-based quantification of peptide concentrations in the donor and acceptor wells (see Supplementary Methods). Eight 6-mers, five 7-mers, and fourteen 8-mers had apparent permeabilities (Pₐₚₚ) greater than 1×10⁻⁷ cm/s (see Table 2). Of these, eight 6-mers, five 7-mers, and eight 8-mers had Pₐₚₚ greater than 1×10⁻⁶ cm/s (FIG. 2A).

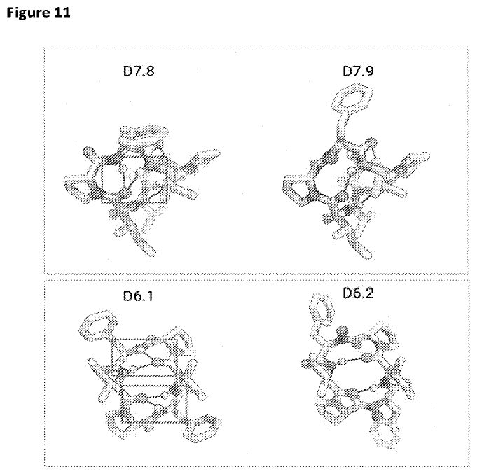

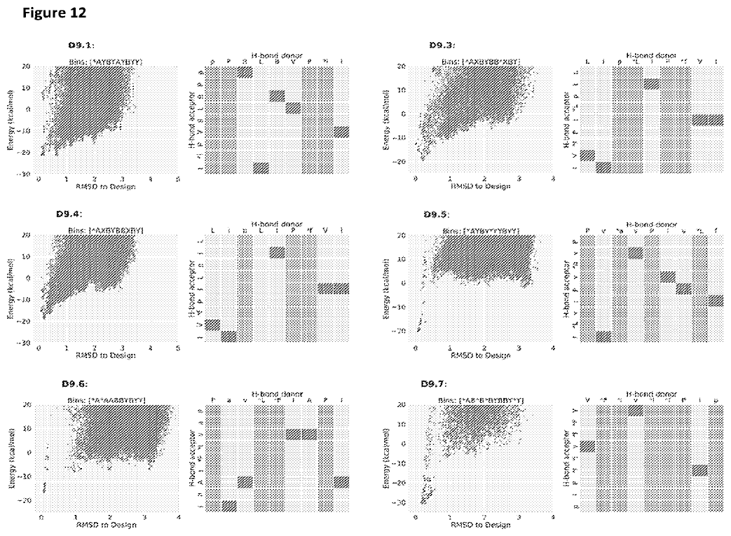

The membrane traversing macrocycles with Pₐₚₚ greater than 1×10⁻⁶ cm/s and funnel-like energy landscapes cover a wide range of structures, populating six, six, and fourteen clusters for 6, 7, and 8 residue macrocycles, respectively. For example, 7 out of 11 amino acids in cyclosporine are N-methylated, which comes at the cost of increased molecular flexibility and synthetic difficulties. In our 6-8 residue permeable designs, the number of N-methylated amino acids ranges between 0 and 3. Five of the structurally validated designs, D7.8, D8.1, D8.2, D8.5, D8.12, have no N-methyl amino acids highlighting the precise control offered by computational methods to design structures with complete hydrogen bond donor satisfaction through internal hydrogen bonding and prolines (FIG. 1 and Table 2). D8.1, D8.2, D8.5.p2, and D8.12 are the largest passively permeable macrocycles we are aware of that lack N-methylated or N-alkylated amino acids. The extensive internal hydrogen bonding enables full NH bond satisfaction in these designs with no N-methylated amino acids; designs D7.8 and D8.2 have five and six internal hydrogen bonds, respectively, that stabilize the structure. Some of the 6 and 7 residue design models (D6.3, D6.5, D6.9, and D7.8) contain a geometrically strained arrangement of overlapping beta and gamma turns (FIG. 11), in which the middle amino acid may have a partially or fully unsatisfied NH group. Accordingly, we also designed and tested variants with the middle amino acid N-methylated, and in all cases, the variant with the additional N-methylation is more permeable than the original. However, across all the permeable designs, permeability does not correlate with the number of N-methylated amino acids; for example, D8.1 is the most permeable 8 residue design in PAMPA and does not have any N-methylated amino acids. This illustrates that maximizing hydrogen bond satisfaction and using N-methylation sparingly in the folded structure is a viable strategy for achieving permeability.

### Design of Membrane-Permeable 9-12 Residue Macrocycles

Earlier work on passively permeable peptides has been primarily limited to 5-7mers because purely lipophilic peptides show a steep decrease in permeability with size. To determine whether our design principles can circumvent this trend, we used our computational pipeline to design a wide variety of larger macrocycles ranging from 9 to 12 amino acids.

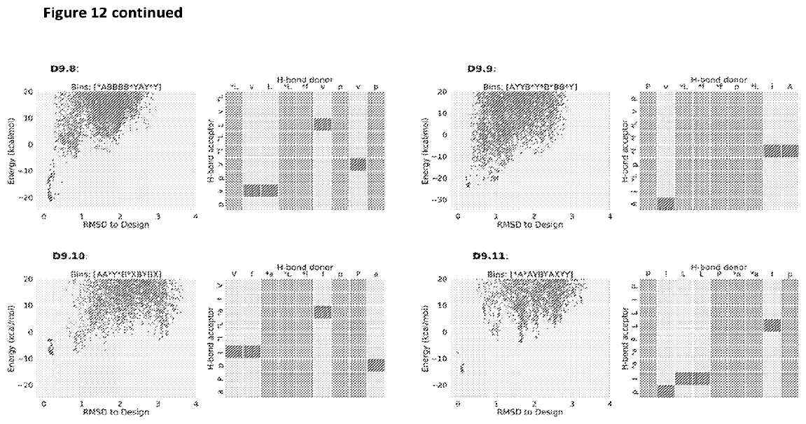

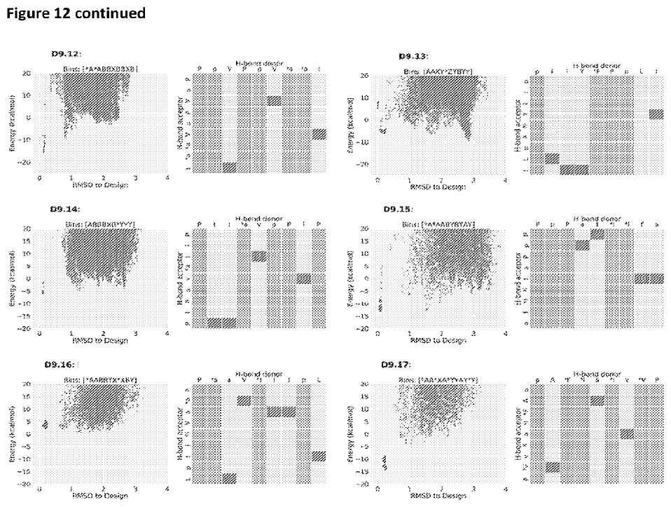

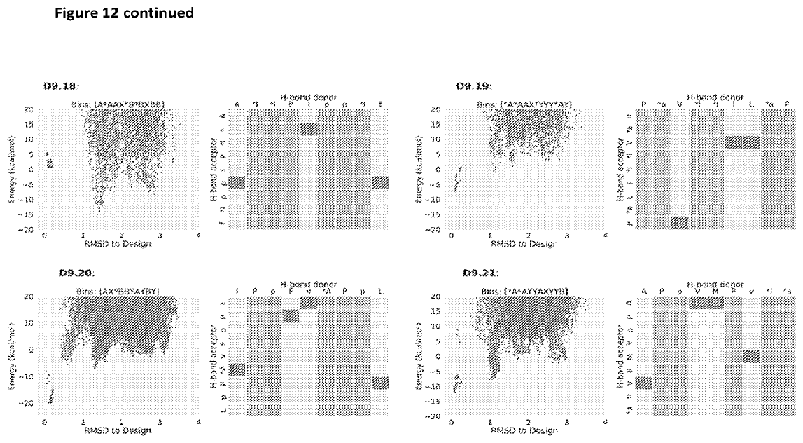

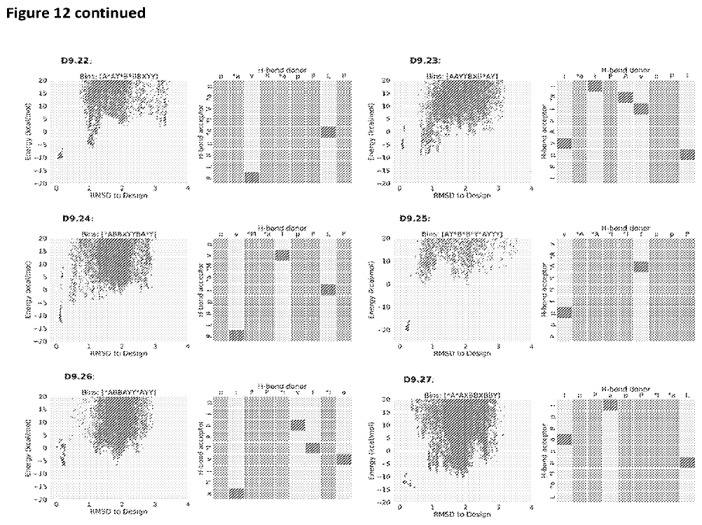

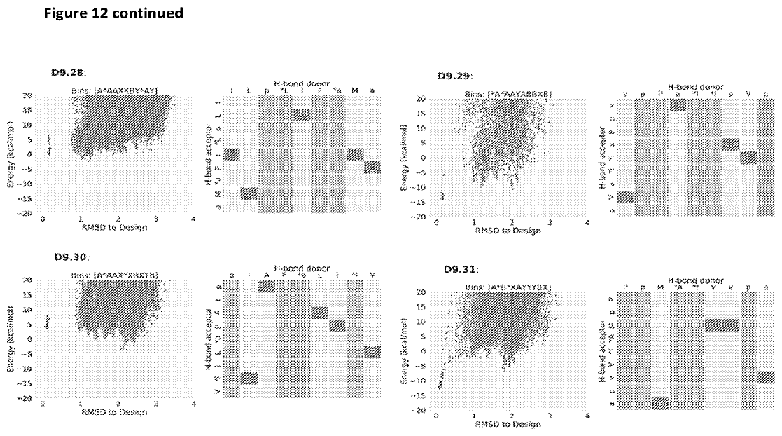

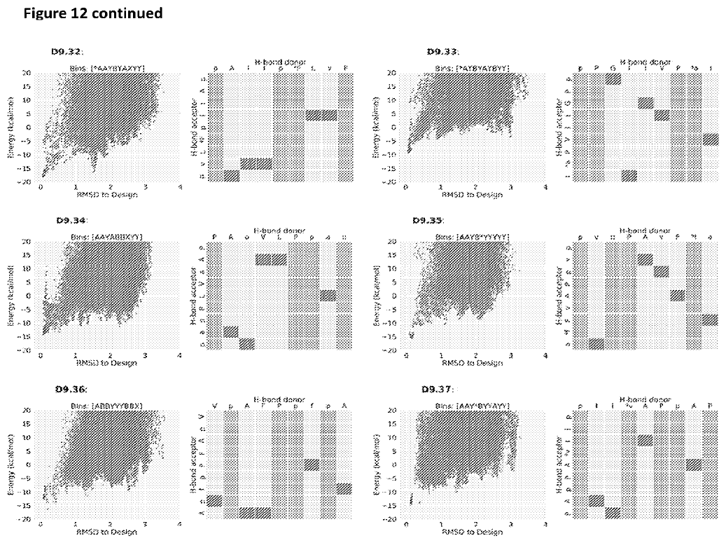

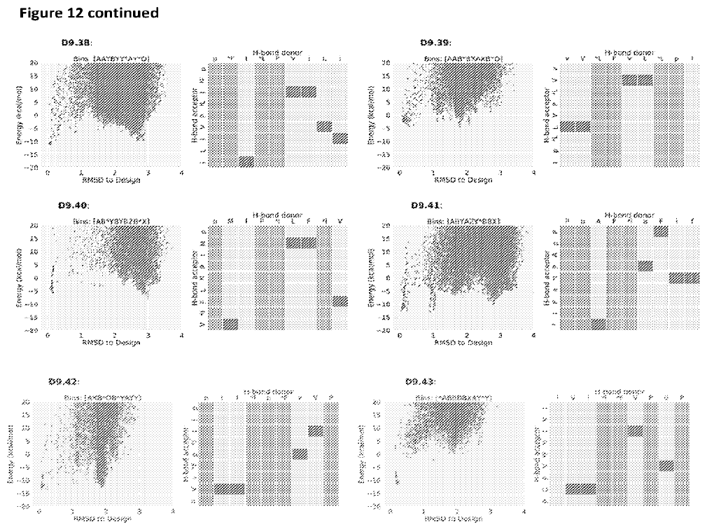

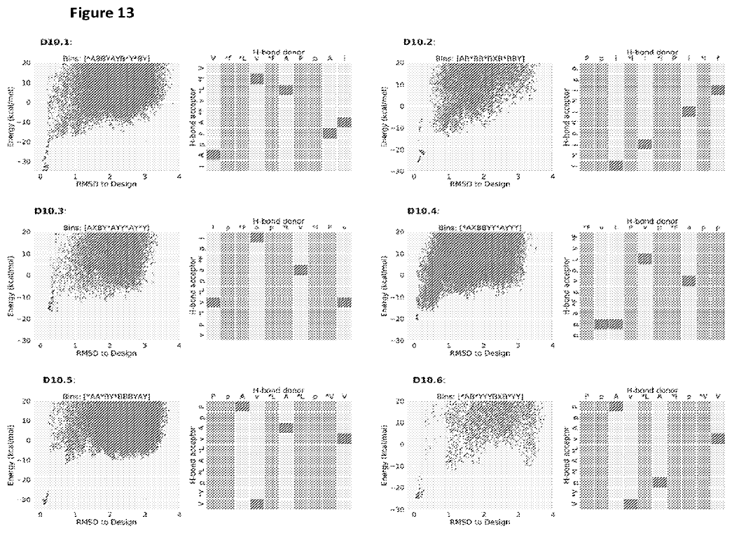

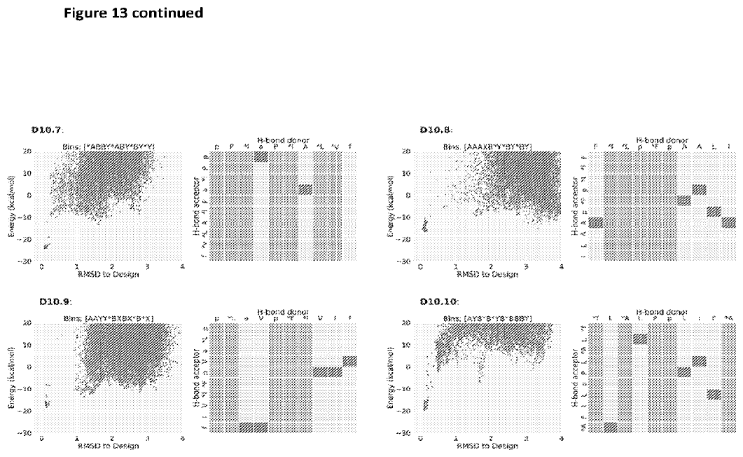

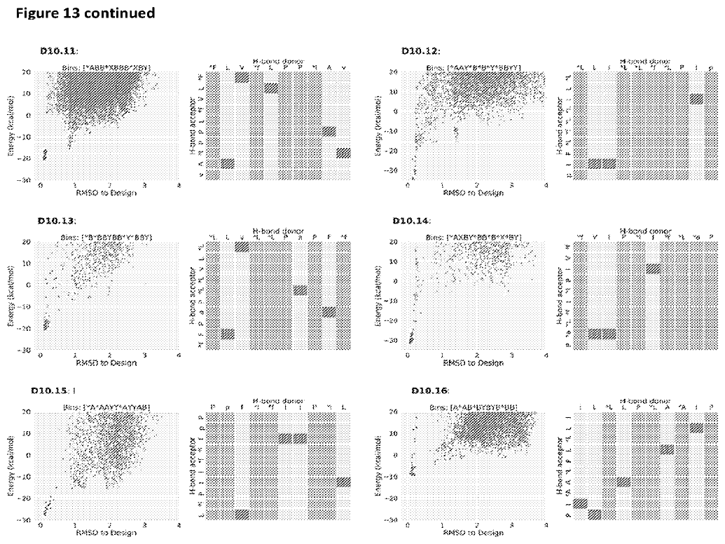

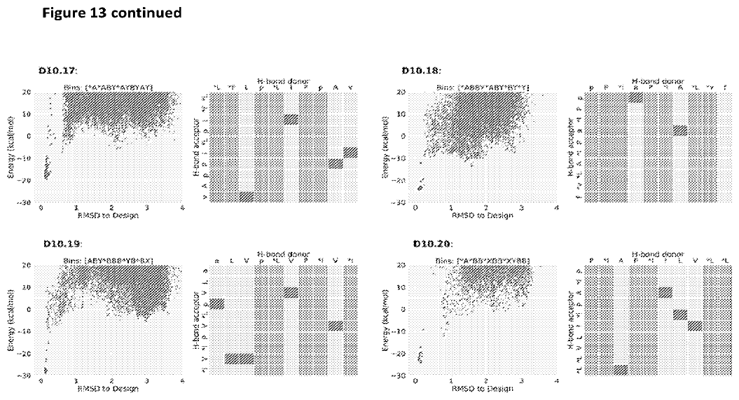

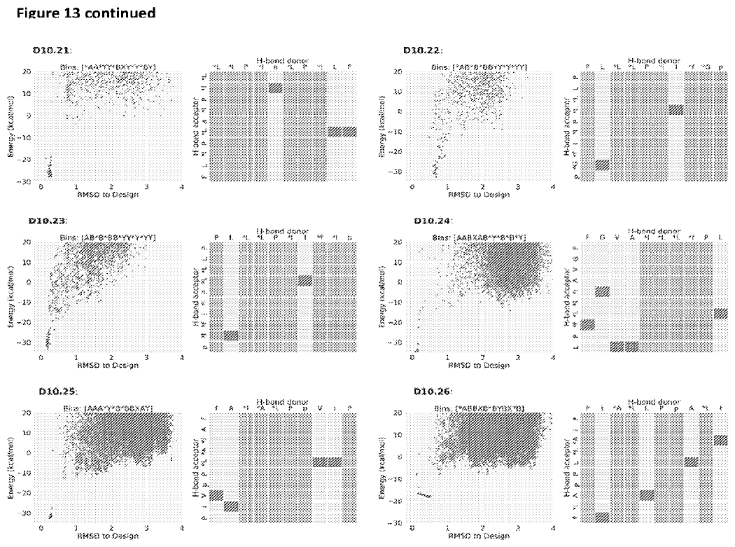

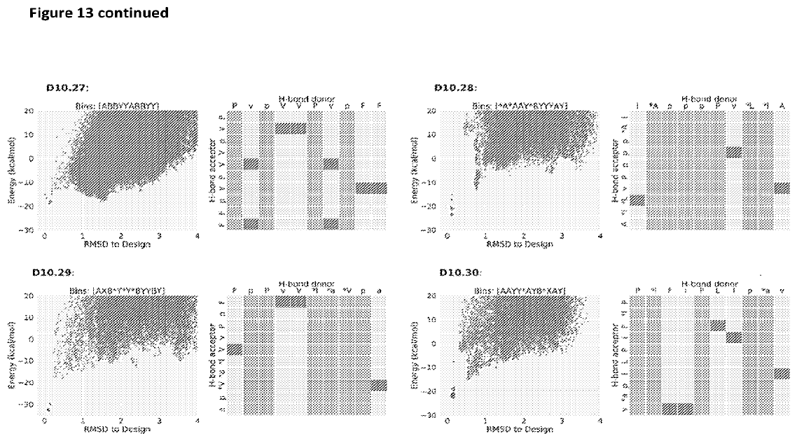

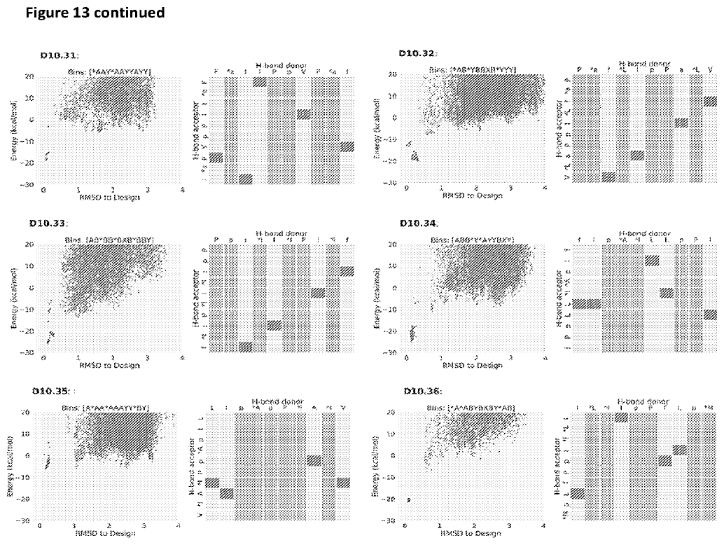

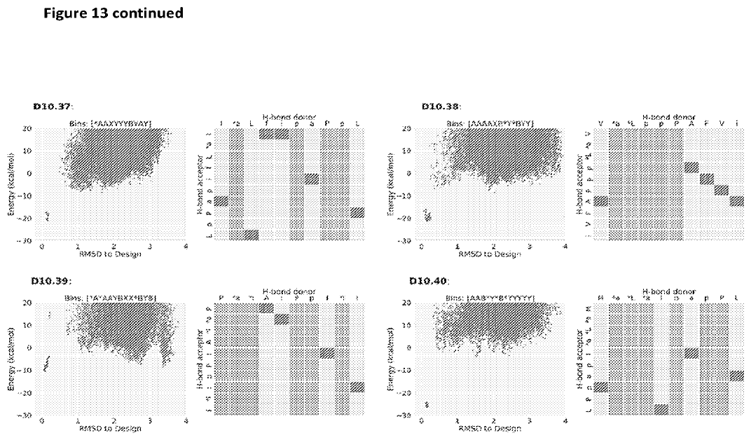

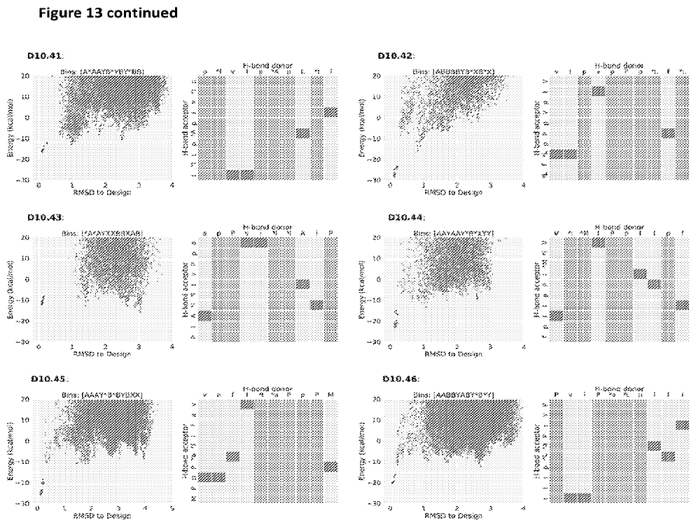

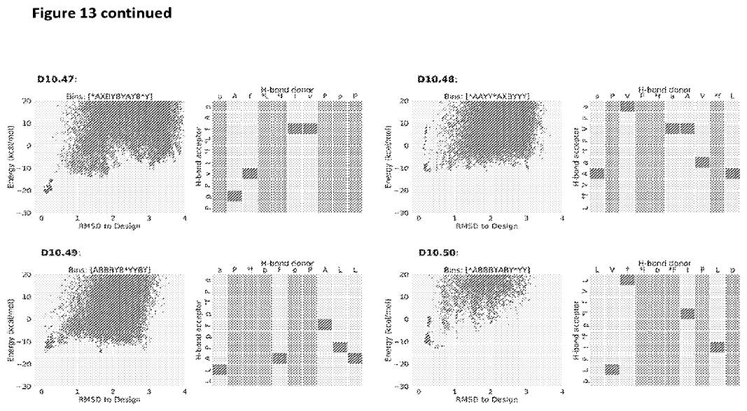

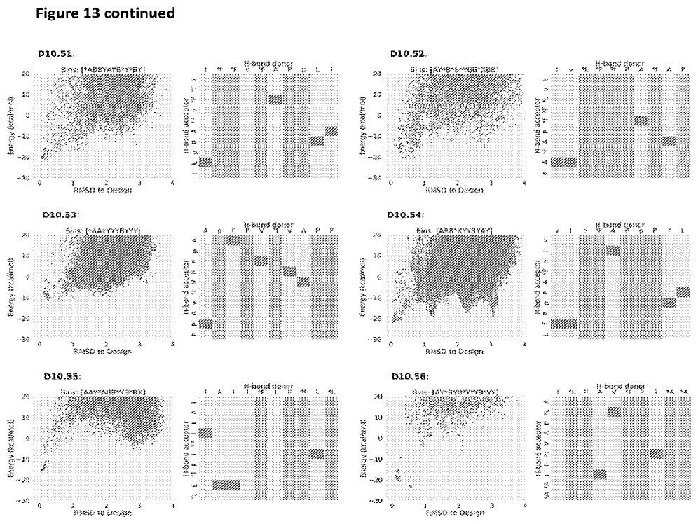

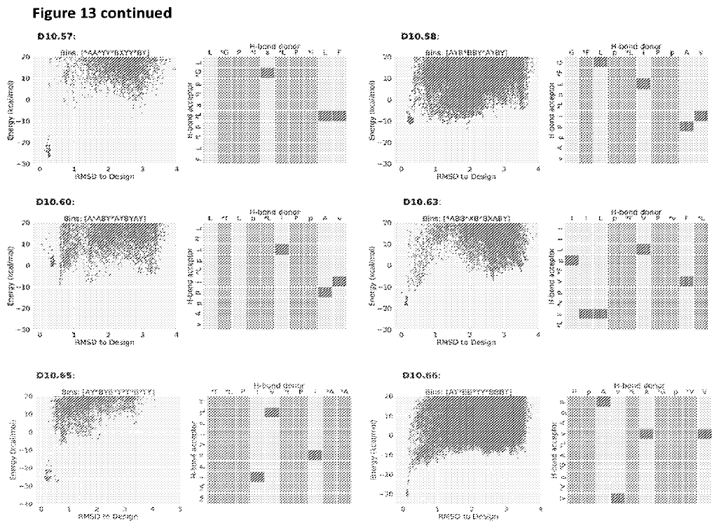

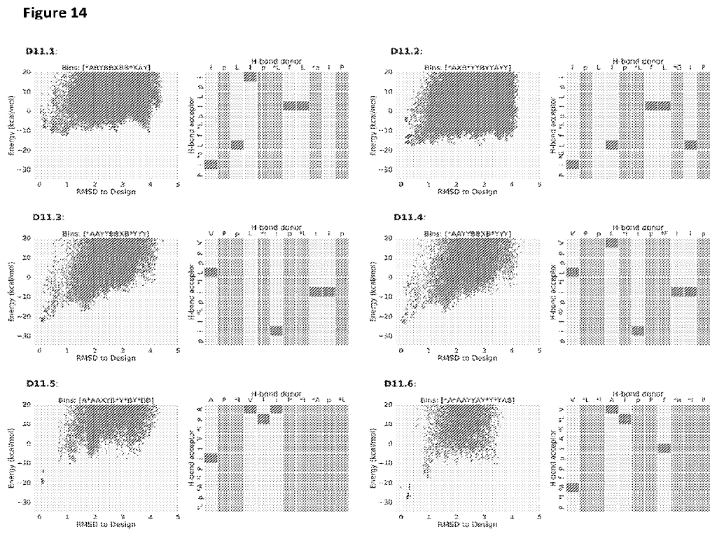

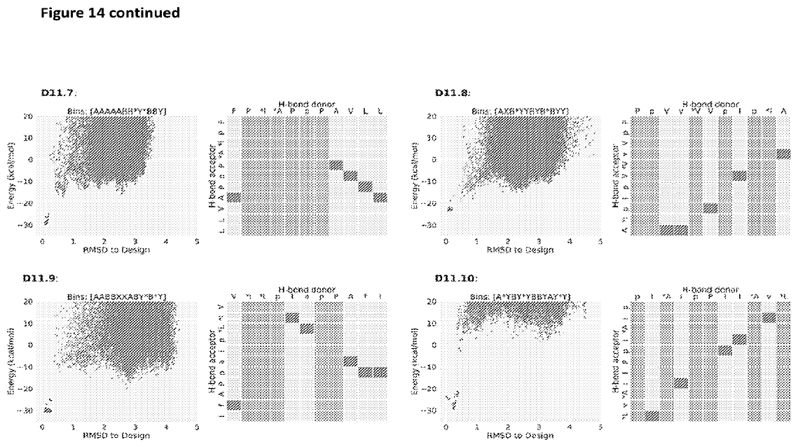

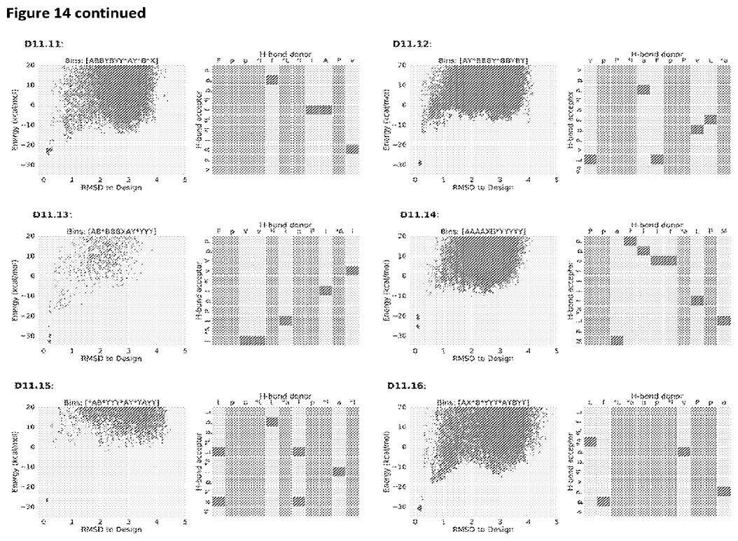

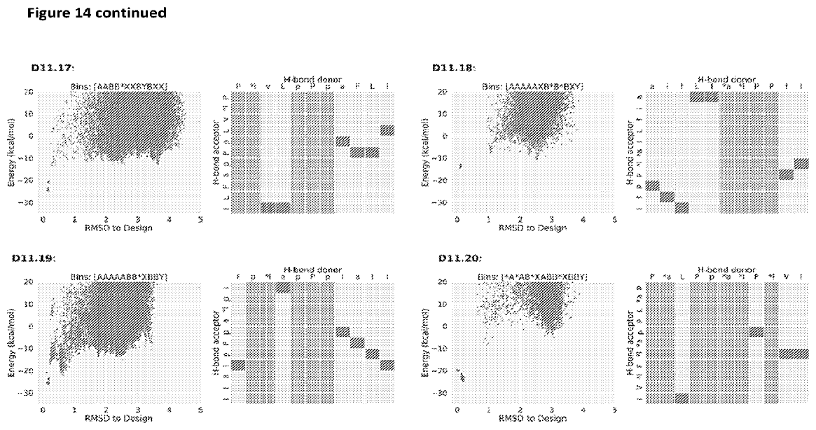

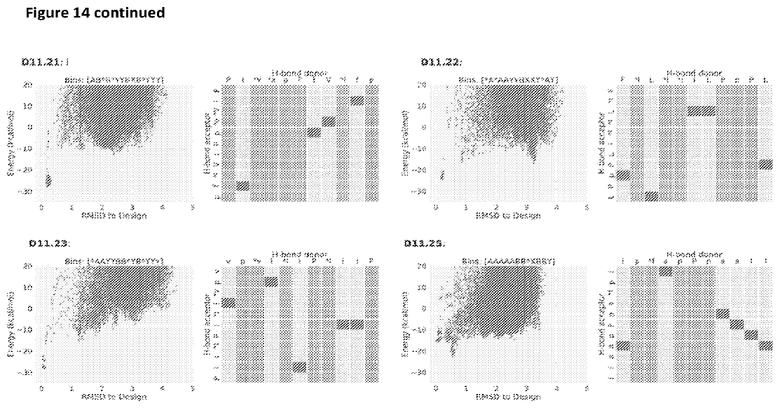

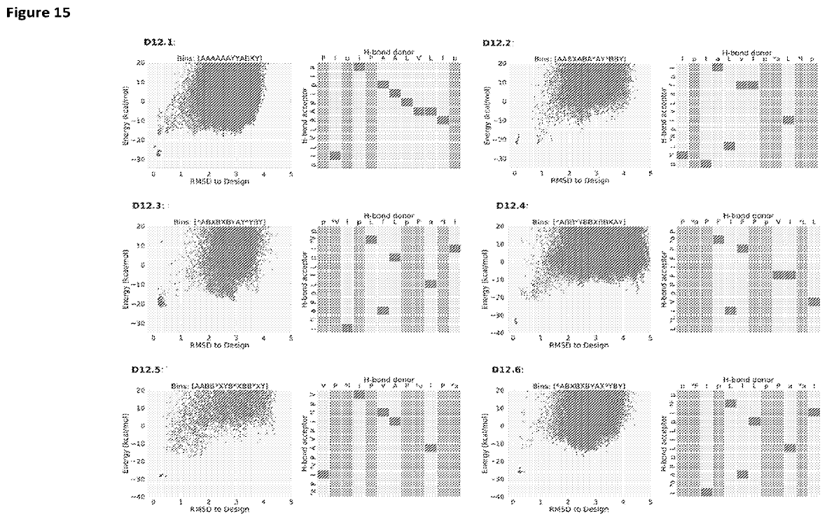

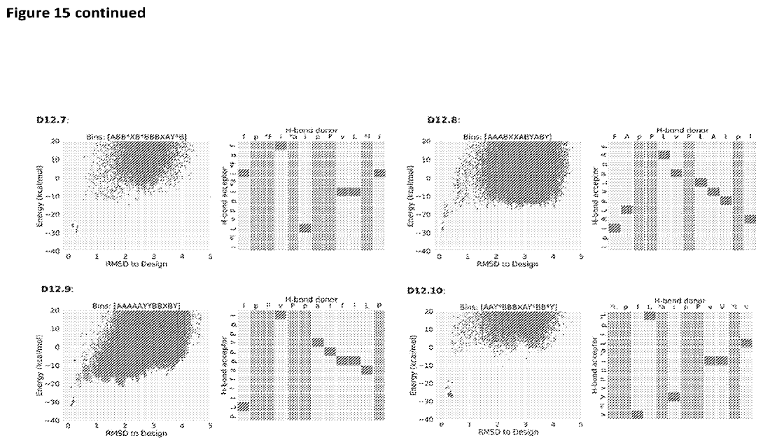

We selected for synthesis and characterization seventeen 9-mer, forty-three 10-mer, nineteen 11-mer, and eight 12-mer macrocycles with funnel-like energy landscapes spanning 16, 38, 18, and 8 different structural clusters, respectively, with widely ranging structures and between 1 and 5 N-methylated amino acids (FIG. 12-15). 48 designs spanning 11, 22, 13, and 1 structural clusters for 9, 10, 11, and 12 residue macrocycles respectively were permeable in PAMPA assays with apparent permeability greater than 1×10⁻⁷ cm/s (see Table 2). Out of these, nine 9-mers, thirteen 10-mers, twelve 11-mers, and one 12-mer showed significant permeability (Pₐₚₚ>1×10⁻⁶ cm/s) in PAMPA assays (FIG. 2A). There was a size dependence in their permeability, but the drop-off is less steep than what is observed in previous studies on non-designed macrocycles, resulting in significant permeabilities beyond the rule-of-five space.

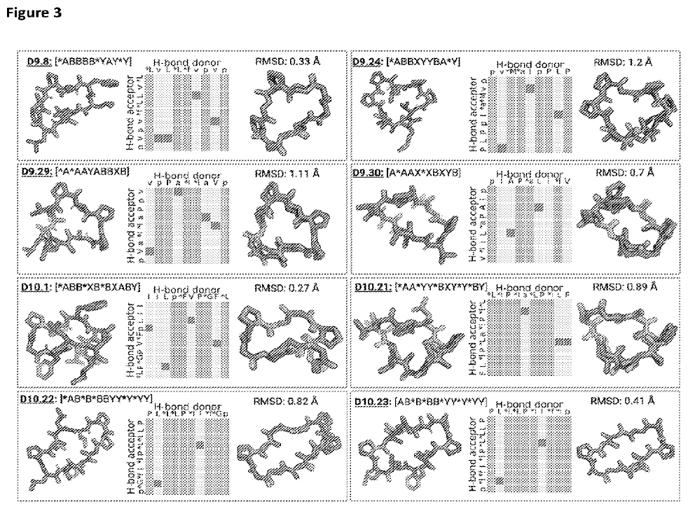

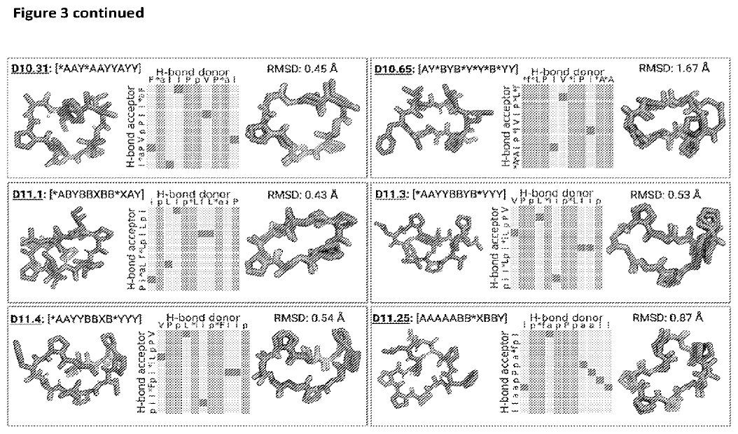

To evaluate the structural accuracy of our design models, and to confirm structure-activity relationships present in the design models and membrane permeability data described above, we sought to determine their experimental structures. We successfully crystallized and solved the high-resolution x-ray crystal structures for five 9-mers, six 10-mers, and four 11-mers. Out of these 15 structures, three 9-mers, five 10-mers, and four 11-mer macrocycles matched closely (backbone RMSD <1.2 Å) with their design models (FIG. 3 and Table 4). Design D9.8 (RMSD 0.33 Å) features three N-methylated amino acids and the structure is stabilized by 1 alpha turn, 2 beta turns, and 1 gamma turn. D10.31 (RMSD 0.45 Å) has two N-methylated amino acids and five internal hydrogen bonds stabilizing the macrocyclic structure; structures solved in isopropanol: water and ethyl acetate: pentane mixtures were identical. Design D10.1 contains five intramolecular hydrogen bonds, and the crystal structure is nearly identical to the design model (backbone RMSD of 0.27 Å and all-atom RMSD of 0.47 Å). Designs D10.21, D10.22, and D10.23 each contain five N-methylated amino acids; D10.21 is stabilized by three internal hydrogen bonds and two prolines, while D10.22 and D10.23 have two internal hydrogen bonds (backbone RMSDs of 0.89 Å, 0.82 Å and 0.41 Å, respectively). D11.3 and D11.4 have five internal hydrogen bonds, and their crystal structures have backbone RMSD less than 0.55 Å to the design model. Design D11.1, with a backbone RMSD of 0.43 Å between the design model and the X-ray crystal structure, contains five internal hydrogen bonds, 2 N-methylated amino acids, and three prolines. The structure is stabilized by three beta turns, 1 alpha turn, and one gamma turn.

TABLE 4
RMSD between the designed models and X-ray crystal structures
of the 9-11 residue macrocycles.

| | | RMSD backbone | RMSD All |
|---|---|---|---|
| | RMSD (CA) | (C + N + CA + | atoms |
| Design Name | (Å) | O + CN) (Å) | (Å) |
| D9.1 | 2.36 | 2 | 3.03 |
| D9.8 | 0.39 | 0.33 | 1.31 |
| D9.24 | 0.9 | 1.2 | 2.4 |
| D9.29 | 0.78 | 1.11 | 1.66 |
| D9.30 | 2.54 | 2.49 | 4.9 |
| D10.1 | 0.27 | 0.27 | 0.47 |
| D10.21 | 0.38 | 0.89 | 1.19 |
| D10.22 | 0.75 | 0.82 | 1.58 |
| D10.23 | 0.38 | 0.41 | 1.28 |
| D10.31 | 0.47 | 0.45 | 1.24 |
| D10.65 | 1.74 | 1.67 | 3 |
| D11.1 | 0.43 | 0.43 | 1.11 |
| D11.3 | 0.52 | 0.53 | 1.47 |
| D11.4 | 0.51 | 0.54 | 0.93 |
| D11.25 | 0.63 | 0.87 | 1.99 |

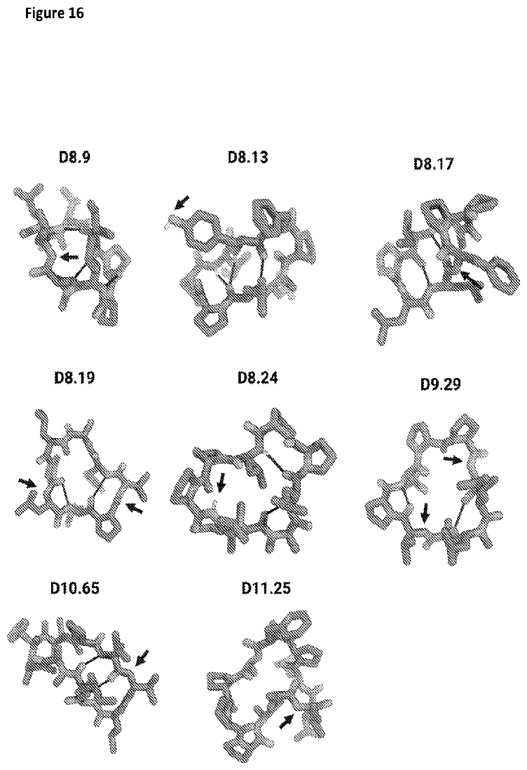

The structural accuracy of our designs coupled with a large number of permeability measurements provides insight into the relationship between permeability and NH satisfaction in these larger peptides. Overall, almost all peptides without exposed NH groups were membrane permeable. In contrast to results with non-designed macrocycles, there was not a strong correlation between permeability and size; indeed some of the 10 and 11 residue peptides were highly permeable. Crystal structures of macrocycles that were not permeable (FIG. 16) further support the importance of NH satisfaction: these did not match the (fully satisfied) design models because of disruption of the intramolecular hydrogen bonds by the insertion of a water molecule, which leads to exposure of polar groups.

### Permeability of Cis-Peptide Bond Containing Macrocycles

Our results thus far establish that computational design for satisfying all NHs can robustly generate highly membrane-permeable designs well beyond the Ro5 limits. We next investigated other possible contributions to membrane permeability. Cis peptide bonds are present in a number of our designs. Design D8.31 has cis peptide bonds at the two N-methylated D-leucines at residue positions 3 and 8 that are part of a rare beta-turn formed by a Nme-D-Pro(i+1)-Nme-D-aa(i+2) motif. Design D8.13 contains three prolines with one designed to be in a cis peptide bond stabilized by an aromatic AA(i+1)-Proline(i+2) motif recapitulated in the crystal structure. D8.6 contains two N-methylated amino acids in a row and four intramolecular hydrogen bonds; one of the N-methylated D-alanine undergoes a trans-to-cis switch in the X-ray crystal structure, but because the switch happens around an N-methylated amino acid, the overall NH satisfaction in the macrocycle is still maintained. Similarly, in design D8.9, an N-methylated D-leucine at amino acid position 3 undergoes a trans-to-cis switch but maintains the overall satisfaction of the peptide backbone, and the crystal structure of D9.16 also contains a trans-to-cis omega flip relative to the design model.

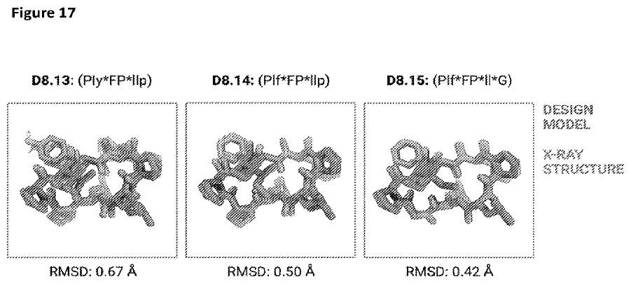

Over our macrocycle set, there is little association between the presence of cis peptide bonds and the extent of membrane permeability. The permeabilities of cis-peptide containing D8.9, D8.13, D8.14, D8.15, D8.6, D9.13, and D10.62 are in the same range as those of all-trans macrocycles with the same number of residues. The secondary contribution of cis peptide bonds is further illustrated by D8.13, D8.14, and D8.15 which have similar sequences and structures (FIG. 17) with cis peptide bonds at the same position; D8.13 is not membrane permeable, and D8.14 and D8.15 have PAMPA Pₐₚₚ of 3.68×10⁻⁷ cm/s and 7.68×10⁻⁷ cm/s. D8.13 has a tyrosine residue in place of a phenylalanine residue, creating an unsatisfied OH group that likely prevents permeability.

### Design of Membrane-Permeable Chameleonic Macrocycles

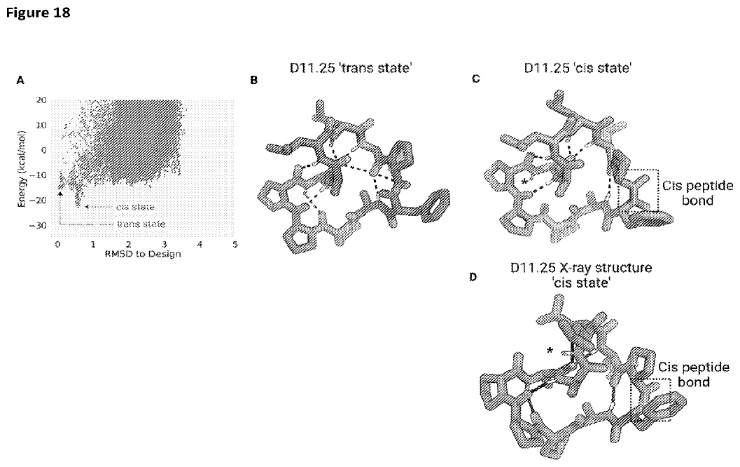

While the above results suggest that cis peptide bonds do not inherently increase membrane permeability, we reasoned that cis-trans isomerization of the peptide bond could be a powerful design principle for generating peptides with both an open state, with polar groups exposed to interact with a therapeutic target, and a membrane-permeable closed state, with all NH groups making intra-peptide hydrogen bonds. The relatively slow (seconds timescale) kinetics of cis-trans isomerization allows for a peptide to populate multiple conformations that can interconvert fast enough to be biologically relevant, but are distinguishable by NMR. To test this hypothesis, we designed macrocycles that undergo cis-trans isomerization. D11.25 provides a start in this direction: the structure prediction calculations identified two very similar conformations that differ by a trans-to-cis peptide bond flip around the only N-methylated amino acid in the sequence (FIG. 18). The all-trans conformation is stabilized by six intramolecular hydrogen bonds that satisfy all the available NH groups, while the cis form, which is closely recapitulated in the crystal structure (backbone RMSD 0.53 Å), exposes one NH group from D-leucine at position 10 (FIG. 3 and Supplementary FIG. 18).

We set out to systematically design chameleonic peptides with two distinct energy minima differing by isomerization around a peptide bond: one which has no exposed amides and can traverse membranes, and one which exposes amides to solution and hence has the potential to bind polar target sites. The key design strategy is to find peptides that can populate multiple distinct but almost isoenergetic states (with differences in Rosetta™ calculated energies of less than 5 kcal/mol). We used three approaches to identify such peptides. First, we used large-scale structure prediction calculations to generate energy landscapes for many designs (FIGS. 8-10 and 12-15), searched for those with two or more minima, and identified 46 with Rosetta™ calculated energy differences between the two states of less than 5 kcal/mol. Second, for 20 additional cases, where the calculated energy difference between the states was >5 kcal/mol, we developed a genetic algorithm-based multi-state design method (see Supplementary Methods) to optimize the sequence such that the two alternative states have similar energies. Full energy landscapes calculations were then performed for these new sequences to confirm the presence of two isoenergetic minima. Third, starting from crystallographically confirmed designs, we introduced destabilizing mutations that led to a second low-energy minimum in the energy landscape, and generated an additional 3 peptides predicted to adopt two states.

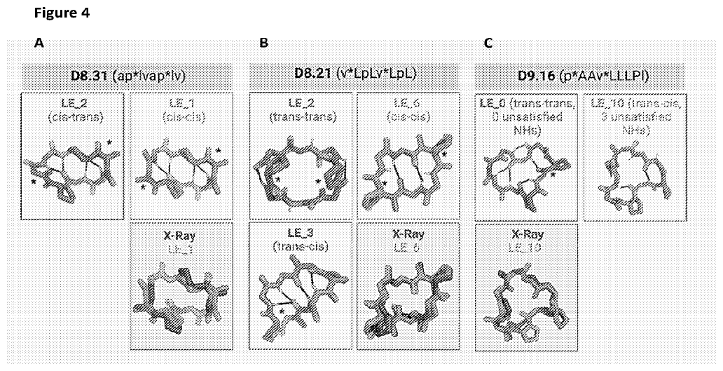

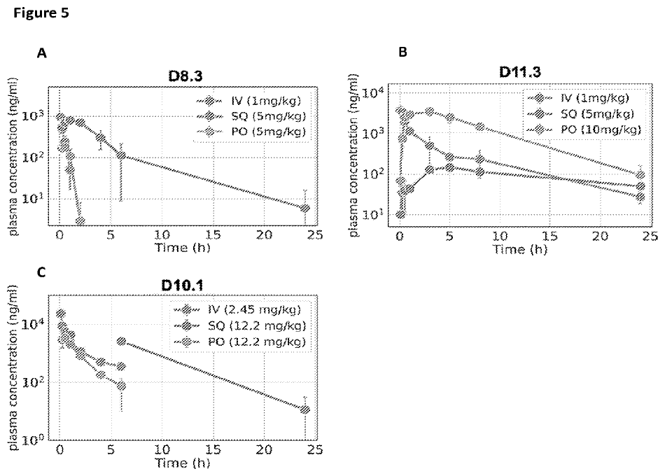

We synthesized 69 macrocycles spanning two 6-mers, two 7-mers, fourteen 8-mers, twenty-five 9-mers, nineteen 10-mers, five 11-mers, and two 12-mers predicted to have alternative low energy states. Out of these, 49 macrocycles have PAMPA Pₐₚₚ greater than 1×10⁻⁷ cm/s, and 22 show significant apparent permeability (Pₐₚₚ>1×10⁻⁶ cm/s) (FIG. 2A and Table 2). We selected 19 peptides that showed significant permeability for further studies with NMR. The lower success rate in these designs is consistent with their greater polarity, the challenges in designing multiple states, and possibly much slower membrane transversal rates limited by the kinetics of conformational isomerization. ID 1H NMR in de-DMSO and CDCl₃ indicated that seven of these designs have a strong solvent dependence of the equilibrium between the structured conformations, switching conformational states, or ratios of conformational states, between polar and non-polar solvents (see Table 2). Energy landscape calculations also revealed that switch design generally resulted in more than two discrete low energy states—an expected consequence of introducing interactions that favor more than one state. We clustered the low energy ensembles from structure prediction calculations for each macrocycle and assigned each distinct minimum a state (cluster) identifier ranked on the lowest energy structure in that cluster. For three of these seven macrocycles (D8.21, D8.31, and D9.16) designed with the first approach, we succeeded in solving crystal structures matching the design model or one of the predicted alternate low-energy structures (FIG. 4).

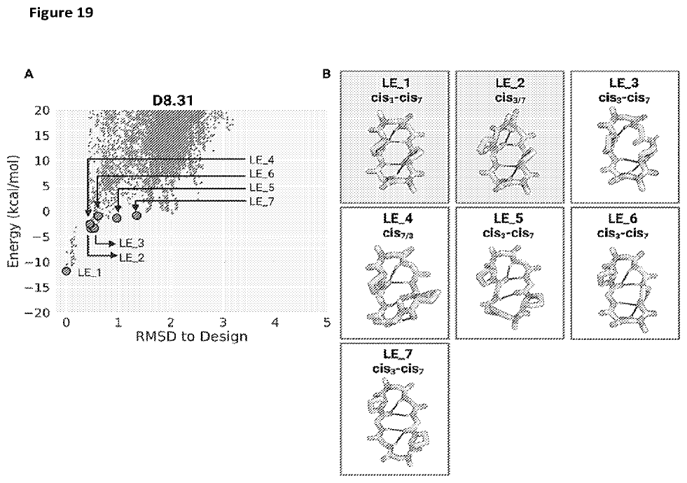

Peptide D8.31 is an 8 amino-acid macrocycle with a symmetric repeat sequence (ap*lvap*lv (SEQ ID NO:43), * represents N-methylated amino acids); the lowest energy state (LE_1) is C2 symmetric with both N-methylated amino acids in cis peptide bond conformations (“cis-cis”), and the second-lowest energy alternative state (LE_2) is asymmetric with one N-methylated leucine in the cis conformation (“cis-trans”) (FIG. 4, left panel; FIG. 19). The cis-trans isomerization occurs around an N-methylated amino acid, and hence both states have no unsatisfied NH groups. The crystal structure in the ethyl acetate: pentane solution is similar to the cis-cis LE_1. The correspondence between these experimental X-ray crystal structures and the predicted low-energy states for D8.31 demonstrates that Rosetta™ calculations can guide the design of macrocycles adopting multiple states. However, as the two different states have the same number of exposed NHs, these data do not directly address the contribution of conformational switching to membrane permeability. More relevant are the two other macrocycles, D8.21 and D9.16.

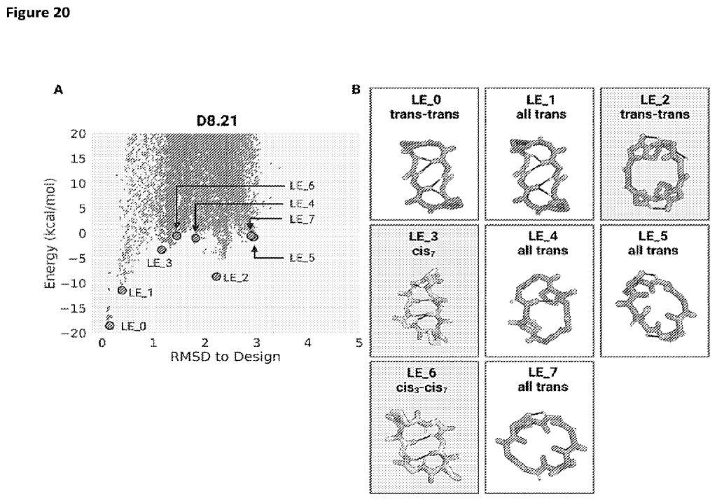

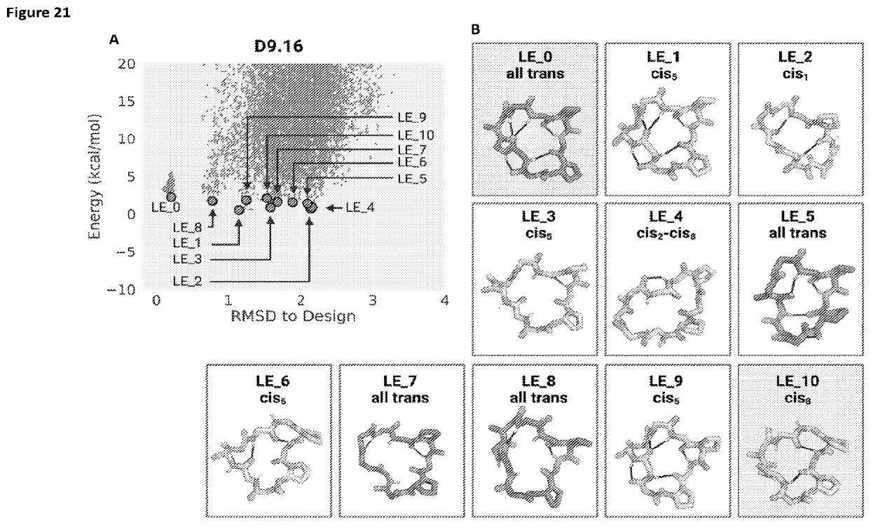

Macrocycle D8.21 also has a symmetric repeat sequence (v*LpLv*LpL; SEQ ID NO: 36) with predicted low energy “trans-trans” states (2 variants, LE_1 and LE 2), “trans-cis” states (LE_3), and “cis-cis” (LE_6) states (FIG. 20). The trans-trans LE_2 state has exposed NH groups, as well as two NHs forming surface exposed hydrogen bonds. Both the trans-cis LE_3 and cis-cis LE_6 states have saturated NH groups that form hydrogen bonds in the core of structure. The X-ray crystal structure of D8.21 in aqueous conditions is a cis-cis conformation similar to LE_6 (FIG. 4, middle panel). Design D9.16 has two N-methylated amino acids and two prolines (p*AAv*LLLPl; SEQ ID NO:61). The low energy design model is a “trans-trans” conformation (LE_0) with no unsatisfied NH groups. The predicted low-energy states include a “trans-cis” state (LE_10) with exposed NH groups (FIG. 4, right panel, and FIG. 19). The X-ray crystal structure from aqueous conditions is in a trans-cis conformation that matches LE 10.

Taken together, these data indicate that the D8.21, D8.31, and D9.16 macrocycles indeed populate multiple states, with the low-energy states closely matching the experimental crystal structures. The macrocycles are also, as intended by design, membrane permeable, but due to difficulties in characterizing the state of macrocycles during membrane traversal, we cannot attribute this permeability specifically to one of the designed states. Boding well for the future design of membrane-permeable macrocycles targeting polar binding sites, it is notable that both D8.21 and D9.16 expose backbone NHs in one state, yet retain significant permeability.

### Oral Bioavailability

Oral bioavailability is a desirable therapeutic property that requires stability against the low pH and proteases in the gastrointestinal tract and permeation across the epithelial cells in the gut. We tested the plasma exposure in mice or rats for a subset of the designs with good permeability—one 8-mer (D8.3.p1), one 10-mer (D10.1), and one 11-mer peptide (D11.3)—after single-dose administration via intravenous (IV), subcutaneous (SQ), and oral (PO) routes. The amount of unmodified drug in plasma was quantified by mass spectrometry and the fraction of unmodified drug (% F) in plasma after oral delivery was determined using IV dosing as reference (see Supplementary Methods). All of the peptides were well tolerated without any adverse effects at the doses tested. All four designs had substantial oral exposure and demonstrated comparable or better oral bioavailability than most other natural orally absorbed peptides. Designs D8.3.p1, D11.2, and D10.1 have a good % F between 7.5-11% (FIG. 5 and tables 5-15). The 11-mer design, D11.3, was tested for oral bioavailability in male Swiss albino mice and had a very high oral bioavailability (% F) of 40% despite its large size. The designs also demonstrated other favorable drug-like attributes, such as long plasma half-life (T_{1/2}). D11.3 has a T_{1/2} of 5.58 hours after IV dosing and D10.1 has a T_{1/2} of 3.75 hours after SQ administration (FIG. 5 and Tables 5-15). Overall, these in vivo data validate that these computationally designed and structurally validated peptides are robust to low pH and protease exposure, and get absorbed efficiently across the gut epithelial barrier.

### Conclusion

We have shown that the ability to precisely control structure enables the robust design of a wide diversity of membrane-permeable macrocycles. Our designs achieve this high permeability through conformational shielding of polar groups using a diversity of local structures and internal hydrogen bonds. In total, we designed, synthesized, and validated 82 structurally diverse macrocycles with good to excellent permeability, including 6-8 residue macrocycles with high permeability and no N-methyl groups, and 9-12 residue membrane-permeable macrocycles with a single N-methylated amino acid in the sequence. The passive permeability of the designs in PAMPA translates to good oral availability in rodent models. The importance of computational-design based control over structure is highlighted by the strong correlation between the extent of permeability and sub-angstrom match between experimental structure and design model: of the 34 designs for which we succeeded in determining crystal structures, 21 macrocycles out of the 25 that closely matched (RMSD <1 Å) the design states were all membrane permeable (Pₐₚₚ>1×10⁻⁷ cm/s). While the very close agreement (RMSD <1.2 Å) between the models of 29 out of the 35 designs and the corresponding experimental structures indicates that the design method has very high accuracy, we cannot exclude the possibility that designs for which we lack X-ray fold into alternate conformations important for permeability.

The design methods and membrane-permeable macrocycles presented herein provide access to intracellular targets and oral delivery. Our energy landscape-based approach to designing peptides that exhibit chameleonic behavior, switching between a lipophilic state and a second, relatively polar, state as solvent polarity changes provides for macrocycles that can bind intracellular therapeutic targets with exposed polar groups while retaining membrane permeability.

TABLE 5
Pharmacokinetic data for D8.3.p1: Mean plasma levels after PO, SQ, and IV
dosing in female Balb/C female mice

| | | D8.3.p1 Mean plasma levels (μM) | | | |
|---|---|---|---|---|---|
| | (min) | PO | SQ | IV | |
| | 5 | | | 1.07 ± | |
| | | | | 0.07 | |
| | 15 | 0.19 ± | 0.61 ± | 0.53 ± | |
| | | 0.04 | 0.13 | 0.11 | |
| | 30 | 0.19 ± | 0.78 ± | 0.27 ± | |
| | | 0.03 | 0.28 | 0.09 | |
| | 60 | 0.12 ± | 0.9 ± 0.05 | 0.06 ± | |
| | | 0.04 | | 0.04 | |
| | 120 | 0 ± | 0.78 ± | | |
| | | 0.01 | 0.09 | | |
| | 240 | | 0.34 ± | | |
| | | | 0.17 | | |
| | 360 | | 0.13 ± | | |
| | | | 0.12 | | |
| | 1440 | | 0.01 ± | | |
| | | | 0.00 | | |
| Time of max concentration | Tmax | 25 ± 0.01 | 50 ± 17.32 | | (min) |
| Max concentration | Cmax | 0.21 ± 0.02 | 0.95 ± | | (μM) |
| | | | 0.12 | | |
| Initial concentration | C0 | | | 1.53 ± | (μM) |
| | | | | 0.07 | |
| Volume of distribution @ | VSS | | | 0.72 ± | (L/kg) |
| steady state | | | | 0.09 | |
| Volume of distribution during | VZ | | | 0.78 ± | (L/kg) |
| elimination phase | | | | 0.17 | |
| Area-under-the-curve to last | AUC0-t | 10.93 ± | 223.75 ± | 25.44 ± | (min* |
| measureable concentration | | 4.89 | 133.45 | 3.73 | μmol/L) |
| Area-under-the-curve | AUC0-∞ | 14.30 ± | 246.92 ± | 26.62 ± | (min* |
| extrapolated to infinity | | 3.71 | 99.85 | 3.96 | μmol/L) |
| Half-life | T1/2 | 29.15 ± | 154.61 ± | 12.08 ± | (min) |
| | | 14.12 | 83.41 | 2.13 | |
| Apparent clearance | CI/F | 8.34E−03 | 5.02E−04 | | (L/min) |
| Plasma clearance | CLP | | | 41.78 ± | (mL/min/ |
| | | | | 6.42 | kg) |
| Mean residence time | MRT | | | 14.60 ± | (min) |
| | | | | 1.29 | |
| Oral bioavailability | F | 10.74 ± | | | % |
| | | 2.79 | | | |

TABLE 6
Plasma levels (μM) of D8.3.p1 after PO dosing (5 mg/kg) in female
Balb/C female mice

| (min) | MI | M2 | M3 | AVG | |
|---|---|---|---|---|---|
| 15 | 0.22 | 0.2 | 0.14 | 0.19 | |
| 30 | 0.17 | 0.23 | 0.19 | 0.19 | |
| 60 | 0.06 | 0.18 | 0.12 | 0.12 | |
| 120 | 0 | 0.01 | 0 | 0 | |
| 240 | 0 | 0 | 0 | 0 | |
| 360 | 0 | 0 | 0 | 0 | |
| 1440 | 0 | 0 | 0 | 0 | |
| Tmax | 15 | 30 | 30 | 25 | (min) |
| Cmax | 0.22 | 0.23 | 0.19 | 0.21 | (μM) |
| AUC0-t | 8.03 | 16.58 | 8.18 | 10.93 | (min* |
| | | | | | μmol/L) |
| AUC0-∞ | 10.04 | 16.85 | 16.01 | 14.3 | (min* |
| | | | | | μmol/L) |
| T1/2 | 23.33 | 18.86 | 45.25 | 29.15 | (min) |
| CI/F | 1.12E−02 | 6.71E−03 | 7.06E−03 | 8.34E−03 | (L/min) |

TABLE 7

| Plasma levels (μM) of D8.3.p1 after SQ dosing (5 mg/kg) in | | | | | |
|---|---|---|---|---|---|
| female Balb/C female mice | | | | | |
| (min) | MI | M2 | M3 | AVG | |
| 15 | 0.51 | 0.7 | ND | 0.61 | |
| 30 | 0.54 | 1.08 | 0.71 | 0.78 | |
| 60 | 0.84 | 0.92 | 0.93 | 0.9 | |
| 120 | 0.72 | 0.73 | 0.88 | 0.78 | |
| 240 | 0.23 | 0.26 | 0.53 | 0.34 | |
| 360 | 0.15 | 0 | 0.23 | 0.13 | |
| 1440 | 0 | 0 | 0.02 | 0.01 | |
| Tmax | 60 | 30 | 60 | 50 | (min) |
| Cmax | 0.84 | 1.08 | 0.93 | 0.95 | (μM) |
| AUC0-t | 159 | 157.5 | 354.75 | 223.75 | (min*μmol/L) |
| AUC0-∞ | 183.06 | 195.71 | 361.99 | 246.92 | (min*μmol/L) |
| T1/2 | 111.2 | 101.86 | 250.78 | 154.61 | (min) |
| CI/F | 6.17E−04 | 5.77E−04 | 3.12E−04 | 5.02E−04 | (L/min) |

TABLE 8
Plasma levels (μM) of D8.3.p1 after IV dosing (1 mg/kg) in
female Balb/C female mice

| (min) | M1 | M2 | M3 | AVG | |
|---|---|---|---|---|---|
| 5 | 1.11 | 0.99 | 1.11 | 1.07 | |
| 15 | 0.53 | 0.42 | 0.63 | 0.53 | |
| 30 | 0.26 | 0.19 | 0.37 | 0.27 | |
| 60 | 0.1 | 0.04 | 0.03 | 0.06 | |
| 120 | 0 | 0 | 0 | 0 | |
| 240 | 0 | 0 | 0 | 0 | |
| 360 | 0 | 0 | 0 | 0 | |
| T1/2 | 14.5 | 10.5 | 11.24 | 12.08 | (min) |
| C0 | 1.61 | 1.52 | 1.47 | 1.53 | (μM) |
| VSS | 0.79 | 0.75 | 0.62 | 0.72 | (L/kg) |
| VZ | 0.89 | 0.87 | 0.59 | 0.78 | (L/kg) |
| AUC0-t | 26.32 | 21.35 | 28.66 | 25.44 | (min*μmol/L) |
| AUC0-∞ | 28.69 | 22.06 | 29.12 | 26.62 | (min*μmol/L) |
| CLP | 37.62 | 49.17 | 38.55 | 41.78 | (mL/min/kg) |
| MRT | 15.45 | 13.12 | 15.23 | 14.6 | (min) |

TABLE 9
Pharmacokinetic data for D10.1: Mean plasma levels after PO, SQ, and IV
dosing in female Balb/C female mice

| | | D10.1 Mean plasma levels (μM) | | | |
|---|---|---|---|---|---|
| | | PO | SG | IV | |
| | 5 | | | 21.00 ± 5.99 | |
| | 15 | 2.58 ± 0.59 | 2.53 ± 1.18 | 7.58 ± 1.95 | |
| | 30 | 2.92 ± 0.63 | 3.55 ± 1.67 | 4.92 ± 2.11 | |
| | 60 | 1.78 ± 0.31 | 3.93 ± 1.78 | 2.19 ± 0.29 | |
| | 120 | 0.72 ± 0.15 | ND | 1.03 ± 0.06 | |
| | 240 | 0.16 ± 0.03 | ND | 0.44 ± 0.03 | |
| | 360 | 0.07 ± 0.06 | 2.29 ± 0.15 | 0.31 ± 0.09 | |
| | 1440 | | 0.01 ± 0.02 | | |
| Time of max | Tmax | 30 ± 0.00 | 150 ± 182.48 | | (min) |
| concentration | | | | | |
| Max concentration | Cmax | 2.92 ± 0.63 | 4.14 ± 1.51 | | (μM) |
| Initial concentration | C0 | | | 37.77 ± 21.43 | (μM) |
| Volume of | VSS | | | 0.25 ± 0.08 | (L/kg) |
| distribution @ steady | | | | | |
| state | | | | | |
| Volume of | VZ | | | 0.32 ± 0.07 | (L/kg) |
| distribution during | | | | | |
| elimination phase | | | | | |
| Area-under-the-curve | AUC0-t | 270.40 ± 55.52 | 1879.5 ± | 720.90 ± | (min*μmol/L) |
| to last measureable | | | 1501.5 | 121.67 | |
| concentration | | | | | |
| Area-under-the-curve | AUC0- | 279.94 ± 55.31 | 2867.2 ± | 751.94 ± | (min *μmol/L) |
| extrapolated to | ∞ | | 1016.5 | 110.39 | |
| infinity | | | | | |
| Half-life | T1/2 | 60.29 ± 9.35 | 225.38 ± 48.70 | 67.38 ± 6.78 | (min) |
| Apparent clearance | CI/F | 7.85E−04 | 7.96E−05 | | (L/min) |
| Plasma clearance | CLP | | | 3.26 ± 0.37 | (mL/min/kg) |
| Mean residence time | MRT | | | 59.54 ± 10.01 | (min) |
| Oral bioavailability | F | 7.48 ± 1.79 | | | % |

TABLE 10
Plasma levels (μM) of D10.1 after PO dosing (12.2 mg/kg)
in female Balb/C female mice

| (min) | M1 | M2 | M3 | AVG | |
|---|---|---|---|---|---|
| 15 | 2.22 | 3.26 | 2.26 | 2.58 | |
| 30 | 2.49 | 3.64 | 2.64 | 2.92 | |
| 60 | 1.57 | 2.14 | 1.64 | 1.78 | |
| 120 | 0.54 | 0.8 | 0.81 | 0.72 | |
| 240 | 0.13 | 0.18 | 0.17 | 0.16 | |
| 360 | 0 | 0.1 | 0.1 | 0.07 | |
| 1440 | 0 | 0 | 0 | 0 | |
| Tmax | 30 | 30 | 30 | 30 | (min) |
| Cmax | 2.49 | 3.64 | 2.64 | 2.92 | (μM) |
| AUC0-t | 216.96 | 327.78 | 266.45 | 270.4 | (min*μmol/L) |
| AUC0-∞ | 226.62 | 337.03 | 276.18 | 279.94 | (min*μmol/L) |
| T1/2 | 49.88 | 63.05 | 67.95 | 60.29 | (min) |
| CI/F | 9.44E−04 | 6.35E−04 | 7.75E−04 | 7.85E−04 | (L/min) |

TABLE 11

| Plasma levels (μM) of D10.1 after SQ dosing (12.2 mg/kg) in | | | | | |
|---|---|---|---|---|---|
| female Balb/C female mice | | | | | |
| (min) | M1 | M2 | M3 | AVG | |
| 15 | 1.39 | 2.44 | 3.75 | 2.53 | |
| 30 | 1.81 | 3.69 | 5.15 | 3.55 | |
| 60 | 1.87 | 4.88 | 5.03 | 3.93 | |
| 120 | ND | ND | ND | ND | |
| 240 | ND | ND | ND | ND | |
| 360 | 2.4 | ND | 2.19 | 2.29 | |
| 1440 | 0 | 0.03 | 0 | 0.01 | |
| Tmax | 360 | 60 | 30 | 150 | (min) |
| Cmax | 2.4 | 4.88 | 5.15 | 4.14 | (μM) |
| AUC0-t | 730.93 | 3578.6 | 1329.09 | 1879.54 | (min*μmol/L) |
| AUC0-∞ | NA | 3585.94 | 2148.41 | 2867.17 | (min*μmol/L) |
| T1/2 | NA | 190.94 | 259.81 | 225.38 | (min) |
| CI/F | NA | 5.97E−05 | 9.96E−05 | 7.96E−05 | (L/min) |

TABLE 12
Plasma levels (μM) of D10.1 after IV dosing (2.45 mg/kg)
in female Balb/C female mice

| (min) | M1 | M2 | M3 | AVG | |
|---|---|---|---|---|---|
| 5 | 15.45 | 20.19 | 27.35 | 21 | |
| 15 | 9.12 | 8.25 | 5.39 | 7.58 | |
| 30 | 4.05 | 3.37 | 7.33 | 4.92 | |
| 60 | 1.91 | 2.17 | 2.49 | 2.19 | |
| 120 | 1.1 | 1 | 1 | 1.03 | |
| 240 | 0.47 | 0.41 | 0.45 | 0.44 | |
| 360 | 0.37 | 0.36 | 0.21 | 0.31 | |
| T1/2 | 72.41 | 70.07 | 59.67 | 67.38 | (min) |
| C0 | 20.11 | 31.58 | 61.61 | 37.77 | (μM) |
| VSS | 0.32 | 0.28 | 0.16 | 0.25 | (L/kg) |
| VZ | 0.36 | 0.35 | 0.24 | 0.32 | (L/kg) |
| AUC0-t | 634.82 | 667.79 | 860.1 | 720.9 | (min*μmol/L) |
| AUC0-∞ | 673.47 | 704.18 | 878.17 | 751.94 | (min*μmol/L) |
| CLP | 3.46 | 3.48 | 2.83 | 3.26 | (mL/min/kg) |
| MRT | 68.66 | 61.13 | 48.84 | 59.54 | (min) |

TABLE 13
Plasma concentration (ng/mL) of D11.3 after IV (1.00 mg/kg) dose
administration in male Swiss Albino mice

| Time (h) | M1- | M2- | M3- | Mean | Std Dev | % CV |
|---|---|---|---|---|---|---|
| 0.08 | 3319.8 | 4091.53 | 3825.09 | 3745.47 | 391.98 | 10.47 |
| 0.25 | 3305.02 | 3779.15 | 2973.54 | 3352.57 | 404.9 | 12.08 |
| 0.5 | 1656.03 | 2566.56 | 1814.39 | 2012.33 | 486.47 | 24.17 |
| 1 | 931.71 | 1218.22 | 1283.47 | 1144.47 | 187.12 | 16.35 |
| 3 | 887.86 | 328.77 | 318.62 | 511.75 | 325.76 | 63.66 |
| 5 | 240.02 | 271.55 | 289.17 | 266.91 | 24.9 | 9.33 |
| 8 | 399.46 | 124.26 | 182.53 | 235.42 | 145.02 | 61.6 |
| 24 | 17.65 | 30.97 | 34.16 | 27.59 | 8.76 | 31.74 |
| Dose (mg/kg) | 1 | 1 | 1 | 1 | 0 | 0 |
| C0 (ng/mL) | — | — | — | 3957.56 | — | — |
| T½ (h) | — | — | — | 5.58 | — | — |
| AUC0-last | — | — | — | 6965.11 | — | — |
| (ng · h/mL) | | | | | | |
| AUC0-inf | — | — | — | 7187.15 | — | — |
| (ng · h/mL) | | | | | | |
| AUCExtra (%) | — | — | — | 3.09 | — | — |
| Cl | — | — | — | 2.32 | — | — |
| (ml/min/kg) | | | | | | |
| Vd (L/kg) | — | — | — | 1.12 | — | — |
| MRT0-last (h) | — | — | — | 4.64 | — | — |
| Rsq | — | — | — | 0.9904 | — | — |

TABLE 14
Plasma concentration (ng/mL) of D11.3 after PO (10.00 mg/kg) dose
administration in male Swiss Albino mice

| Time (h) | M1- | M2- | M3- | Mean | Std Dev | % CV |
|---|---|---|---|---|---|---|
| 0.08 | 47.49 | 76.49 | 84.94 | 69.64 | 19.64 | 28.21 |
| 0.25 | 454 | 804.64 | 992.29 | 750.31 | 273.23 | 36.42 |
| 0.5 | 1880.71 | 2858.02 | 2832.99 | 2523.91 | 557.17 | 22.08 |
| 1 | 2950.38 | 3417.67 | 2525.44 | 2964.5 | 446.28 | 15.05 |
| 3 | 3249.94 | 3660.57 | 3578.88 | 3496.46 | 217.37 | 6.22 |
| 5 | 3313.72 | 2427.94 | 1866.9 | 2536.19 | 729.46 | 28.76 |
| 8 | 1232.5 | 1764.28 | 1439.78 | 1478.85 | 268.03 | 18.12 |
| 24 | 177.35 | 48.47 | 59.29 | 95.04 | 71.49 | 75.22 |
| Dose (mg/kg) | 10 | 10 | 10 | 10 | 0 | 0 |
| Cmax | — | — | — | 3496.46 | — | — |
| (ng/mL) | | | | | | |
| Tmax (h) | — | — | — | 3 | — | — |
| AUC0-last | — | — | — | 28242.38 | — | — |
| (ng · h/mL) | | | | | | |
| AUC0-inf | — | — | — | 28793.58 | — | — |
| (ng · h/mL) | | | | | | |
| AUCExtra (%) | — | — | — | 1.91 | — | — |
| MRT0-last (h) | — | — | — | 6.32 | — | — |
| F (%) | — | — | — | 40.06 | — | — |
| Rsq | — | — | — | 1 | — | — |

TABLE 15
Plasma concentration (ng/mL) of D11.3 after SC (5.00 mg/kg) dose
administration in male Swiss Albino mice

| Time (h) | M1- | M2- | M3- | Mean | Std Dev | % CV |
|---|---|---|---|---|---|---|
| PD | 0 | 0 | 0 | 0 | 0 | #DIV/0! |
| 0.08 | 12.41 | 9.86 | 8.17 | 10.15 | 2.13 | 21.04 |
| 0.25 | 60.87 | 22.1 | 22.16 | 35.04 | 22.37 | 63.83 |
| 0.5 | 22.46 | 23.29 | 70.26 | 38.67 | 27.36 | 70.75 |
| 1 | 52.72 | 45.05 | 34.59 | 44.12 | 9.1 | 20.63 |
| 3 | 141.22 | 103.85 | 139.25 | 128.11 | 21.03 | 16.42 |
| 5 | 117.42 | 136.91 | 188.23 | 147.52 | 36.58 | 24.8 |
| 8 | 153.34 | 93.4 | 96.35 | 114.36 | 33.79 | 29.54 |
| 24 | 57.62 | 60.78 | 33.12 | 50.51 | 15.14 | 29.98 |
| Dose (mg/kg) | 5 | 5 | 5 | 5 | 0 | 0 |
| Cmax | — | — | — | 147.52 | — | — |
| (ng/mL) | | | | | | |
| Tmax (h) | — | — | — | 5 | — | — |
| AUC0-last | — | — | — | 2122.81 | — | — |
| (ng · h/mL) | | | | | | |
| AUC0-inf | — | — | — | 3047.15 | — | — |
| (ng · h/mL) | | | | | | |
| AUCExtra (%) | — | — | — | 30.33 | — | — |
| MRT0-last (h) | — | — | — | 10.71 | — | — |
| F (%) | — | — | — | 8.48 | — | — |
| Rsq | — | — | — | 0.9933 | — | — |

### Referenes

1. Nielsen, D. S. et al. Orally Absorbed Cyclic Peptides. Chem. Rev. 117, 8094-8128 (2017).
2. Modell, A. E., Blosser, S. L. & Arora, P. S. Systematic Targeting of Protein-Protein Interactions. Trends Pharmacol. Sci. 37, 702-713 (2016).
3. Räder, A. F. B. et al. Orally active peptides—is there a magic bullet? Angew. Chem. Int. Ed Engl. (2018) doi: 10.1002/anie.201807298.
4. Muttenthaler, M., King, G. F., Adams, D. J. & Alewood, P. F. Trends in peptide drug discovery. Nat. Rev. Drug Discov. (2021) doi: 10.1038/s41573-020-00135-8.
5. Hewitt, W. M. et al. Cell-permeable cyclic peptides from synthetic libraries inspired by natural products. J. Am. Chem. Soc. 137, 715-721 (2015).
6. Bockus, A. T. et al. Probing the Physicochemical Boundaries of Cell Permeability and Oral Bioavailability in Lipophilic Macrocycles Inspired by Natural Products. J. Med. Chem. 58, 4581-4589 (2015).
7. Bockus, A. T. & Lokey, R. S. Bioactive and Membrane-Permeable Cyclic Peptide Natural Products. Practical Medicinal Chemistry with (2017).
8. Peraro, L. & Kritzer, J. Getting in: emerging methods and design principles for cell-penetrant peptides. Angew. Chem. Int. Ed Engl. (2018) doi: 10.1002/ange.201801361.
9. Rezai, T. et al. Conformational flexibility, internal hydrogen bonding, and passive membrane permeability: successful in silico prediction of the relative permeabilities of cyclic peptides. J. Am. Chem. Soc. 128, 14073-14080 (2006).
10. Rand, A. C. et al. Optimizing PK properties of cyclic peptides: the effect of side chain substitutions on permeability and clearance( ). Medchemcomm 3, 1282-1289 (2012).
11. Wang, C. K., Swedberg, J. E., Harvey, P. J., Kaas, Q. & Craik, D. J. Conformational Flexibility Is a Determinant of Permeability for Cyclosporin. J. Phys. Chem. B 122, 2261-2276 (2018).
12. White, T. R. et al. On-resin N-methylation of cyclic peptides for discovery of orally bioavailable scaffolds. Nat. Chem. Biol. 7, 810-817 (2011).
13. Wang, C. K. et al. Rational design and synthesis of an orally bioavailable peptide guided by NMR amide temperature coefficients. Proc. Natl. Acad. Sci. U.S.A 111, 17504-17509 (2014).
14. Hill, T. A. et al. Cyclic Penta- and Hexaleucine Peptides without N-Methylation Are Orally Absorbed. ACS Med. Chem. Lett. 5, 1148-1151 (2014).
15. Mulligan, V. K. The emerging role of computational design in peptide macrocycle drug discovery. Expert Opin. Drug Discov. 15, 833-852 (2020).
16. Hosseinzadeh, P. et al. Comprehensive computational design of ordered peptide macrocycles. Science 358, 1461-1466 (2017).
17. Bhardwaj, G. et al. Accurate de novo design of hyperstable constrained peptides. Nature 538, 329-335 (2016).
18. Di, L., Kerns, E. H., Fan, K., McConnell, O. J. & Carter, G. T. High throughput artificial membrane permeability assay for blood-brain barrier. Eur. J. Med. Chem. 38, 223-232 (2003).
19. Pye, C. R. et al. Nonclassical Size Dependence of Permeation Defines Bounds for Passive Adsorption of Large Drug Molecules. J. Med. Chem. 60, 1665-1672 (2017).
20. Marelli, U. K. et al. cis-Peptide Bonds: A Key for Intestinal Permeability of Peptides? Chemistry 21, 15148-15152 (2015).
21. Ganguly, H. K., Majumder, B., Chattopadhyay, S., Chakrabarti, P. & Basu, G. Direct evidence for CH···π interaction mediated stabilization of Pro-cisPro bond in peptides with Pro-Pro-aromatic motifs. J. Am. Chem. Soc. 134, 4661-4669 (2012).
22. Grathwohl, C. & Wüthrich, K. Nmr studies of the rates of proline cis-trans isomerization in oligopeptides. Biopolymers 20, 2623-2633 (1981).
23. Ovadia, O. et al. The effect of multiple N-methylation on intestinal permeability of cyclic hexapeptides. Mol. Pharm. 8, 479-487 (2011).
24. Fouché, M. et al. Design and Development of a Cyclic Decapeptide Scaffold with Suitable Properties for Bioavailability and Oral Exposure. ChemMedChem 11, 1048-1059 (2016).

### Supplementary Methods

### Computational Design of Structured Membrane-Permeable Peptides

We modified the Rosetta™ generalized kinematic closure (GeneralizedKIC) based macrocycle design protocol described previously (Bhardwaj et al. 2016; Hosseinzadeh et al. 2017; Mulligan et al. 2021; Hosseinzadeh et al. 2021) to allow us to design 6 to 12 amino acid cyclic peptides capable of traversing the lipid membranes and cellular barriers. All steps for macrocycle design were implemented in Rosetta™Scripts (Fleishman et al. 2011).

Briefly, we chose a macrocycle size and started design calculations by constructing a linear polyglycine backbone of the selected amino acid length using the Rosetta™ PeptideStubMover (Bhardwaj et al. 2016). In this work, we performed separate design runs for each macrocycle size between 6 to 12 amino acids. Next, we declared a bond between the ‘C’ atom from the last residue and ‘N’ atom of the first residue in the polyglycine peptide and set up the distance, angle, and dihedral constraints for N-to-C terminal cyclization using the Rosetta™ PeptideCyclizeMover mover (Hosseinzadeh et al. 2017). We selected one residue randomly as the ‘anchor residue’ and three additional residues as the ‘pivot residues’. The omega torsions for all residues were set to 180°; § and v torsions for the anchor residues were randomly selected from a flat bottom mirror-symmetric Ramachandran table using the SetTorsion mover (Bhardwaj et al. 2016). Following, we used the GeneralizedKIC mover to identify cyclic polyglycine peptides from the linear peptides (Bhardwaj et al. 2016). Within the GenKIC mover, the $ and w dihedrals of the non-pivot and non-anchor residues were randomly drawn from a flat bottom mirror-symmetric Ramachandran table. The & and w dihedrals for the pivot residues were calculated analytically by the kinematic closure algorithm (Coutsias et al. 2004; Mandell, Coutsias, and Kortemme 2009; Bhardwaj et al. 2016) to find a combination of dihedral angles that give N-to-C cyclic peptide backbones. The criteria for closure were further defined to include a minimum number of internal backbone-to-backbone hydrogen bonds. The number of internal hydrogen bonds required was based on the length of the macrocycle: a minimum of 1 internal hydrogen bond was required for 6-7 amino acids, 2 hydrogen bonds were required for 8-9 amino acids, and 3 internal hydrogen bonds were required for macrocycles with 10 or more amino acids. In cases where GenKIC returned multiple cyclic solutions, we selected the lowest energy solutions based on a custom Rosetta™ energy function that includes only the fa_rep, fa atr, hbond sr_bb, hbond_Ir_bb, rama_prepro, and p_aa_pp score terms (Alford et al. 2017). For each successfully closed cyclic backbone, we used the Rosetta™ FastDesign mover to design an amino acid sequence that tried to minimize the overall energy of the macrocycle (Bhardwaj et al. 2016).

Since our goal was to design macrocycles that can traverse the lipid membranes passively, we removed NH groups not involved in hydrogen bonding by mutating the amino acids with such ‘unsatisfied’ backbone NH groups to their N-methyl variants. Residues with the unsatisfied NH groups were selected using the Unsat selector and mutated using the Modify VariantType mover to their N-methylated versions. However, given the different torsional preferences of the N-methylated amino acids, it is possible that the mutations to N-methyl amino acids could expose NH groups from other amino acids as well. Therefore, we followed an iterative approach with three rounds of amino acid sequence design and N-methylation of exposed backbone NH groups in-between. In the first round of designs we used an energy function with upweighted (5×) backbone hydrogen bonding score terms to favor more internal backbone-to-backbone hydrogen bonds. For the second round of design, we used the standard Rosetta™ beta_nov16 weights with constraints (Park et al. 2016; Alford et al. 2017). In the third round of design, we also allowed for cartesian minimization. We only allowed hydrophobic amino acids during design, and only D-amino acids were allowed at residue positions with positive φ, and only L-amino acids were allowed at residues with negative φ values. For some runs, we also used the AddCompositionConstraintMover to limit the minimum and maximum number of allowed prolines, D-prolines, and some bulky hydrophobic amino acids in the designed peptides (Hosseinzadeh et al. 2017). Given the difficulty in synthesizing peptides with multiple N-methylated amino acids, we filtered the design models based on total number N-methylated amino acids in the design models and lack of any exposed NH groups in the final designed state using Rosetta™ SimpleMetrics (Adolf-Bryfogle et al. 2021).

For each selected size range, approximately 10⁵ designs were sampled. Design models were clustered based on the torsion bin strings calculated from the backbone dihedral angles as described previously (Hosseinzadeh et al. 2017). Next, we pursued structure prediction of lowest-energy scoring designs from diverse clusters using Rosetta™ simple_cycpep_predict application (Bhardwaj et al. 2016; Hosseinzadeh et al. 2017) as described previously (Hosseinzadeh et al. 2017). We evaluated the Energy vs. RMSD-to-Design plots (Figure S2) from structure prediction calculations and selected the structured and conformation-switching peptides based on the number of low-energy states.

### Multistate Design of Conformation-Switching Peptides

We used a multistate design method to generate chameleonic macrocycles. Specifically, we implemented a genetic algorithm in PyRosetta™-3 that optimizes mutations to obtain two isoenergetic low energy states for the designed amino acid sequences. The starting sequence is used to generate a list of 1000 variants that contain mutations to different hydrophobic residues while maintaining the original chirality and N-methylation patterns. Each mutant sequence is then threaded onto the original backbone conformations and scored using the REF2015 Rosetta™ energy function (Park et al. 2016). The sequences are filtered to assure that the total energy of the sequence on both backbones is less than 10 kcal/mol and that the difference between the two states is less than 6 kcal/mol. In order to select for sequences that stabilize both conformations while maintaining low energy, sequences are then given a final score equal to (−5*abs(eA−eB))−(eA−eB) where eA and eB are the Rosetta™ scores of a given sequence threaded onto the first and second conformations, respectively. The best 500 sequences based on this metric are carried on to the next cycle of evaluation, where a point mutant of each sequence is added to the list, and the process is repeated. This algorithm was run for 1,000 generations and then the best sequence was chosen. Structure prediction is performed to ensure both desired conformational states are featured as low-energy minima in the conformational landscape.

### Conversion of Structured Peptides to Conformation-Switching Peptides

For some of the crystallographic confirmed designs, we attempted to identify amino acid substitutions that could create secondary isoenergetic minima. We implemented a PyRosetta™ script that loops through each amino acid position of a given structure and mutates the original residue to other hydrophobic amino acids while maintaining chirality and N-methylation patterns (see supplementary files for scripts and required files). Each mutated version of the original structure was then energy minimized using the Rosetta™ FastRelax protocol (Bhardwaj et al. 2016) to ease any strain induced by the mutation. The structure-energy landscape for the full set of mutated sequence-structure pairs was evaluated using Rosetta™ cycpep_predict application (Hosseinzadeh et al. 2017; Bhardwaj et al. 2016) and designs with isoenergetic alternate states separated by cis/trans isomerization were selected for experimental characterization.

### Structure Prediction of the Designed Macrocycles

We used the Rosetta™ simple_cycpep_predict application as described previously (Bhardwaj et al. 2016; Hosseinzadeh et al. 2017) to evaluate and conformational landscape for the designed amino acid sequences of macrocycles. We ran the structure prediction calculations using the Rosetta™ @Home platform. For each macrocycle, we generated >10⁴ energy minimized cyclic conformations of the designed amino acid sequence. For each conformation, we calculated the RMSD to the design model and the energy using the Rosetta™ REF2015 energy function (Park et al. 2016).

After the completion of the structure prediction runs, we plotted the Energy vs. RMSD to the design model and selected the design models that converged to the design model as the unique low-energy states or as one of the alternate isoenergetic states.

### Peptide Synthesis and Purification

Macrocyclic peptides were either purchased from Wuxi AppTec™ at greater than 99% purity or synthesized in-house using the Fmoc-based solid-phase peptide synthesis methods on an automated CEM Liberty Blue™ peptide synthesizer at a 0.1 mmol scale. Linear peptides were assembled on Cl-TCP (Cl) ProTide™ resin purchased from CEM, using standard coupling of 2 minutes at 90° C. and deprotection for 1 minute at 90° C., unless the Fmoc-amino acid was being coupled to an N-methylated amino acid in which case coupling was performed for 10 minutes at 90° C. We used Oxyma™ Pure/DIC as the coupling agent and 20% piperidine in DMF for deprotection as per standard protocols by CEM. Linear, protected peptides were released from the resin by repeated washes with 1% TEA in DCM. The wash volumes containing protected peptide were ejected into a round bottom flask containing a 50:50 mixture of acetonitrile and water of greater volume than the volume of the washes. DCM was removed by rotary evaporation and the resulting mixture was lyophilized to dryness. Dry, protected peptides were solubilized in DCM and placed on a stir plate with a stir bar. 2 equivalents of PyAOP were added directly to the solution followed by 5 equivalents of DIEA and left to stir overnight at room temperature. DCM was removed by rotary evaporation leaving an oil-like solution in the round bottom flask, which was resuspended in 50:50 acetonitrile and water and purified by reverse-phase high-performance liquid chromatography (RP-HPLC), Peptides were purified on an Agilent Infinity™ 1260 HPLC using an Agilent ZORBAX™ SB-C18, 80 Å, 5 μm, 9.4×250 mm column with a gradient of solvent A: 0.1% TFA in water, and solvent B: 0.1% TFA in acetonitrile. Mass spectrometry was used to confirm the synthesis of the correct product; purified peptides were direct-injected on a Thermo TSQ Quantum Access™ mass spectrometer.

### Parallel Artificial Membrane Permeability Assay (PAMPA)

Passive permeability was assayed using standard methods on a Corning® BioCoat™ Pre-coated PAMPA Plate System (Kansy, Senner, and Gubernator 1998). Starting stock solutions of peptides were prepared by adding 1-2 mg of peptide in 1 ml of DMSO solution, Stock solutions were diluted 20× in Phosphate buffered saline (PBS) buffer to create solutions with 5% DMSO. 300 μL of peptide solution was added to the donor well and 250 uL of 5% DMSO 1×PBS was added to the acceptor well. Donor and acceptor plates were incubated together for 16-20 hours and transferred to 96-well plates at the end of incubation for measuring concentrations of peptide in starting solution, donor wells, and acceptor wells using an RP-HPLC and mass spectrometry on Agilent 6230 LC/TOF. Samples were separated on a 20%/min gradient of Solvent A (water, 0.1% formic acid) and Solvent B (acetonitrile, 0.1% formic acid) ran using Acquity UPLC BEH C18 1.7 μm column. The area under the curve for the peaks matching the peptide mass was calculated, and peptide concentrations were calculated by fitting sample peak areas to a calibration curve of an 8 point two-fold serial dilution series from the starting donor solution. Propranolol and Cyclosporine were used as positive controls during the PAMPA. Apparent permeability (Pₐₚₚ) were calculated as follows:

$${P}_{app} = - ln ( 1 - {C}_{A} / {C}_{e} ) / A * ( 1 / {V}_{D} + 1 / {V}_{A} ) * t ; \\ {C}_{e} = ( {C}_{D} * {V}_{D} + {C}_{A} * {V}_{A} ) / ( {V}_{D} + {V}_{A} ) ;$$

t=incubation time(s),
C_{A}=compound concentration in the acceptor well at t,
C_{D}=compound concentration in the donor well at t,
Cₑ=compound at equilibrium concentration,
V_{A}=acceptor well volume,
V_{D}=donor well volume,
A=filter area.
   Single-Crystal x-Ray Diffraction of Peptides

Crystals diffraction data were collected from a single crystal at the synchrotron (on APS 24ID-C) and at 100 K. Unit cell refinement, and data reduction were performed using XDS and CCP4 suites (Kabsch 2010; Winn et al. 2001). The structure was identified by direct methods and refined by full-matrix least-squares on F² with anisotropic displacement parameters for the non-H atoms using SHELXL™-2016 (Sheldrick 2015b, [a] 2015). Structure analysis was aided by using Coot/Shelxle™ (Emsley and Cowtan 2004; Hübschle 2011). The hydrogen atoms on heavy atoms were calculated in ideal positions with isotropic displacement parameters set to 1.2×U_{eq} of the attached atoms. Since direct methods can return initial models with mirror handedness in 50% of the cases, we refined the structures with the handedness of the initial model from direct methods, and inverted using Rosetta™ flip_chirality mover for alignment and RMSD calculation. Structural coordinates of the refined X-ray structures deposited to the Cambridge Crystallography Data Centre (CCDC) with the following identifiers: 2131411 (D7.6), 2131412 (D7.8), 2131449 (D8.1), 2131417 (D8.2), 2131450 (D8.3.p1), 2131423 (D8.5.p2), 2131424 (D8.6), 2131249 (D8.9), 2131251 (D8.10), 2131252 (D8.12), 2131463 (D8.13), 2131425 (D8.14), 2131426 (D8.15), 2131427 (D8.17), 2131428 (D8.19), 2131253 (D8.25), 2131291 (D8.26), 2131429 (D9.1), 2131430 (D9.8), 2131245 (D9.24), 2131431 (D9.29), 2131432 (D9.30), 2131433 (D10.1), 2131434 (D10.21), 2131435 (D10.22), 2131436 (D10.23), 2131438 (D10.31 IPA), 2131437 (D10.31 ETP), 2131439 (D10.65), 2131440 (D11.1), 2131451 (D11.3), 2131441 (D11.4), 2131442 (D11.25), 2131292 (D8.31), 2131443 (D8.21), 2131293 (D9.16).

### In Vivo Oral Bioavailability Assays

### Evaluating Pharmacokinetic Properties of D8.3.p1 and D10.1

As a preliminary safety screen, peptides were first tested for cytotoxicity in vitro against two mammalian cell lines. CRL-8155 human lymphocyte and HepG2 human hepatocyte cells are seeded in 96-well plates and incubated for 48 hours in the presence of test peptide (serial-2 dilutions, from 80 μM to 1.25 μM, in triplicate). At the end of the incubation period, cells were visually assessed before alamarBlue™, a resazurin-based cell viability reagent which measures metabolic activity is added to the plate, and fluorescence was measured on a microplate reader. Fluorescence signals resulting from cell viability changes were compared with control wells to calculate 50% cytotoxic concentration (CC₅₀) values.

Pharmacokinetic properties were evaluated in Balb/C female mice (10-12 weeks old, avg. weight 20 g, in triplicates) after dosing the peptides through oral gavage (PO), subcutaneous (SQ), and intravenous (IV) routes. Blood samples were collected at multiple time points and plasma was separated from whole blood using centrifugation and stored at −80° C. Drug was extracted from plasma using 80% Acetonitrile (ACN) in water with 0.1% formic acid (FA) and an internal standard. Samples were mixed, centrifuged, and supernatant harvested for LC-MS analysis. For RP-HPLC analysis, peptides were evaluated using Agilent ZORBAX™ Eclipse Plus C18 1.8 μm, 2.1 mm×50 mm and Waters Xevo TQ-S micro Triple Quadrupole/ACQUITY™ UPLC H-Class. A two-component system composed of mobile phase A (0.1% FA in water) and mobile phase B (0.1% FA in 100% ACN) was used at a flow rate of 0.25 mL/min. Peptides were bound to an Agilent ZORBAX™ C18 column at 40° C. for 1 min with 95% mobile phase A, and then were eluted with a linear gradient from 5 to 95% mobile phase B for 4 min. MS was operated in multiple reaction monitoring (MRM) mode via the positive electrospray ionization interface using two transitions: m/z 886.6/128 and m/z 886.6/156 for D8.3.p1, and m/z 1139.92/99.97 for D10.1. Pharmacokinetic parameters were modeled using the Phoenix WinNonlin™ software package.

### Experimental Parameters for D8.3.p1

Dosing vehicle: 5% DMSO/5% Tween 80/90% PBS (PO, SQ); 5% DMSO/5% Tween 80/20% PEG 400/70% D5W (IV)
Dose volume: 200 mL (PO, SQ); 50 mL (IV)
Dose: 5 mg/kg, 0.5 mg/mL (PO, SQ); 1 mg/kg, 0.4 mg/mL (IV)
Sampling times: 15, 30, 60, 120, 240, 360, 1440 min (PO, SQ); 5, 15, 30, 60, 120, 240, 360 min (IV)

### Experimental Parameters for D10.1

Dosing vehicle: 5% DMSO/5% Tween 80/90% PBS (PO, SQ); 5% DMSO/5% Tween 80/20%
PEG 400/70% D5W (IV)
Dose volume: 200 mL (PO, SQ); 50 mL (IV)
Dose: 12.2 mg/kg, 1.22 mg/mL (PO, SQ); 2.45 mg/kg, 0.98 mg/mL (IV)
Sampling times: 15, 30, 60, 120, 240, 360, 1440 min (PO, SQ); 5, 15, 30, 60, 120, 240, 360 min (IV)

### Evaluating Pharmacokinetic Properties of D11.3

Pharmacokinetic properties of D11.3 were by GVK Bio in Swiss Albino male mice after dosing the peptides through oral gavage (PO), subcutaneous (SQ), and intravenous (IV) routes.

### Experimental Parameters for D11.3

Dosing vehicle: DMSO (10%)+10% solutol in PBS (90%) (IV, PO)
Dose volume: 200 mL (PO, SQ); 50 mL (IV)
Dose: 10 mg/kg (PO) 1 mg/kg (IV), 5 mg/kg (SQ)
Sampling times: 0.08, 0.25, 0.5, 1, 3, 5, 8, 24 h (IV, PO);

### REFERENCES

Adolf-Bryfogle, Jared, Jason W. Labonte, John C. Kraft, Maxim Shapovalov, Sebastian Raemisch, Thomas Lütteke, Frank DiMaio, et al. 2021. “Growing Glycans in Rosetta: Accurate de Novo Glycan Modeling, Density Fitting, and Rational Sequon Design.” bioRxiv. doi.org/10.1101/2021.09.27.462000.
Alford, Rebecca F., Andrew Leaver-Fay, Jeliazko R. Jeliazkov, Matthew J. O'Meara, Frank P. DiMaio, Hahnbeom Park, Maxim V. Shapovalov, et al. 2017. “The Rosetta All-Atom Energy Function for Macromolecular Modeling and Design.” Journal of Chemical Theory and Computation 13 (6): 3031-48.
Bhardwaj, Gaurav, Vikram Khipple Mulligan, Christopher D. Bahl, Jason M. Gilmore, Peta J. Harvey, Olivier Cheneval, Garry W. Buchko, et al. 2016. “Accurate de Novo Design of Hyperstable Constrained Peptides.” Nature 538 (7625): 329-35.
Coutsias, Evangelos A., Chaok Seok, Matthew P. Jacobson, and Ken A. Dill. 2004. “A Kinematic View of Loop Closure.” Journal of Computational Chemistry 25 (4): 510-28.
Emsley, Paul, and Kevin Cowtan. 2004. “Coot: Model-Building Tools for Molecular Graphics.” Acta Crystallographica. Section D, Biological Crystallography 60 (Pt 12 Pt 1): 2126-32.
Fleishman, Sarel J., Andrew Leaver-Fay, Jacob E. Corn, Eva-Maria Strauch, Sagar D. Khare, Nobuyasu Koga, Justin Ashworth, et al. 2011. “RosettaScripts: A Scripting Language Interface to the Rosetta Macromolecular Modeling Suite.” PloS One 6 (6): e20161.
Hosseinzadch, Parisa, Gaurav Bhardwaj, Vikram Khipple Mulligan, Matthew D. Shortridge, Timothy W. Craven, Fátima Pardo-Avila, Stephen A. Rettie, et al. 2017. “Comprehensive Computational Design of Ordered Peptide Macrocycles.” Science 358 (6369): 1461-66.
Hosseinzadeh, Parisa, Paris R. Watson, Timothy W. Craven, Xinting Li, Stephen Rettie, Fátima Pardo-Avila, Asim K. Bera, et al. 2021. “Anchor Extension: A Structure-Guided Approach to Design Cyclic Peptides Targeting Enzyme Active Sites.” Nature Communications 12 (1): 3384.
Hübschle, Shelxle. 2011. “CB; Sheldrick, GM; Dittrich, B.” Journal of Applied Crystallography 44:1281-84
Kabsch, Wolfgang. 2010. “XDS.” Acta Crystallographica. Section D, Biological Crystallography 66 (Pt 2): 125-32.
Kansy, M., F. Senner, and K. Gubernator. 1998. “Physicochemical High Throughput Screening: Parallel Artificial Membrane Permeation Assay in the Description of Passive Absorption Processes.” Journal of Medicinal Chemistry 41 (7): 1007-10.
Mandell, Daniel J., Evangelos A. Coutsias, and Tanja Kortemme. 2009. “Sub-Angstrom Accuracy in Protein Loop Reconstruction by Robotics-Inspired Conformational Sampling.” Nature Methods 6 (8): 551-52.
Mulligan, Vikram Khipple, Sean Workman, Tianjun Sun, Stephen Rettie, Xinting Li, Liam J. Worrall, Timothy W. Craven, et al. 2021. “Computationally Designed Peptide Macrocycle Inhibitors of New Delhi Metallo-β-Lactamase 1.” Proceedings of the National Academy of Sciences of the United States of America 118 (12): e2012800118.
Park, Hahnbeom, Philip Bradley, Per Greisen Jr, Yuan Liu, Vikram Khipple Mulligan, David E. Kim, David Baker, and Frank DiMaio. 2016, “Simultaneous Optimization of Biomolecular Energy Functions on Features from Small Molecules and Macromolecules.” Journal of Chemical Theory and Computation 12 (12): 6201-12.
Sheldrick, George M. 2015a. “Crystal Structure Refinement withSHELXL.” Acta Crystallographica Section C Structural Chemistry. doi.org/10.1107/s2053229614024218.
2015b. “SHELXT—Integrated Space-Group and Crystal-Structure Determination.” Acta Crystallographica Section A: Foundations and Advances 71 (1): 3-8.
Winn, M. D., Maximum-Likelihood Structure Refinemen, N. J. Pannu, G. N. Murshudov, and Others. 2001. “CCP4 v6. 3.0 Program References Any Publication Arising from Use of the CCP4 Software Suite Should Include Both References to the Specific Programs Used (see Below) and the Following Reference to the CCP4 Suite.” Acta Crystallographica 57:122-33.

We claim:

1. A cyclic peptide comprising or consisting of an amino acid sequence having no more than 3 amino acid substitutions compared to the amino acid sequence selected from the group consisting of SEQ ID NO: 1-123 and 125-131 or mirror images thereof, wherein residues in uppercase are L amino acids, and residues in lower case are D amino acids, wherein an asterisk identifies the amino acid residue following the asterisk as being an N-methylated amino acid.

| SEQ ID NO | |
|---|---|
| | 6-mers |
| 1 | iPfiPf |
| 2 | pfaPPl |
| 3 | p*faPPl |
| 4 | PPVfPf |
| 5 | PP*VfPf |
| 6 | *faP*vFP |
| 7 | vf*LpFP |
| 8 | *Vf*LpFP |
| 9 | iP*fiP*f |
| 10 | faPvFP |
| | 7-mers |
| 11 | P*lvLPpI |
| 12 | lp*LIP*lL |
| 13 | LP*llPpl |
| 14 | lp*ALpPL |
| 15 | VIpFIpa |
| 16 | Pplf*Lvf |
| 17 | VIp*FIpa |
| | 8-mers |
| 18 | lLplLaPL |
| 19 | lvP*lliP*l |
| 20 | FLPplVpl |
| 21 | LPpLv*a*al |
| 22 | P*lliP*liv |
| 23 | pp*AfA*L*ll |
| 24 | lVpPlVpP |
| 25 | Plf*FP*llp |
| 26 | Plf*FP*ll*G |
| 27 | plVpp*LvI |
| 28 | P*llFpp*aI |
| 29 | v*l*A*avPpI |
| 30 | PpA*L*lIvP |
| 31 | pp*LlpP*ll |
| 129 | pP*llpP*ll |
| 32 | PBliPBli |
| 33 | ia*A*lPv*lP |
| 34 | f*a*Lpllpv |
| 35 | Pa*l*f*Lvap |
| 36 | V*LpLV*LpL |
| 37 | APpVA*L*lV |
| 38 | lV*l*llVpP |
| 39 | lp*la*lplA |
| 40 | Fpp*LIPpl |
| 41 | MpPll*avP |
| 42 | ap*LLP*lv*a |
| 43 | ap*lvap*lv |
| 44 | fiPpivP*f |
| 47 | FpPL*l*I*IL |
| 56 | pP*lIpP*Il |
| | 9 mers |
| 45 | pPBLBVP*lI |
| 46 | Lip*LlP*fVl |
| 48 | Pv*avPlv*Lf |
| 49 | Pav*L*FIAPl |
| 50 | *LvL*L*fvpvp |
| 51 | PlLLP*a*aIp |
| 52 | pA*F*la*lv*VP |
| 53 | A*l*lPlpp*lf |
| 54 | P*aV*l*llL*aP |
| 55 | v*A*A*l*lfppP |
| 57 | Vf*a*L*lIpPa |
| 58 | piIF*FPp*LI |
| 59 | PlI*avpPlP |
| 60 | PpPaI*l*lfa |
| 61 | P*aaV*lllpL |
| 62 | APpVMPv*l*a |
| 63 | p*aV*l*apPLP |
| 64 | i*aiPAvpPl |
| 65 | piPP*lvI*la |
| 66 | lLp*LlP*aMa |
| 67 | vpPa*l*laVp |
| 68 | plAP*aLi*lV |
| 69 | lVI*I*FVPGP |
| 70 | pAllp*FLvP |
| 71 | pPGIlVP*aI |
| 72 | pMlP*lLF*lV |
| 73 | PpAP*laFif |
| 74 | pIl*lp*FvVP |
| 92 | vV*LPVL*Lpl |
| 94 | Pv*L*f*fp*LiA |
| 130 | pv*M*alpPLP |
| | 10-mers |
| 75 | Ip*zFap*Lv*lPv |
| 76 | PpAv*LA*lp*VV |
| 77 | lL*LLP*LA*AlP |
| 78 | *L*FLp*LiPpAv |
| 79 | aLVp*LVP*lV*I |
| 80 | P*lAP*lfLV*L*L |
| 81 | fA*l*A*LPpViP |
| 82 | Pi*A*LLPpA*Lf |
| 83 | l*ApppPv*L*lA |
| 84 | FpPvV*l*a*Vpa |
| 85 | P*lFiPLlp*av |
| 86 | F*alIPpVP*al |
| 87 | P*af*LlpPa*LV |
| 88 | fip*A*lLLpPl |
| 89 | l*aLfipaPpL |
| 90 | M*a*L*alpapPL |
| 91 | vlpvpPp*Lf*L |
| 92 | *G*FLp*LiPpAv |
| 93 | vafI*l*aPpPM |
| 94 | *L*fLp*LiPpAv |
| 95 | aP*fpFpPALL |
| 96 | aGpf*F*FfafP |
| 97 | PvpVVPvpFF |
| 98 | Llp*ApP*lA*lV |
| 99 | P*a*lAlPpi*li |
| 100 | p*lvIp*ApL*ll |
| 101 | apPvl*l*lAlP |
| 102 | PviP*a*Lplli |
| 103 | aPVP*faAV*fL |
| 104 | LVf*Ip*FlPLp |
| 105 | *L*GP*la*LP*ILF |
| 106 | *f*LPAV*I*FI*A*A |
| 107 | lILp*FVP*GF*L |
| 108 | LfPla*LL*AfP |
| | 11-mers |
| 109 | VPpL*iip*LIip |
| 110 | VPpL*iip*FIip |
| 111 | AP*lVliP*l*Ap*L |
| 112 | V*L*lAipPf*a*lP |
| 113 | V*l*LplapPAfl |
| 114 | pI*AipPlI*Av*L |
| 115 | Ppp*lf*L*lIAPv |
| 116 | PpVv*llpPI*AI |
| 118 | Lpp*LL*alp*la*l |
| 119 | Lf*L*app*lvPpa |
| 120 | P*aLPp*a*lF*lVI |
| 121 | PI*V*apPlV*lfp |
| 122 | vp*vl*IIP*liIP |
| 123 | ipLlp*LfL*aiP |
| 125 | lp*fapPpaall |
| 126 | ipLlp*LfL*giP |
| | 12-mers |
| 127 | p*FIpLlLpPa*aI |
| 128 | p*VIpLfLpPa*lI |
| 131 | VP*liPVAP*VlP*a |
| 117 | VP*liPVAP*vlP*a |

2. The cyclic peptide of claim 1, comprising or consisting of an amino acid sequence having no more than 3 amino acid substitutions compared to the amino acid sequence selected from the group consisting of SEQ ID NO: 1-123 and 125-128 or mirror images thereof.

3. The cyclic peptide of claim 1, comprising or consisting of an amino acid sequence having no more than 3 amino acid substitutions compared to the amino acid sequence selected from the group consisting of SEQ ID NO: SEQ ID NO:1-46, 48-55, 57-68, 70-91, 93, 96-103, 109-116, 118-123, 125, and 127-128, or mirror images thereof.

4. The cyclic peptide of any one of claims 1-3, wherein the cyclic peptide is cell membrane permeable.

5. The cyclic peptide of any one of claims 1-4, wherein the cyclic peptide comprises or consists of an amino acid sequence having no more than 3 amino acid substitutions compared to the amino acid sequence selected from the group consisting SEQ ID NO: 19, 36, 43, 61, 109, or mirror images thereof, wherein residues in uppercase are L amino acids, and residues in lower case are D amino acids, wherein an asterisk identifies the amino acid residue following the asterisk as being an N-methylated amino acid.

6. The cyclic peptide of any one of claims 1-5, wherein the cyclic peptide has at least one proline residue.

7. The cyclic peptide of any one of claims 1-5, wherein the cyclic peptide has at least two, three, four, or more proline residues.

8. The cyclic peptide of any one of claims 1-7, wherein the cyclic peptide has at least one N-methylated amino acid.

9. The cyclic peptide of any one of claims 1-7, wherein the cyclic peptide has at least two, three, or four N-methylated amino acids, or has two three, or four N-methylated amino acids.

10. The cyclic peptide of any one of claims 1-7, wherein the cyclic peptide has no N-methylated amino acids.

11. The cyclic peptide of any one of claims 1-10, wherein the cyclic peptide has at least two, three, four, five, six, or more proline and N-methylated amino acid residues in total.

12. The cyclic peptide of any one of claims 1-9 and 11, wherein N-methylated residues can only be substituted with (a) other N-methylated amino acids, (b) D-proline, or (d) L-proline.

13. The cyclic peptide of any one of claims 1-12, wherein D-proline residues can only be substituted with an N-methylated D-amino acid residue.

14. The cyclic peptide of any one of claims 1-3, wherein L-proline residues can only be substituted with an N-methylated L-amino acid residue.

15. The cyclic peptide of any one of claims 1-4, and wherein the peptide comprises at least 1, 2, 3, 4, or more D amino acids.

16. The cyclic peptide of any one of claims 1~15, wherein D-amino acid residues can only be substituted with other D-amino acid residues.

17. The cyclic peptide of any one of claims 1-6, wherein L-amino acid residues can only be substituted with other L-amino acid residues.

18. The cyclic peptide of any one of claims 1-17, wherein the cyclic peptide has at least 2, 3, 4, or more D amino acid residues.

19. The cyclic peptide of any one of claims 1-18, wherein the cyclic peptide has no more than 2 amino acid substitutions compared to the reference sequence.

20. The cyclic peptide of any one of claims 1-19, wherein the cyclic peptide has no more than 1 amino acid substitutions compared to the reference sequence.

21. The cyclic peptide of any one of claims 1-20, comprising or consisting the amino acid sequence of an amino acid sequence of the reference sequence, or a mirror image thereof.

22. The cyclic peptide of any one of claims 1-21, wherein the cyclic peptide is 6-12 amino acid residues in length.

23. A cyclic peptide comprising or consisting of a bin string as listed in Table 3, wherein, wherein:
A is a right-handed helical region;
B is a right-handed strand region;
X is a mirror image of A;
Y is a mirror image of B;
O are amino acids with phi <0 and cis peptide bond between the residue i and i+1; and
Z are amino acids with phi >0 and cis peptide bond between the residue i and i+1.

24. The cyclic peptide of claim 23, comprising the cyclic peptide of any one of claims 1-22.

25. The cyclic peptide of any one of claims 1-24, wherein the cyclic peptide has an apparent permeability greater than 1×10⁻⁷ cm/s.

26. The cyclic peptide of any one of claims 1-24, wherein the cyclic peptide has an apparent permeability greater than 1×10⁶ cm/s.

27. The cyclic peptide of claim 25 or 26, wherein the apparent permeability is measured by a rate of traversal across artificial membranes in parallel artificial membrane permeability assays (PAMPA), determined by mass spectrometry-based quantification of peptide concentrations in the donor and acceptor wells.

28. A conjugate, comprising the cyclic peptide of any one of claims 1-27 bound to a moiety.

29. The conjugate of claim 28, wherein the moiety comprises a therapeutic agent, a diagnostic agent, a marker, a linker, a dye, a purification tag, a peptide, a small molecule, or a nucleic acid.

30. A cyclic peptide library, comprising two or more cyclic peptides and/or conjugates according to any one of claims 1-29.

31. The cyclic peptide library of claim 30, comprising ten or more cyclic peptides and/or conjugates according to any one of claims 1-29.

32. The cyclic peptide library of claim 30, comprising fifty or more cyclic peptides and/or conjugates according to any one of claims 1-29.

33. The cyclic peptide library of claim 30, comprising two hundred or more cyclic peptides and/or conjugates according to any one of claims 1-29.

32. Use of the cyclic peptide, conjugate, or cyclic peptide library of any preceding claim to, for example, to carry a linked moiety across a cell membrane, or as a scaffold for target-based drug design, or to screen molecules of interest for binding to one or more of the cyclic peptides.
